(19) United States
(12) Patent Application Publication
(10) Pub. No.: US 2004/0234973 A1
(43) Pub. Date: Nov. 25, 2004

(54) Methods and nucleic acids for the analysis of hematopoietic cell proliferative disorders

(76) Inventors: Peter Adorjan, Berlin (DE); Ralf Lesche, Berlin (DE); Evelyne Lipscher, Berlin (DE); Sabine Maier, Berlin (DE); Fabian Model, Berlin (DE); Inko Nimmrich, Berlin (DE)

Correspondence Address: Kriegsman & Kriegsman, 665 Franklin Street, Framingham, MA, 01702, US

(21) Appl. No.: 10/473,126

(22) PCT Filed: Mar. 26, 2002
(86) PCT No.: PCT/EP02/03401

Related U.S. Application Data
(60) Provisional application No. 60/278,333, filed on Mar. 26, 2001.

(51) Int. Cl. C12Q 1/68; C12N 5/08

(52) U.S. Cl. 435/6; 435/372

(57) ABSTRACT

The present invention relates to modified and genomic sequences, to oligonucleotides and/or PNA-oligomers for detecting the cytosine methylation state of genomic DNA, as well as to a method for ascertaining genetic and/or epigenetic parameters of genes for use in the differentiation, diagnosis, treatment and/or monitoring of hematopoietic cell proliferative disorders, or the predisposition to hematopoietic cell proliferative disorders.

### FIELD OF THE INVENTION

Class prediction is of crucial importance for most therapeutic decisions in cancer, therefore prediction of tumour class is one of the most important problems in diagnostic oncology. Different classes and sub-classes of cancers respond differently to specific types of treatment. Vital therapeutic decisions such as which therapeutic regimen is used are therefore crucially dependent on the precise recognition of tumour class. Currently tumour classification generally depends on morphological, histopathological or immunological parameters, and single molecular markers (R. W. McKenna, Clin Chem. 46, 1252 (2000); F. R. Appelbaum, Semin Hematol. 36, 401 (1999)). However, none of the diagnostic procedures currently in use is sufficient to classify tumours correctly. Different methods have to be combined, and correct classification of a tumour depends on the experience of individual pathologists.

Recently, several groups have shown that precise determination of tumour class can be achieved by microarray-based expression analysis. Golub and coworkers screened the expression levels of almost 7000 genes, between 10 and 100 of which were then shown to be sufficient to distinguish between acute lymphoblastic leukaemia (ALL) and acute myeloid leukaemia (AML) (T. R. Golub et al., Science 286, 531 (1999)). In a similar approach, Alizadeh and coworkers discovered yet unknown subclasses of diffuse large B-cell lymphoma with significant differences regarding response to therapy and disease outcome (A. A. Alizadeh et al., Nature 403, 503 (2000)). Also, it was shown that global transcript analysis can predict phenotypic characteristics of malignant melanoma (M. Bittner et al., Nature 406, 535 (2000)).

Large scale analysis using mRNA based microarrays are primarily impeded by the instability of mRNA (T. Emmert-Buck et al., Am J Pathol. 156, 1109 (2000)). Also expression changes of only a minimum of a factor 2 can be routinely and reliably detected (R. J. Lipshutz, S. P. A Fodor, T. R. Gingeras, D. J. Lockhart, Nature Genetics 21, 20 (1999); D. W. Selinger, K. J. Cheung, R. Mei, E. M. Johansson, C. S. Richmond, Nature Biotechnology 18, 1262 (2000)). Furthermore, sample preparation is complicated by the fact that expression changes occur within minutes following certain triggers. The inability to resolve the individual contributions of such influences on an expression profile, and difficulties with quantifying the gradual nature of the occurring changes complicates data analysis.

Aberrant DNA methylation within CpG islands is common in human malignancies leading to abrogation or overexpression of a broad spectrum of genes (P. A. Jones, Cancer Res 65, 2463 (1996)). Abnormal methylation has also been shown to occur in CpG rich regulatory elements in intronic and coding parts of genes for certain tumours (M. F. Chan, G. Liang, P. A. Jones, Curr Top Microbiol Immunol 249, 75(2000)). Using restriction landmark genomic scanning, Costello and coworkers were able to show that methylation patterns are tumour-type specific (J. F. Costello et al., Nat Genet 24, 132 (2000)). Highly characteristic DNA methylation patterns could also be shown for breast cancer cell lines (T. H.-M. Huang, M. R. Perry, D. E. Laux, Hum Mol Genet 8, 459 (1999)). As does large scale mRNA expression monitoring, genome wide assessment of methylation status represents a molecular fingerprint of cancer tissues and therefore should allow tumour class prediction and discovery.

Leukemia is a malignant disease that originates in a cell in the marrow, and is characterized by the uncontrolled proliferation of developing marrow cells. The majority of leukemias are classified according to the cell type from which they develop, as either myelogenous or lymphocytic. Both classes may be acute or chronic. Acute leukemia is a rapidly progressing disease that results in the accumulation of immature, functionless cells in the marrow and blood. In many cases the marrow can no longer produce sufficient quantities of normal red and white blood cells and platelets. Anemia, a deficiency of red cells, develops in virtually all leukemia patients. The lack of normal white cells impairs the body's ability to fight infections. A shortage of platelets results in bruising and easy bleeding. Chronic leukemia progresses more slowly and permits greater numbers of more mature, functional cells to be made.

Among an estimated 31,500 new cases of leukemia in the United States in 2001, about equal proportions are acute leukemia and chronic types. Most cases occur in older adults; more than half of all cases occur after age 60. Leukemia usually strikes ten times as many adults as children. Leukemia is the most common childhood cancer and acute lymphocytic leukemia (hereinafter also referred to by the abbreviation ALL) accounts for 80 percent of the childhood leukemia cases. The most common types of leukemia in adults are acute myelogenous leukemia hereinafter also referred to by the abbreviation AML), with an estimated 10,000 new cases annually. Acute lymphocytic leukemia will account for about 3,500 cases this year.

In additon to environmental and viral causes a variety of genes involved in cellular pathways such as cell signalling, apoptosis, cell proliferation, and senescence are thought to be involved in the development of leukemia Examples of such genes in Bc12, ALL1,

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec. 15;24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfier W, Horsthemke B. A single-tube PCR test for the diagnosis of Angelman and Prader-Willi syndrome based on allelic methylation differences at the SNRPN locus. Eur J Hum Genet. 1997 March-April;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15;25(12):2529-31, WO Patent 9500669) or by enzymatic digestion (Xiong Z, Laird P W. COBRA: a sensitive and quantitative DNA methylation assay. Nucleic Acids Res. 1997 Jun. 15;25(12):2532-4). In addition, detection by hybridization has also been described (Olek et al., WO 99 28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Charlton J, Bird A P, Walter J, Reik W. Methylation analysis on individual chromosomes: improved protocol for bisulphite genomic sequencing. Nucleic Acids Res. 1994 Feb. 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):2614; WO 97 46705, WO 95 15373 and WO 45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionization of proteins with molecular masses exceeding 10,000 daltons. Anal Chem. 1988 Oct. 15;60(20):2299-301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallization. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

The method according to the invention presents a novel microarray-based assay which is suited for methylation analysis of very large numbers of genes and CpG dinucleotides in parallel. Samples obtained from patients with acute lymphoblastic leukemia (ALL) or acute myeloid leukemia (AML) can be classified based solely on DNA methylation patterns.

### DESCRIPTION

The invention provide a method for the analysis of biological samples for features associated with the development of hematopoietic cell proliferative disorders, characterised in that the nucleic acid of at least one member of the group comprising the genes: ABL1, ABL1, APAF1, APC, AR, ARHI, BAK1, BAX, BCL2, CASP10, CASP8, CASP9, CCND2, CDC2, CDC25A, CDH1, CDH3, CDK 4, CDKN1A, CDKN1B (p27 Kip1), CDKN1C, CDKN2a, CDKN2B, CSNK2B, DAPK1, EGR4, ELK1, ESR1, FOS, GPIb beta, GPR37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3 is/are contacted with a reagent or series of reagents capable of distinguishing between methylated and non methylated CpG dinucleotides within the genomic sequence of interest.

The present invention makes available a method for ascertaining genetic and/or epigenetic parameters of genomic DNA. The method is for use in the improved diagnosis, treatment and monitoring of hematopoietic cell proliferative disorders, more specifically by enabling the improved identification of and differentiation between subclasses of said disorder and the genetic predisposition to said disorders. The invention presents improvements over the state of the art in that it enables a highly specific classification of hematopoietic cell proliferative disorders, thereby allowing for improved and informed treatment of patients.

In a particularly preferred embodiment the present invention makes available methods and nucleic acids that allow the differentiation between acute lymphocytic leukemia and acute myelogenous leukemia.

Furthermore, the method enables the analysis of cytosine methylations and single nucleotide polymorphisms.

In a preferred embodiment, the method comprises the following steps:

In the first step of the method the genomic DNA sample must be isolated from sources such as cell lines or blood samples. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the next step of the method, this may be by any means standard in the state of the art, in particular, but not limited to, with restriction endonucleases.

In the second step of the method, the genomic DNA sample is treated in such a manner that cytosine bases which are unmethylated at the 5′-position are converted to uracil, thymine, or another base which is dissimilar to cytosine in terms of hybridization behaviour. This will be understood as ‘pretreatment’ hereinafter.

The above described treatment of genomic DNA is preferably carried out with bisulfite (sulfite, disulfite) and subsequent alkaline hydrolysis which results in a conversion of non-methylated cytosine nucleobases to uracil or to another base which is dissimilar to cytosine in terms of base pairing behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines.

Fragments of the pretreated DNA are amplified, using sets of primer oligonucleotides according to SEQ ID NO:387 to SEQ ID NO: 534, and a, preferably heat-stable, polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

The method may also be enabled by the use of alternative primers, the design of such primers is obvious to one skilled in the art. These should include at least two oligonucleotides whose sequences are each reverse complementary or identical to an at least 18 base-pair long segment of the base sequences specified in the appendix (SEQ ID NO:95 through SEQ ID NO: 386). Said primer oligonucleotides are preferably characterized in that they do not contain any CpG dinucleotides. In a particularly preferred embodiment of the method, the sequence of said primer oligonucleotides are designed so as to selectively anneal to and amplify, only the hematopoietic cell specific DNA of interest, thereby minimizing the amplification of background or non relevant DNA. In the context of the present invention, background DNA is taken to mean genomic DNA which does not have a relevant tissue specific methylation pattern, in this case, the relevant tissue being hematopoietic cells, both healthy and diseased.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminum, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionization mass spectrometry (MALDI) or using electron spray mass spectrometry (ESI).

The amplificates obtained in the second step of the method are subsequently hybridized to an array or a set of oligonucleotides and/or PNA probes. In this context, the hybridization takes place in the manner described as follows. The set of probes used during the hybridization is preferably composed of at least 10 oligonucleotides or PNA-oligomers. In the process, the amplificates serve as probes which hybridize to oligonucleotides previously bonded to a solid phase. In a particularly preferred embodiment, the oligonucleotides are taken from the group comprising SEQ ID NO: 535 to SEQ ID NO: 1258. In a further preferred embodiment as described in the examples, the oligonucleotides are taken from the group comprising SEQ ID NO: 1211 to SEQ ID NO: 1258. The non-hybridized fragments are subsequently removed. Said oligonucleotides contain at least one base sequence having a length of 10 nucleotides which is reverse complementary or identical to a segment of the base sequences specified in the appendix, the segment containing at least one CpG or TpG dinucleotide. In a further preferred embodiment the cytosine of the CpG dinucleotide, or in the case of TpG, the thiamine, is the 5^{th} to 9^{th} nucleotide from the 5′-end of the 10-mer. One oligonucleotide exists for each CpG or TpG dinucleotide.

In the fifth step of the method, the non-hybridized amplificates are removed.

In the final step of the method, the hybridized amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/ionization mass spectrometry (MALDI) or using electron spray mass spectrometry (ESI). The produced fragments may have a single positive or negative net charge for better detectability in the mass spectrometer.

The aforementioned method is preferably used for ascertaining genetic and/or epigenetic parameters of genomic DNA.

In order to enable this method, the invention further provides the modified DNA of genes ABL1, ABL1, APAF1, APC, AR, ARHI, BAK1, BAX, BCL2, CASP10, CASP8, CASP9, CCND2, CDC2, CDC25A, CDH1, CDH3, CDK 4, CDKN1A, CDKN1B (p27 Kip1), CDKN1C, CDKN2a, CDKN2B, CSNK2B, DAPK1, EGR4, ELK1, ESR1, FOS, GPIb beta, GPR37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3 as well as oligonucleotides and/or PNA-oligomers for detecting cytosine methylations within said genes. The present invention is based on the discovery that genetic and epigenetic parameters and, in particular, the cytosine methylation patterns of genomic DNA are particularly suitable for improved diagnosis, treatment and monitoring of hematopoietic cell proliferative disorders. Furthermore, the invention enables the differentiation between different subclasses of hematopoietic cell proliferative disorders or detection of a predisposition to hematopoietic cell proliferative disorders.

The nucleic acids according to the present invention can be used for the analysis of genetic and/or epigenetic parameters of genomic DNA.

This objective is achieved according to the present invention using a nucleic acid containing a sequence of at least 18 bases in length of the pretreated genomic DNA according to one of SEQ ID NO: 95 through SEQ ID NO: 386 and sequences complementary thereto.

The modified nucleic acid could heretofore not be connected with the ascertainment of disease relevant genetic and epigenetic parameters.

The object of the present invention is further achieved by an oligonucleotide or oligomer for the analysis of pretreated DNA, for detecting the genomic cytosine methylation state, said oligonucleotide containing at least one base sequence having a length of at least 10 nucleotides which hybridizes to a pretreated genomic DNA according to SEQ ID NO: 95 through SEQ ID NO: 386. The oligomer probes according to the present invention constitute important and effective tools which, for the first time, make it possible to ascertain specific genetic and epigenetic parameters during the analysis of biological samples for features associated with the development of hematopoietic cell proliferative disorders. Said oligonucleotides allow the improved diagnosis, treatment and monitoring of hematopoietic cell proliferative disorders and detection of the predisposition to said disorders. Furthermore, they allow the differentiation of different subclasses of hematopoietic carcinomas. The base sequence of the oligomers preferably contains at least one CpG or TpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides within SEQ ID NO: 95 through SEQ ID NO: 386. Preferred is a set which contains at least one oligomer for each of the CpG dinucleotides, from SEQ ID NO: 535 through SEQ ID NO: 1258. Further preferred is a set comprising SEQ ID NO: 1211 to SEQ ID NO: 1258.

In the case of the sets of oligonucleotides according to the present invention, it is preferred that at least one oligonucleotide is bound to a solid phase. It is further preferred that all the oligonucleotides of one set are bound to a solid phase.

The present invention moreover relates to a set of at least 10 n (oligonucleotides and/or PNA-oligomers) used for detecting the cytosine methylation state of genomic DNA using treated versions of said genomic DNA (according to SEQ ID NO: 95 through SEQ ID NO: 386 and sequences complementary thereto). These probes enable improved diagnosis, treatment and monitoring of hematopoietic cell proliferative disorders. In particular they enable the differentiation between different sub classes of hematopoietic cell proliferative disorders and the detection of a predisposition to said disorders. In a particularly preferred embodiment the set comprises SEQ ID NO: 74 to SEQ ID NO: 1258.

The set of oligomers may also be used for detecting single nucleotide polymorphisms (SNPs) using pretreated genomic DNA according to one of SEQ ID NO: 95 through SEQ ID NO: 386.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterized in that it is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are suitable alternatives.

Therefore, a further subject matter of the present invention is a method for manufacturing an array fixed to a carrier material for the improved diagnosis, treatment and monitoring of hematopoietic cell proliferative disorders, the differentiation between different subclasses of hematopoietic carcinomas and/or detection of the predisposition to hematopoietic cell proliferative disorders. In said method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the improved diagnosis, treatment and monitoring of hematopoietic cell proliferative disorders. Furthermore the DNA chip enables detection of the predisposition to hematopoietic cell proliferative disorders and the differentiation between different subclasses of hematopoietic carcinomas. The DNA chip contains at least one nucleic acid according to the present invention. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond to or are complementary to a 18 base long segment of the base sequences specified in the appendix (SEQ ID NO: 95 through SEQ ID NO: 386), oligonucleotides and/or PNA-oligomers as well as instructions for carrying out and evaluating the described method. However, a kit along the lines of the present invention can also contain only part of the aforementioned components.

The oligomers according to the present invention or arrays thereof as well as a kit according to the present invention are intended to be used for the improved diagnosis, treatment and monitoring of hematopoietic cell proliferative disorders. Furthermore the use of said inventions extends to the differentiation between different subclasses of hematopoietic carcinomas and detection of the predisposition to hematopoietic cell proliferative disorders. According to the present invention, the method is preferably used for the analysis of important genetic and/or epigenetic parameters within genomic DNA, in particular for use in improved diagnosis, treatment and monitoring of hematopoietic cell proliferative disorders, detection of the predisposition to said disorders and the differentiation between subclasses of said disorders.

The methods according to the present invention are used, for example, for improved diagnosis, treatment and monitoring of hematopoietic cell proliferative disorders progression, detection of the predisposition to said disorders and the differentiation between subclasses of said disorders.

A further embodiment of the invention is a method for the analysis of the methylation status of genomic DNA without the need for pretreatment. In the first step of the method the genomic DNA sample must be isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the treatment, this may be any means standard in the state of the art, in particular with restriction endonucleases. In the second step, the DNA is then digested with one or more methylation sensitive restriction enzymes. The digestion is carried out such that hydrolysis of the DNA at the restriction site is informative of the methylation status of a specific CpG dinucleotide.

In the third step the restriction fragments are amplified. In a preferred embodiment this is carried out using a polymerase chain reaction.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

The analysis of the methylation status of CpG positions using either of the molecular biological techniques described above in multiple patient samples generates a large set of data points which are then analysed using the following techniques in order to define CpG marker positions fpr a particular phenotypic class, in particular by using supervised machine learning techniques.

The problem of class prediction was addressed by using a supervised machine learning method called support vector machine (SVM) (V. Vapnik, Statistical Learning Theory (Wiley, New York 1998); N. Christianini, J. Shawe-Taylor, An Introduction to Support Vector Machines (Cambridge University Press, Cambridge 2000)), which has already been successfully applied to the analysis of microarray gene expression data (M. P. Brown et al., Proc Natl Acad Sci USA 97, 262(2000); T. Gaasterland, S. Bekiranov, Nature Genetics 24, 204(2000)). The SVM constructs an optimal discriminant between two classes of given training samples. In this case each sample is described by the methylation patterns (CG/TG ratios) at the investigated CpG sites. A supervised learning technique, such as SVM, has the advantage that it exploits the prior knowledge represented by data labels. Furthermore, SVMs are capable to account for highly non-linear interdependencies between individual dimensions (in this case CpG sites) and still avoid overfitting the data (V. Vapnik, Statistical Learning Theory (Wiley, New York 1998)).

In order to quantify the contribution of single CpG sites to the prediction of classes, we initially ranked all CpG sites according to their discriminatory power for the investigated sample classes using a two sample t-test. Then the SVM was trained using an increasing number of CpG sites in the order of their ranking. The number of CpG sites where the prediction error is minimized depends on which particular classes are to be separated from each other. This is explained by the fact that, on the one hand, the exclusion of certain CpG positions from the analysis potentially leads to a loss of information. On the other hand, as suggested by statistical learning theory (V Vapnik, Statistical Learning Theory (Wiley, New York 1998)), for a limited number of samples the reliability of the class-predictor generally increases with decreasing number of free parameters, in our case number of CpG sites. The inherent, task-specific complexity of the classification problem determines where these two counteracting effects are balanced and class prediction achieves its optimal performance., This suggests, that a proper feature selection in genome-wide analyses not only improves class prediction J. Weston et al., in Advances in neural information processing systems (MIT Press, Cambridge, Mass. 2001), but also provides information about the complexity of the problem.

The present invention moreover relates to the diagnosis and/or prognosis of events which are disadvantageous or relevant to patients or individuals in which important genetic and/or epigenetic parameters within genomic DNA, said parameters obtained by means of the present invention may be compared to another set of genetic and/or epigenetic parameters, the differences serving as the basis for the diagnosis and/or prognosis of events which are disadvantageous or relevant to patients or individuals.

In the context of the present invention the term “hybridization” is to be understood as a bond of an oligonucleotide to a completely complementary sequence along the lines of the Watson-Crick base pairings in the sample DNA, forming a duplex structure.

In the context of the present invention, “genetic parameters” are mutations and polymorphisms of genomic DNA and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further modifications of DNA bases of genomic DNA and sequences further required for their regulation. Further epigenetic parameters include, for example, the acetylation of histones which, cannot be directly analyzed using the described method but which, in turn, correlates with the DNA methylation.

In the following, the present invention will be explained in greater detail on the basis of the sequences and examples without being limited thereto.

SEQ ID NO: 1 to SEQ ID NO: 73 represent 5′ and/or regulatory regions of the genomic DNA of genes: ABL1, ABL1, APAF1, APC, AR, ARHI, BAK1, BAX, BCL2, CASP10, CASP8, CASP9, CCND2, CDC2, CDC25A, CDH1, CDH3, CDK 4, CDKN1A, CDKN1B (p27 Kip1), CDKN1C, CDKN2a, CDKN2B, CSNK2B, DAPK1, EGR4, ELK1, ESR1, FOS, GPIb beta, GPR37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3. These sequences are derived from Genbank and will be taken to include all minor variations of the sequence material which are currently unforeseen, for example, but not limited to, minor deletions and SNPs.

SEQ ID NO: 95 to SEQ ID NO: 386 exhibit the pretreated sequence of DNA derived from genes: ABL1, ABL1, APAF1, APC, AR, ARHI, BAK1, BAX, BCL2, CASP10, CASP8, CASP9, CCND2, CDC2, CDC25A, CDH1, CDH3, CDK 4, CDKN1A, CDKN1B (p27 Kip1), CDKN1C, CDKN2a, CDKN2B, CSNK2B, DAPK1, EGR4, ELK1, ESR1, FOS, GPIb beta, GPR37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3. These sequences will be taken to include all minor variations of the sequence material which are currently unforeseen, for example, but not limited to, minor deletions and SNPs.

SEQ ID NO: 387 to SEQ ID NO: 534 exhibit the sequence of primer oligonucleotides for the amplification of pretreated DNA according to SEQ ID NO: 95 to SEQ ID NO: 386.

SEQ ID NO: [&IDOLIGOFIRST] to SEQ ID NO: 1258 exhibit the sequence of oligomers which are useful for the analysis of CpG positions within genomic DNA according to SEQ ID NO: 1 to SEQ ID NO: 73.

SEQ ID NO: 1211 to SEQ ID NO: 1258 exhibit the sequence of oligomers which are useful for the analysis of CpG positions within genomic DNA according to SEQ ID NO: 1 to SEQ ID NO: 73.

### EXAMPLES 1 AND 2

### Digital Phenotype

In the following examples, multiplex PCR was carried out on samples from patients with acute lymphocytic leukemia and acute myelogenous leukemia. Each sample was treated in the manner described below in Example 1 in order to deduce the methylation status of CpG positions, the CpG methylation information for each sample was collated and then used in an analysis, as detailed in Example 2. An alternative method for the analysis of CpG methylation status is further described in Example 3.

### EXAMPLE 1

A total of 17 ALL samples and 8 AML samples were included. T- and B-cells from 8 healthy donors, MACS-sorted with anti-CD4 and anti-CD19 antibodies, respectively, served as controls. 11 genes were randomly selected from a panel of genes representing different pathways associated with tumourigenesis. Two of the 11 selected genes are located on the X-chromosome. A total of 81 CpG dinucleotides located in CpG rich regions of the promoters, intronic and coding sequences of these genes were evaluated for methylation status.

In order to allow sequence specific distinction of the methylated from the unmethylated state of CpG dinucleotides by hybridisation analysis, total DNA from all samples was bisulphite treated converting all unmethylated cytosines to uracil whereas methylated cytosines were conserved (M. Frommer et al., Proc Natl Acad Sci USA 89, 1827 (1992)). Regions of interest were then amplified by PCR using fluorescently labelled primers converting originally unmethylated CpG dinucleotides to TG and conserving originally methylated CpG sites. Primers were designed complementary to DNA segments containing no CpG dinucleotides. This allowed unbiased amplification of both methylated and unmethylated alleles in one reaction. All PCR products performed on an individual sample were mixed and hybridised to glass slides carrying for each CpG position a pair of immobilised oligonucleotides. Each of these detection oligonucleotides was designed to hybridise to the bisulphite converted sequence around one CpG site which was either originally unmethylated (TG) or methylated (CG). Hybridisation conditions were selected to allow the detection of the single nucleotide differences between the TG and CG variants. Ratios for the two signals were calculated based on comparison of intensity of the fluorescent signals. Sensitivity for detection of methylation changes was determined using artificially up- and downmethylated DNA fragments mixed at different ratios. For each of those mixtures, a series of experiments was conducted to define the range of CG/TG ratios that corresponds to varying degrees of methylation at each of the CpG sites tested.

In the first step the genomic DNA was isolated from the cell samples using the Wizzard kit from (Promega).

The isolated genomic DNA from the samples are treated using a bisulfite solution (hydrogen sulfite, disulfite). The treatment is such that all non methylated cytosines within the sample are converted to thiamidine, conversely 5-methylated cytosines within the sample remain unmodified. Bisulphite treatment of genomic DNA was done with minor modifications as described by A. Olek, J. Oswald, J. Walter, Nucleic Acid Res. 24, 5064 (1996). Genomic DNA was digested with MssI (MBI Fermentas, St. Leon-Rot, Germany) prior to the modification by bisulphite.

The treated nucleic acids were then amplified using multiplex PCRs, amplifying 8 fragments per reaction with Cy5 fluorescently labelled primers. PCR primers used are described in Table 1. PCR conditions were as follows.

For the PCR amplification of the bisulphite treated sense strand of the genes used for class prediction and discovery the primers were designed according to the guidelines of Clark and Frommer (S. J. Clark, M. Frommer, in Laboratory Methods for the Detection of Mutations and Polymorphisms in DNA, G. R. Taylor ed. (CRC Press, Boca Raton 1997)). CpG sites from the following genes were analysed: ABL1, ABL1, APAF1, APC, AR, ARHI, BAK1, BAX, BCL2, CASP10, CASP8, CASP9, CCND2, CDC2, CDC25A, CDH1, CDH3, CDK 4, CDKN1A, CDKN1B (p27 Kip1), CDKN1C, CDKN2a, CDKN2B, CSNK2B, DAPK1, EGR4, ELK1, ESR1, FOS, GPIb beta, GPR37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3.

10 ng DNA was used as template DNA for the PCR reactions. The template DNA, 12.5 pmol or 40 pmol (CY5-labelled) of each primer, 0.5-2 U Taq polymerase (HotStarTaq, Qiagen, Hilden, Germany) and 1 mM dNTPs were incubated with the reaction buffer supplied with the enzyme in a total volume of 20 μl. After activation of the enzyme (15 min, 96° C.) the incubation times and temperatures were 95° C. for 1 min followed by 34 cycles (95° C. for 1 min, annealing temperature (see Supplementary information) for 45 sec, 72° C. for 75 sec) and 72° C. for 10 min.

See Table 2 for further details of PCR products.

All PCR products from each individual sample were then hybridised to glass slides carrying a pair of immobilised oligonucleotides for each CpG position under analysis. Each of these detection oligonucleotides was designed to hybridise to the bisulphite converted sequence around one CpG site which was either originally unmethylated (TG) or methylated (CG). See Table 3 for further details of all hybridisation oligonucleotides used (both informative and non-informative) Hybridisation conditions were selected to allow the detection of the single nucleotide differences between the TG and CG variants.

Oligonucleotides with a C6-amino modification at the 5′end were spotted with 4-fold redundancy on activated glass slides (T. R. Golub et al., Science 286, 531 (1999)). For each analysed CpG position two oligonucleotides N₍₂₋₁₆₎-CG-N₍₂₋₁₆₎ and N₍₂₋₁₆₎-TG-N₍₂₋₁₆₎, reflecting the methylated and non methylated status of the CpG dinucleotides, were spotted and immobilised on the glass array. The oligonucleotide microarrays representing 81 CpG sites were hybridised with a combination of up to 11 Cy5-labelled PCR fragments as described earlier (D. Chen, Z. Yan, D. L. Cole, G. S. Srivatsa, Nucleic Acid Res 27, 389 (1999)). Subsequently, the fluorescent images of the hybridised slides were obtained using a GenePix 4000 microarray scanner (Axon Instruments). Hybridisation experiments were repeated at least three times for each sample.

The sensitivity of the method for detection of methylation changes was determined using artificially up- and downmethylated DNA fragments mixed at different ratios. For each of those mixtures, a series of experiments was conducted to define the range of CG/TG ratios that corresponds to varying degrees of methylation at each of the CpG sites tested. In FIG. 1B results for two CpG positions located in exon 14 of the human factor VIII gene are shown as examples. For the mixtures of 3:0, 2:1, 1:2 and 0:3 the degree of methylation of the individual CpG sites could safely be distinguished.

To verify the detection of methylation changes in the real data set two X-chromosomal genes were included in the gene set. Because one of the two X-chromosomes in females becomes inactivated by methylation we can expect a higher degree of methylation of X-chromosomal genes in females compared to males. In FIG. 2A CpGs are ranked according to the significance of the difference between male and female methylation levels. As expected, the X-chromosomal genes (ELK1, AR) show a significantly higher methylation for females. This clearly demonstrates that the method really detects changes in methylation.

### Statistical Analysis

### Support Vector Machines

In our case, the task of cancer classification consists of constructing a machine that can predict the leukemia subtype (ALL or AML) from a patients methylation pattern. For every patient sample this pattern is given as a vector of average (Every hybridisation experiment was at least 3 times repeated and the results averaged.) log CG/TG ratios at 81 CpG positions. Based on a given set of training examples X={xⁱ:xⁱ i Rⁿ} with known diagnosis Y={yⁱ:yⁱ i {ALL,AML}} a discriminant function f: Rⁿ ® {ALL,AML}, where n is the number of CpGs, has to be learned. The number of misclassifications of f on the training set {X,Y} is called training error and is usually minimised by the learning machine during the training phase. However, what is of practical interest is the capability to predict the class of previously unseen samples, the so called generalisation performance of the learning machine. This performance is usually estimated by the test error, which is the number of misclassifications on an independent test set {X′,Y′}.

The major problem of training a learning machine with good generalisation performance is to find a discriminant function f which on the one hand is complex enough to capture the essential properties of the data distribution, but which on the other hand avoids over-fitting the data. The Support Vector Machine (SVM) tries to solve this problem by constructing a linear discriminant that separates the training data and maximises the distance to the nearest points of the training set. This maximum margin separating hyperplane minimises the ratio between the radius of the minimum enclosing sphere of the training set and the margin between hyperplane and training points. This corresponds to minimising the so called radius margin bound on the expected probability of a test error and promises good generalisation performance (Vapnik V. “Statistical Learning Theory.” Wiley, New York (1998)).

Of course there are more complex classification problems, where the dependence between class labels yⁱ and features xⁱ is not linear and the training set can not be separated by a hyperplane. In order to allow for non-linear discriminant functions the input space can be non-linearly mapped into a potentially higher dimensional feature space by a mapping function F: xⁱ ® F(xⁱ). Because the SVM algorithm in its dual formulation uses only the inner product between elements of the input space, the knowledge of the kernel function K(xⁱ, x^{j})=<F(xⁱ)·F(x^{j})> is sufficient to train the SVM. It is not necessary to explicitly know the mapping F and a non-linear SVM can be trained efficiently by computing only the kernel function. Here we will only use the linear kernel K(xⁱ, x^{j})=<x^{i,} x^{j}> and the quadratic kernel K(xⁱ, x^{j})=(<x^{i,xj}>+1)₂. In the next section we will compare SVMs trained on different feature sets. In order to evaluate the prediction performance of these SVMs we used a cross-validation method (Bishop, C. M. “Neural networks for pattern recognition.” Oxford University Press, New York (1995)). For each classification task, the samples were partitioned into 8 groups of approximately equal size. Then the SVM predicted the class for the test samples in one group after it had been trained using the 7 other groups. The number of misclassifications was counted over 8 runs of the SVM algorithm for all possible choices of the test group. To obtain a reliable estimate for the test error the number of misclassifications were averaged over 50 different partitionings of the samples into 8 groups.

### Feature Selection

The simplest way for applying a SVM to our methylation data is to use every CpG position as a separate dimension, not making any assumption about the interdependence of CpG sites from the same gene. On the leukemia subclassification task the SVM with linear kernel trained on this 81 dimensional input space had an average test error of 16%. Using a quadratic kernel did not significantly improve the results.

An obvious explanation for this relatively poor performance is that we have only 25 data points (even less in the training set) in a 81 dimensional space. Finding a separating hyperplane under these conditions is a heavily under-determined problem. And as it turns out, the SVM technique of maximising the margin is not sufficient to find the solution with optimal generalisation properties. It is necessary to reduce the dimensionality of the input space while retaining the relevant information for classification. This should be possible because it can be expected that only a minority of CpG positions has any connection with the two subtypes of leukemia.

### Principle Component Analysis

The probably most popular method for dimension reduction is principle component analysis (PCA) (Bishop, C. M. “Neural networks for pattern recognition.” Oxford University Press, New York (1995)). For a given training set X, PCA constructs a set of orthogonal vectors (principle components) which correspond to the directions of maximum variance. The projection of X onto the first k principle components gives the 2-norm optimal representation of X in a k-dimensional orthogonal subspace. Because this projection does not explicitly use the class information Y, PCA is an unsupervised learning technique.

In order to reduce the dimension of the input space for the SVM we performed a PCA on the combined training and test set {X,X′} and projected both sets on the first k principle components. This gives considerably better results than performing PCA only on the training set X and is justified by the fact that no label information is used. However, the generalisation results for k=2 and k=5, were even worse than for the SVM without feature selection. The reason for this is that PCA does not necessarily extract features that are important for the discrimination between ALL and AML. It first picks the features with the highest variance, which are in this case discriminating between cell lines and primary patient tissue (see FIG. 4, right, second line), i.e. subgroups that are not relevant to the classification task. As is shown in FIG. 6, features carrying information about the leukemia subclasses appear only from the 9th principle component on. The generalisation performance including the 9th component is significantly better than for a SVM without feature selection. However, it seems clear that a supervised feature selection method, which takes the class labels of the training set into account, should be more reliable and give better generalisation.

FIG. 4A shows the methylation profiles of the best 20 CpGs according to the Fisher criterion.

### Fisher Criterion and t-Test

A classical measure to asses the degree of separation between two classes is given by the Fisher criterion cite (Bishop, C. M. “Neural networks for pattern recognition.” Oxford University Press, New York (1995)). In our case it gives the discriminative power of the kth CpG as

where (mₖ)^{ALL/AML} is the mean and (sₖ)^{ALL/AML} is the standard deviation of all (′ₖ)ⁱ with yᵢ=ALL/AML. The Fisher criterion gives a high ranking for CpGs where the two classes are far apart compared to the within class variances. A very similar criterion was used by Golub and coworkers for their ALL/AML classification based on mRNA expression data (T. R. Golub et al., Science 286, 531 (1999)). FIG. 4B hows the methylation profiles of the best 20 CpGs according to the Fisher criterion.

Another approach to rank CpGs by their discriminative power is to use a test statistic for computing the significance of class differences. Here we assumed normal distribution of the two classes and used a two sample t-test to rank the CpGs according to the significance of the difference between the class means (Mendenhall, W., Sincich, T., “Statistics for engineering and the sciences.” FIG. 4 (right, first line) shows the ranking, which is very similar to the Fisher criterion because a large mean difference and a small within class variance are again the important factors.

In order to improve classification performance we trained SVMs on the k highest ranking CpGs according to the Fisher criterion or t-test. FIG. 5 shows a trained SVM on the best two CpGs from the Fisher criterion. The test errors for k=2 and k=5 are given in the table above. The results show a dramatic improvement of generalisation performance. Using Fisher criterion for feature selection and k=5 CpGs the test error was decreased to 3% compared to 16% for the SVM without feature selection. FIG. 6 shows the dependence of generalisation performance from the selected dimension k and indicates that especially the Fisher criterion gives dimension independent good generalisation for reasonable small k.

Although the two described CpG ranking methods give very good generalisation, they have some potential drawbacks. One problem is that they can only detect linear dependencies between features and class labels. A simple XOR or even OR combination of two CpGs would be completely missed. Another drawback is that redundant features are not removed. In our case there are usually several CpGs from the same gene which have a high likelihood of comethylation. This can result in a large set of high ranking features which carry essentially the same information. Although the good results seem to indicate that the described problems do not appear in our data set, they should be considered.

### Backward Elimination

PCA, Fisher criterion and t-test construct or rank features independent of the leaning machine that does the actual classification and are therefore called filter methods (Blum, A., Langley, P. “Selection of relevant features and examples in machine learning.” Artificial Intelligence 97, 245-271 (1997)). Another approach is to use the learning machine itself for feature selection. These techniques are called wrapper methods and try to identify the features that are important for the generalisation capability of the machine. Here we propose to use the features that are important for achieving a low training error as a simple approximation. In the case of a SVM with linear kernel these features are easily identified by looking at the normal vector w of the separating hyperplane. The smaller the angle between a feature basis vector and the normal vector the more important is the feature for the separation. Features orthogonal to the normal vector have obviously no influence on the discrimination at all. This means the feature ranking is simply given by the components of the normal vector as (wₖ₎². Of course this ranking is not very realistic because the SVM solution on the full feature set is far from optimal as we demonstrated in the last subsections. A simple heuristic is to assume that the feature with the smallest (wₖ)² is really unimportant for the solution and can be safely removed from the feature set.

Then the SVM can be retrained on the reduced feature set and the procedure is repeated until the feature set is empty. Such a successive feature removal is called backward elimination (Blum, A., Langley, P. “Selection of relevant features and examples in machine learning.” Artificial Intelligence 97, 245-271 (1997)). The resulting CpG ranking on our data set is shown in FIG. 4D and differs considerably from the Fisher and t-test rankings. It seems backward elimination is able to remove redundant features. However, as shown in the table above and FIG. 6 the generalisation results are not better than for the Fisher criterion. Furthermore, backward elimination seems to be more dimension dependent and it is computationally more expensive. It follows that at least for this data set the simple Fisher criterion is the preferable feature selection technique.

### Exhaustive Search

A canonical way to construct a wrapper method for feature selection is to evaluate the generalisation performance of the learning machine on every possible feature subset. Cross-validation on the training set can be used to estimate the generalisation of the machine on a given feature set. What makes this exhaustive search of the feature space practically useless is the enormous number of 2ⁿ different feature combinations and there are numerous heuristics to search the feature space more efficiently (e.g. backward elimination) (Blum, A., Langley, P. “Selection of relevant features and examples in machine learning.” Artificial Intelligence 97, 245-271 (1997)).

Here we only want to demonstrate that there are no higher order correlations between features and class labels in our data set In order to do this we exhaustively searched the space of all two feature combinations. For every of the 3240 two CpG combinations of k=2 CpGs at the same time without repetitions, out of all together n=81 CpGs we computed the leave-one-out cross-validation error of a SVM with quadratic kernel on the training set. From all CpG pairs with minimum leave-one-out error we selected the one with the smallest radius margin ratio. This pair was considered to be the optimal feature combination and was used to evaluate the generalisation performance of the SVM on the test set.

The average test error of the exhaustive search method was with 6% the same as the one of the Fisher criterion in the case of two features and a quadratic kernel. For five features the exhaustive computation is already infeasible. In the absolute majority of cross-validation runs the CpGs selected by exhaustive search and Fisher criterion were identical. In some cases suboptimal CpGs were chosen by the exhaustive search method. These results clearly demonstrate that there are no higher order feature combinations in our data set that are important for an ALL/AML discrimination.

### EXAMPLE 2

The data obtained according to Example 1 is sorted into a ranked matrix (FIGS. 3 to 8) according to CpG methylation differences between the two classes of diseased and healthy hematopoietic cells using an algorithim. The most significant CpG positions are at the bottom of the matrix with significance decreasing towards the top. On the right side of the matrix p values for the individual CpG positions are shown. The p values are the probabilities that the observed distribution occurred by chance in the data set.

As discussed above, for selected distinctions, we trained a learning algorithm (support vector machine, SVM). The SVM (as discussed by F. Model, P. Adorjan, A. Olek, C. Piepenbrock, Feature selection for DNA methylation based cancer classification. Bioinformatics. 2001 June;17 Suppl 1:S157-64) constructs an optimal discriminant between two classes of given training samples. In this case each sample is described by the methylation patterns (CG/TG ratios) at the investigated CpG sites. The SVM was trained on a subset of samples of each class, which were presented with the diagnosis attached. Independent test samples, which were not shown to the SVM before were then presented to evaluate, if the diagnosis can be predicted correctly based on the predictor created in the training round.

A cross validation method was used to evaluate the prediction performance of the SVM (C. M. Bishop, Neural networks for pattern recognition (Oxford University Press, New York 1995)). For each classification task, the samples were partitioned into 8 groups of approximately equal size. Then the SVM predicted the class for the test samples in one group after it had been trained using the 7 other groups. The number of misclassifications was counted over 8 runs of the SVM algorithm for all possible choices of the test group. To obtain a reliable estimate for the test error, i.e. the probability of misclassification for a previously unknown sample, the number of misclassifications were averaged over 10 different partitionings of the samples into 8 groups.

The SVM was trained to recognise the difference between healthy CD 19+ B cells and CD4+ T cells, and T and B cell leukaemias (from both patient samples and cell lines). Samples could be classified with 15% test error using the two most informative CpG positions. Remarkably, the test error could be reduced to 4% by including a total of 54 CpG positions into the analysis. Individual CpG sites were ranked according to their contribution to the decision of the support vector machine, showing that the decision between healthy T and B cells and ALL was primarily based on CpG sites located in intron 1 of the CDK4 gene and the coding sequence of the c-MOS oncogene, but CpG sites located in regulatory regions of other genes contributed significantly (Table 1 and FIGS. 2B and 2C). Compared to the healthy group, the tumour cells consistently showed relative hypermethylation of these particular CpG sites. Also, classification could be achieved between ALL patient samples and healthy donor B and T cells with a test error of only 13% using two CpG positions. This low-dimensional classification was based on methylation status of CpG sites from CSNK2B and CDC25A, both of which were hypermethylated in ALL patients. The test error could be further improved to 5% by increasing the number of CpG positions used for classification to a total of 31.

In a next step, we tried to distinguish the two classes of leukaemia. Again the support vector machine was initially presented with a training set of AML and ALL samples (from both patient samples and cell lines) with the class information attached. Previously not included samples could then be classified into one of the classes with a test error of 5% when using two CpG positions. The optimal number of CpG sites in this case was calculated to be 6, decreasing the test error to only 1% (Table 1 and FIG. 3B). The most informative CpG sites were located in intron 1 of the CDK4 gene and the promoter region of CSNK2B. The overall degree of methylation at these sites was reproducibly higher in ALL than in AML cells. However, the CpG sites in the CDK4 gene contributing information to this particular decision were different from those in the same gene distinguishing between ALL and healthy lymphocytes. This shows clearly that in some cases, different CpG sites within one cluster can contribute independent information, with different CpG sites potentially answering questions on different aspects of a phenotype.

There is evidence that CpG islands of several tissue-specific genes become methylated in cell lines (F. Antequera, J. Boyes, A. Bird, Cell 62, 503 (1990)). Hence, it is important to rule out that the above classifications are based on cell line specific artefacts rather than on disease specific changes. We therefore grouped leukaemia cell lines in one and primary leukaemia samples into another class. Classification was possible with at test error of 28% based on 2 CpG positions and 12% using 30 CpG sites (Table 1). Importantly, the CpG sites that contributed most to this distinction were not informative in any of the earlier classifications, excluding that any of them is based merely on cell line specific markers. This knowledge then enabled us to identify disease specific methylation differences between healthy T cells and T-ALL cell lines. Classification was performed with a test error of 0% based on two CpG positions, which could not distinguish cell lines from non cell lines. Decisive CpG sites were located in the coding sequence of the c-MOS gene (Table 1). In conclusion, although our results confirm the occurrence of cell line specific methylation changes, these do not interfere with classification of tumour types.

FIG. 7 shows the use of a selection of genes from the panel for use in differentiating between acute lymphocytic leukemia and acute myelogenous leukemia. Using the 2 most significant CpG positions, test error was calculated to be 5%. The optimal number of CpG sites in this case was calculated to be 6, decreasing the test error to 1%. The most informative CpG sites were located in intron 1 of the CDK4 gene and the promoter region of CSNK2B. The overall degree of methylation at these sites was reproducibly higher in ALL than in AML cells. However, the CpG sites in the CDK4 gene contributing information to this particular decision were different from those in the same gene distinguishing between ALL and healthy lymphocytes. This shows clearly that in some cases, different CpG sites within one cluster can contribute independent information, with different CpG sites potentially answering questions on different aspects of a phenotype.

FIG. 8 shows the use of an alternative selection of genes from the panel for use in differentiating between acute lymphocytic leukemia and acute myelogenous leukemia. The test error in this case was 16.3%.

In all classes classification results were comparable to mRNA based assays regarding test error, safety and reproducibility. However, classification was achieved with significantly fewer, randomly chosen genes, suggesting that methylation data may actually contain more information per dimension than expression profiles.

More importantly, in expression profiling signal intensities strongly depend on both the absolute and relative amounts of the different mRNA-species, comparison between independent experiments is challenging at best. Hence, although signals are generally calibrated against those derived from housekeeping genes, there remains an inability of mRNA profiling to detect subtle changes in expression levels (R. J. Lipshutz, S. P. A Fodor, T. R. Gingeras, D. J. Lockhart, Nature Genetics 21, 20 (1999)). In our approach, a CG/TG ratio is used as an internal calibration, and thus the amount of probe hybridised to the slide does not influence the results. This greatly improves the comparability of the results and therefore enables the screening of larger populations, as for example needed in multicenter trials and prospective studies.

### EXAMPLE 3

### Identification of the methylation status of a CpG site within the gene N33 (NM_{—}006765).

A fragment of the gene N33 (Seq ID NO: &[GENEID_{—}2188]) was PCR amplified using primers AAAGCCGCTGCCATCC and TTTCGGCGACGGTAGG. The resultant fragment (548 bp in length) contained an informative CpG at position 446. The amplificate DNA was digested with the restriction endonuclease Faul, recogniton site CCCGC. Hydrolysis by said endonuclease is blocked by methylation of the CpG at position 446 of the amplificate. The digest was used as a control.

Genomic DNA was isolated from sample using the DNA wizzard DNA isolation kit (Promega). Each sample was digested using AvaI according to manufacturer's recommendations (New England Biolabs).

10 ng of each genomic digest was then amplified using PCR primers AAAGCCGCTGCCATCC and TTTCGGCGACGGTAGG. The PCR reactions were performed using a thermocycler (Eppendorf GmbH) using 10 ng of DNA, 6 pmole of each primer, 200 μM of each dNTP, 1.5 mM MgCl2 and 1 U of HotstartTaq (Qiagen AG). The other conditions were as recommended by the Taq polymerase manufacturer. Using the above mentioned primers, gene fragments were amplified by PCR performing a first denaturation step for 14 min at 96° C., followed by 30-45 cycles (step 2: 60 sec at 96° C., step 3: 45 sec at 52° C., step 4: 75 sec at 72° C.) and a subsequent final elongation of 10 min at 72° C. The presence of PCR products was analysed by agrarose gel electrophoresis.

PCR products were detectable with FauI hydrolyzed DNA isolated wherein the CpG position in question was upmethylated, when step 2 to step 4 of the cycle program were repeated 34, 37, 39, 42 and 45 fold. In contrast PCR products were only detectable with Faul hydrolyzed DNA isolated from downmethylated DNA (and control DNA) when step 2 to step 4 of the cycle program were repeated 39, 42 and 45 fold. These results were incorporated into a CpG methylation matrix analysis as described in Example 2.

### Tables

TABLE 1

| | | | | Test Error | Significance | Optimal | Test Error | Significance |
|---|---|---|---|---|---|---|---|---|
| Class 1 | | Class 2 | | 2 CpGs | 2 CpGs | CpG Number | optimal CpGs | optimal CpGs |
| Healthy-B | 13 | B-ALL-cl | 17 | 15% | 1.3E−4 | 54 | 4% | 3.2E−7 |
| Healthy-T | | T-ALL-cl | | | | | | |
| | | ALL | | | | | | |
| B-ALL-cl | 17 | AML | 8 | 5% | 3.7E−4 | 6 | 1% | 5.4E−10 |
| T-ALL-cl | | AML-cl | | | | | | |
| ALL | | | | | | | | |
| Female-nocl | 14 | Male-nocl | 7 | 5% | 7.4E−4 | 2 | 5% | 7.4E−4 |
| Healthy-T | 6 | T-ALL-cl | 6 | 0% | 2.5E−3 | 2 | 0% | 2.5E−3 |
| Healthy-B | 13 | AML | 8 | 16% | 6.9E−3 | 22 | 0% | 2.3E−6 |
| Healthy-T | | AML-cl | | | | | | |
| Primary leukaemia | 8 | CellLines | 17 | 28% | 2.2E−2 | 30 | 12% | 7.9E−5 |
| samples | | | | | | | | |
| Healthy-B | 13 | ALL | 5 | 13% | 8.2E−3 | 31 | 5% | 9.1E−5 |
| Healthy-T | | | | | | | | |
| Healthy-B | 7 | B-ALL-cl | 6 | 27% | 5.5E−2 | 11 | 8% | 1.8E−3 |
| Female-cl | 9 | Male-cl | 8 | 52% | 5.3E−1 | 42 | 40% | 1.9E−1 |
| B-ALL-cl | 6 | T-ALL-cl | 6 | 58% | 5.7E−1 | 70 | 51% | 3.9E−1 |
| Healthy-B | 7 | Healthy-T | 6 | 63% | 6.8E−1 | 11 | 49% | 3.8E−1 |

TABLE 2

PCR primers and products

Amplificate

| | | | |
|---|---|---|---|
| No: | Gene: | Primer: | Length: |

| | | | |
|---|---|---|---|
| 1 | MDR1 | TAAGTATGTTGAAGAAAGATTATTGTAG | 633 |
| | (SEQ ID NO: 37) | (SEQ ID NO: 388) | |
| | | TAAAAACTATCCCATAATAACTCCCAAC | |
| | | (SEQ ID NO: 387) | |
| 2 | CSNK2B | GGGGAAATGGAGAAGTGTAA | 524 |
| | (SEQ ID NO: 67) | (SEQ ID NO: 512) | |
| | | CTACCAATCCGAAAATAACC | |
| | | (SEQ ID NO: 511) | |
| 3 | EGR4 | AGGGGGATTGAGTGTTAAGT | 293 |
| | (SEQ ID NO: 26) | (SEQ ID NO: 392) | |
| | | CCCAAACATAAACACAAAAT | |
| | | (SEQ ID NO: 391) | |
| 4 | AR | GTAGTAGTAGTAGTAAGAGA | 460 |
| | (SEQ ID NO: 69) | (SEQ ID NO: 532) | |
| | | ACCCCCTAAATAATTATCCT | |
| | | (SEQ ID NO: 531) | |
| 5 | CDK 4 | GGTAGTTGGTTATATGGTGAGG | 748 |
| | (SEQ ID NO: 70) | (SEQ ID NO: 395) | |
| | | TCACACTCTTTAAAAACCACAAAA | |
| | | (SEQ ID NO: 396) | |
| 6 | Humos | TGATTGGGAGTAGGTGTGTT | 523 |
| | (SEQ ID NO: 71) | (SEQ ID NO: 397) | |
| | | CAAATCTTCCAACTTCTCAAA | |
| | | (SEQ ID NO: 398) | |
| 7 | RB1 | TTTAAGTTTGTTTTTGTTTTGGT | 719 |
| | (SEQ ID NO: 52) | (SEQ ID NO: 399) | |
| | | TCCTACTCTAAATCCTCCTCAA | |
| | | (SEQ ID NO: 400) | |
| 8 | CDC25A | TTGGGAGTTTTTATTGATTTTT | 445 |
| | (SEQ ID NO: 72) | (SEQ ID NO: 401) | |
| | | ACAACCTAAAAATTAAATCCAAA | |
| | | (SEQ ID NO: 402) | |
| 9 | GPIb beta | GGTGATAGGAGAATAATGTTGG | 379 |
| | (SEQ ID NO: 30) | (SEQ ID NO: 403) | |
| | | TCTCCCAACTACAACCAAAC | |
| | | (SEQ ID NO: 404) | |
| 10 | MYOD1 | ATTAGGGGTATAGAGGAGTATTGA | 883 |
| | (SEQ ID NO: 45) | (SEQ ID NO: 405) | |
| | | CTTACAAACCCACAATAAACAA | |
| | | (SEQ ID NO: 406) | |
| 11 | CDH3 | GTTTAGAAGTTTAAGATTAG | 611 |
| | (SEQ ID NO: 17) | (SEQ ID NO: 407) | |
| | | CAAAAACTCAACCTCTATCT | |
| | | (SEQ ID NO: 408) | |
| 12 | WT1 | AAAGGGAAATTAAGTGTTGT | 747 |
| | (SEQ ID NO: 59) | (SEQ ID NO: 410) | |
| | | TAACTACCCTCAACTTCCC | |
| | | (SEQ ID NO: 409) | |
| 13 | MYCL1 | AGGTTTGGGTTATTGAGTTT | 491 |
| | (SEQ ID NO: 44) | (SEQ ID NO: 411) | |
| | | CATTATTTCCTAACTACCTTATATCTC | |
| | | (SEQ ID NO: 412) | |
| 14 | ABL1 | GGTTGGGAGATTTAATTTTATT | 967 |
| | (SEQ ID NO: 2) | (SEQ ID NO: 414) | |
| | | ACCAATCCAAACTTTTTCCTT | |
| | | (SEQ ID NO: 413) | |
| 15 | ELK1 | AAGTGTTTTAGTTTTTAATGGGTA | 966 |
| | (SEQ ID NO: 27) | (SEQ ID NO: 415) | |
| | | CAAACCCAAAACTCACCTAT | |
| | | (SEQ ID NO: 416) | |
| 16 | ABL1 | GTTAGGAGGGGGTTAAGG | 291 |
| | (SEQ ID NO: 1) | (SEQ ID NO: 417) | |
| | | CCAACTTCAAACAAATCTCC | |
| | | (SEQ ID NO: 418) | |
| 17 | APC | TCAACTACCATCAACTTCCTTA | |
| | (SEQ ID NO: 4) | (SEQ ID NO: 419) | |
| | | AATTTATTTTTAGTGTTGTAGTGGG | |
| | | (SEQ ID NO: 420) | |
| 18 | ARHI | GTGAGTTTTTGGGGTGTTTA | 444 |
| | (SEQ ID NO: 6) | (SEQ ID NO: 421) | |
| | | TCAATCTTACTTTCACACTACATAA | |
| | | (SEQ ID NO: 422) | |
| 19 | BCL2 | GTATTTTATGTTAAGGGGGAAA | 640 |
| | (SEQ ID NO: 9) | (SEQ ID NO: 423) | |
| | | AAAAACCACAATCCTCCC | |
| | | (SEQ ID NO: 424) | |
| 20 | CCND2 | TTTTGGTATGTAGGTTGGATG | 426 |
| | (SEQ ID NO: 13) | (SEQ ID NO: 425) | |
| | | CCTAACCTCCTTCCTTTAACT | |
| | | (SEQ ID NO: 426) | |
| 21 | CDH1 | CAAATAAACCCTCAACCAATC | 474 |
| | (SEQ ID NO: 16) | (SEQ ID NO: 427) | |
| | | TGGAGGGGGTAGGAAAGT | |
| | | (SEQ ID NO: 428) | |
| 22 | CDKN1A | GGATTAGTGGGAATAGAGGTG | 408 |
| | (SEQ ID NO: 19) | (SEQ ID NO: 429) | |
| | | AAACCCAAACTCCTAACTACC | |
| | | (SEQ ID NO: 430) | |
| 23 | CDKN1B | GTGGGGAGGTAGTTGAAGA | 478 |
| | (p27 Kip1) | (SEQ ID NO: 431) | |
| | (SEQ ID NO: 20) | ATACACCCCTAACCCAAAAT | |
| | | (SEQ ID NO: 432) | |
| 24 | CDKN2a | TTGAAAATTAAGGGTTGAGG | 598 |
| | (SEQ ID NO: 22) | (SEQ ID NO: 433) | |
| | | CACCCTCTAATAACCAACCA | |
| | | (SEQ ID NO: 434) | |
| 25 | CDKN2a | GGGGTTGGTTGGTTATTAGA | 256 |
| | (SEQ ID NO: 22) | (SEQ ID NO: 435) | |
| | | AACCCTCTACCCACCTAAAT | |
| | | (SEQ ID NO: 436) | |
| 26 | CDKN2B | GGTTGGTTGAAGGAATAGAAAT | 708 |
| | (SEQ ID NO: 23) | (SEQ ID NO: 437) | |
| | | CCCACTAAACATACCCTTATTC | |
| | | (SEQ ID NO: 438) | |
| 27 | DAPK1 | AACCCTTTCTTCAAATTACAAA | 348 |
| | (SEQ ID NO: 25) | (SEQ ID NO: 439) | |
| | | TGATTGGGTTTTAGGGAAATA | |
| | | (SEQ ID NO: 440) | |
| 28 | FOS | TTTTTGGGGTTTAGTTTAGAAT | 308 |
| | (SEQ ID NO: 29) | (SEQ ID NO: 441) | |
| | | AACCTTCATCCCCTAACCT | |
| | | (SEQ ID NO: 442) | |
| 29 | GSTP1 | ATTTGGGAAAGAGGGAAAG | 300 |
| | (SEQ ID NO: 33) | (SEQ ID NO: 443) | |
| | | TAAAAACTCTAAACCCCATCC | |
| | | (SEQ ID NO: 444) | |
| 30 | HIC-1 | TGGGTTGGAGAAGAAGTTTA | 280 |
| | (SEQ ID NO: 34) | (SEQ ID NO: 445) | |
| | | TCATATTTCCAAAAACACACC | |
| | | (SEQ ID NO: 446) | |
| 31 | IGF2 | CCCTTCCCCTTAACTAAACT | 364 |
| | (SEQ ID NO: 36) | (SEQ ID NO: 447) | |
| | | AATTTGGGTTAGGTTTGGA | |
| | | (SEQ ID NO: 448) | |
| 32 | MGMT | AAGGTTTTAGGGAAGAGTGTTT | 636 |
| | (SEQ ID NO: 38) | (SEQ ID NO: 449) | |
| | | ACCTTTTCCTATCACAAAAATAA | |
| | | (SEQ ID NO: 450) | |
| 33 | MLH1 | TAAGGGGAGAGGAGGAGTTT | 545 |
| | (SEQ ID NO: 39) | (SEQ ID NO: 451) | |
| | | ACCAATTCTCAATCATCTCTTT | |
| | | (SEQ ID NO: 452) | |
| 34 | MOS | ACCCTACAACAATCCCTCA | 343 |
| | (SEQ ID NO: 40) | (SEQ ID NO: 453) | |
| | | TGGTTTTTAGGTTATTGGATTT | |
| | | (SEQ ID NO: 454) | |
| 35 | MPL | TGGGGAGATTGATTTGAGTAT | 594 |
| | (SEQ ID NO: 42) | (SEQ ID NO: 455) | |
| | | AATTCCTTCAATAACATACCCTT | |
| | | (SEQ ID NO: 456) | |
| 36 | MYC | AGAGGGAGTAAAAGAAAATGGT | 712 |
| | (SEQ ID NO: 43) | (SEQ ID NO: 457) | |
| | | CCAAATAAACAAAATAACCTCC | |
| | | (SEQ ID NO: 458) | |
| 37 | N33 | TTTTAGATTGAGGTTTTAGGGT | 498 |
| | (SEQ ID NO: 46) | (SEQ ID NO: 459) | |
| | | ATCCATTCTACCTCCTTTTTCT | |
| | | (SEQ ID NO: 460) | |
| 38 | PMS2 | GATTGAGATTATTTTGGGTTTT | 561 |
| | (SEQ ID NO: 49) | (SEQ ID NO: 461) | |
| | | ACTTAAACCTTCCCTCTCCAC | |
| | | (SEQ ID NO: 462) | |
| 39 | PTEN | TTTTAGGTAGTTATATTGGGTATGTT | 346 |
| | (SEQ ID NO: 51) | (SEQ ID NO: 463) | |
| | | TCAACTCTCAAACTTCCATCA | |
| | | (SEQ ID NO: 464) | |
| 40 | RBL2 | GAAAATGGGTGTGTGTGG | 112 |
| | (SEQ ID NO: 53) | (SEQ ID NO: 465) | |
| | | TACAAATAAAAACAAATCCCCT | |
| | | (SEQ ID NO: 466) | |
| 41 | SFN | GAAGAGAGGAGAGGGAGGTA | 489 |
| | (SEQ ID NO: 55) | (SEQ ID NO: 468) | |
| | | CTATCCAACAAACCCAACA | |
| | | (SEQ ID NO: 467) | |
| 42 | TGFBR2 | GTAATTTGAAGAAAGTTGAGGG | 296 |
| | (SEQ ID NO: 57) | (SEQ ID NO: 469) | |
| | | CCAACAACTAAACAAAACCTCT | |
| | | (SEQ ID NO: 470) | |
| 43 | TP73 | AGTAAATAGTGGGTGAGTTATGAA | 607 |
| | (SEQ ID NO: 58) | (SEQ ID NO: 472) | |
| | | GAAAAACCTCTAAAAACTACTCTCC | |
| | | (SEQ ID NO: 471) | |
| 44 | CDKN1C | GGGGAGGTAGATATTTGGATAA | 300 |
| | (SEQ ID NO: 21) | (SEQ ID NO: 473) | |
| | | AACTACACCATTTATATTCCCAC | |
| | | (SEQ ID NO: 474) | |
| 45 | GSK3β | TAAGTGATAAAGGAAGGAAGGA | 243 |
| | (SEQ ID NO: 32) | (SEQ ID NO: 475) | |
| | | CCTTCAAACCCCAAACAA | |
| | | (SEQ ID NO: 476) | |
| 46 | CDC2 | ATTAGAAGTGAAAGTAATGGAATTT | 418 |
| | (SEQ ID NO: 14) | (SEQ ID NO: 477) | |
| | | TCAATTTCCAAAAACCAAC | |
| | | (SEQ ID NO: 478) | |
| 47 | ESR1 | AGGGGGAATTAAATAGAAAGAG | 662 |
| | (SEQ ID NO: 28) | (SEQ ID NO: 479) | |
| | | CAATAAAACCATCCCAAATACT | |
| | | (SEQ ID NO: 480) | |
| 48 | APAF1 | AGATATGTTTGGAGATTTTAGGA | 674 |
| | (SEQ ID NO: 3) | (SEQ ID NO: 481) | |
| | | AACTCCCCACCTCTAATTCTAT | |
| | | (SEQ ID NO: 482) | |
| 49 | BAK1 | AATTAGGGATGGGAAAAGTAGT | 558 |
| | (SEQ ID NO: 7) | (SEQ ID NO: 483) | |
| | | AAACATAACAAAATCAAATCCC | |
| | | (SEQ ID NO: 484) | |
| 50 | BAX | AAATAAATAGAAAAGTAGGTTTGGC | 716 |
| | (SEQ ID NO: 8) | (SEQ ID NO: 485) | |
| | | TTCTACCCCTCAATACTTAAAAA | |
| | | (SEQ ID NO: 486) | |
| 51 | CASP10 | TCTTCCCAAACAAATAAAAACT | 468 |
| | (SEQ ID NO: 10) | (SEQ ID NO: 487) | |
| | | GTTTTGAGGTTAAATGAGTGGT | |
| | | (SEQ ID NO: 488) | |
| 52 | CASP8 | AGTGGATTTGGAGTTTAGATGT | 431 |
| | (SEQ ID NO: 11) | (SEQ ID NO: 489) | |
| | | AACAAAATAAAAACTTGTCCCA | |
| | | (SEQ ID NO: 490) | |
| 53 | CASP9 | GGAAGAGTTGTAGGTGGATTAG | 424 |
| | (SEQ ID NO: 12) | (SEQ ID NO: 491) | |
| | | CCACATTTTCCCAATAAATACT | |
| | | (SEQ ID NO: 492) | |
| 54 | TCL1A | CCACAACACATCAAACCTATAA | 491 |
| | (SEQ ID NO: 56) | (SEQ ID NO: 493) | |
| | | TTTGGTGGTAAGTGAGGGT | |
| | | (SEQ ID NO: 494) | |
| 55 | PML | AATTCCCAAACAAACTTTTACA | 459 |
| | (SEQ ID NO: 48) | (SEQ ID NO: 495) | |
| | | TGGAGAGGAAGTGAGATAGAAG | |
| | | (SEQ ID NO: 496) | |
| 56 | HOXA5 | AAACCCCAAACAACCTCTAT | 392 |
| | (SEQ ID NO: 35) | (SEQ ID NO: 498) | |
| | | GAAGGGGGAAAGTTATTTAGTTA | |
| | | (SEQ ID NO: 497) | |
| 57 | SDC4 | CCTAACTACCCTCATTCCTTT | 269 |
| | (SEQ ID NO: 54) | (SEQ ID NO: 499) | |
| | | AGTTGGGGAAATTAAGGTTTAG | |
| | | (SEQ ID NO: 500) | |
| 58 | PITX2 | TCCTCAACTCTACAAACCTAAAA | 408 |
| | (SEQ ID NO: 47) | (SEQ ID NO: 501) | |
| | | GTAGGGGAGGGAAGTAGATGT | |
| | | (SEQ ID NO: 502) | |
| 59 | GPR37 | ACTTATTTTTCTTTTCCTCTAAAAAC | 489 |
| | (SEQ ID NO: 31) | (SEQ ID NO: 503) | |
| | | TATGGTTTGGTGAGGGTATATT | |
| | | (SEQ ID NO: 504) | |
| 60 | PRAME | ACCACTCAAAAACAAACCTTTA | 303 |
| | (SEQ ID NO: 50) | (SEQ ID NO: 505) | |
| | | ATGGGTATGGTGGTTAAGAGTA | |
| | | (SEQ ID NO: 506) | |
| 61 | CDK 4 | TTTTGGTAGTTGGTTATATG | 474 |
| | (SEQ ID NO: 70) | (SEQ ID NO: 508) | |
| | | AAAAATAACACAATAACTCA | |
| | | (SEQ ID NO: 507) | |
| 62 | CDC25A | AGAAGTTGTTTATTGATTGG | 272 |
| | (SEQ ID NO: 72) | (SEQ ID NO: 510) | |
| | | AAAATTAAATCCAAACAAAC | |
| | | (SEQ ID NO: 509) | |
| 63 | CSNK2B | GGGGAAATGGAGAAGTGTAA | 524 |
| | (SEQ ID NO: 67) | (SEQ ID NO: 512) | |
| | | CTACCAATCCCAAAATAACC | |
| | | (SEQ ID NO: 511) | |
| 64 | C-ABL | ATTTATTTTG | 568 |
| | (SEQ ID NO: 62) | (SEQ ID NO: 514) | |
| | | ATCCCAAACTAAAAAAAACAAACCACG | |
| | | (SEQ ID NO: 513) | |
| 65 | L-MYC | AAACCCAAAAACTAAAAAA | 461 |
| | (SEQ ID NO: 61) | (SEQ ID NO: 515) | |
| | | ATGTTATTTAGGTTTGGGTTATTG | |
| | | (SEQ ID NO: 516) | |
| 66 | CSF1 | GAGAATGGGGTGG | |
| | (SEQ ID NO: 65) | (SEQ ID NO: 518) | |
| | | TATAAAAAATCACCCTAACC | |
| | | (SEQ ID NO: 517) | |
| 67 | Me491/TD63 | ACCTAAATCA | |
| | (SEQ ID NO: 68) | (SEQ ID NO: 519) | |
| | | ATGTGAGTTAGGGATTTTTT | |
| | | (SEQ ID NO: 520) | |
| 68 | CD1R3 | ATTATGGTTGGAATTGTAAT | 413 |
| | (SEQ ID NO: 66) | (SEQ ID NO: 522) | |
| | | ACAAAAACAACAAACACCCC | |
| | | (SEQ ID NO: 521) | |
| 69 | N-MYC | GGGAGGAGTATATTTTG | 368 |
| | (SEQ ID NO: 60) | (SEQ ID NO: 524) | |
| | | AAAATATCCTC | |
| | | (SEQ ID NO: 523) | |
| 70 | Tubulin | CTTATATACCCTCCC | 453 |
| | (SEQ ID NO: 64) | (SEQ ID NO: 525) | |
| | | GGGTTTAGAGTTAAGATTG | |
| | | (SEQ ID NO: 526) | |
| 71 | CMYCex3 | AGGAGGAATAAGAAGATGAG | |
| | (SEQ ID NO: 73) | (SEQ ID NO: 528) | |
| | | TTCAATCTCAAAACTCAACC | |
| | | (SEQ ID NO: 527) | |
| 72 | ELK1 | GAAAAAAAAAAACCCAATAT | 523 |
| | (SEQ ID NO: 63) | (SEQ ID NO: 529) | |
| | | ATGGTTTTGTTTAAT | |
| | | (SEQ ID NO: 530) | |
| 73 | AR | GTAGTAGTAGTAGTAAGAGA | 460 |
| | (SEQ ID NO: 69) | (SEQ ID NO: 532) | |
| | | ACCCCCTAAATAATTATCCT | |
| | | (SEQ ID NO: 531) | |
| 74 | Humos | TTTATTGATTGGGAGTAGGT | 494 |
| | (SEQ ID NO: 71) | (SEQ ID NO: 534) | |
| | | CTAATTTTACAAACATCCTA | |
| | | (SEQ ID NO: 533) | |

TABLE 3

Hybridisation oligonucleotides

| | | |
|---|---|---|
| No: | Gene | Oligo: |

| | | |
|---|---|---|
| 1 | MDR1 | TTGGTGGTCGTTTTAAGG |
| | (SEQ ID NO: 37) | (SEQ ID NO: 535) |
| 2 | MDR1 | TTGGTGGTTGTTTTAAGG |
| | (SEQ ID NO: 37) | (SEQ ID NO: 536) |
| 3 | MDR1 | TTGAAAGACGTGTTTATA |
| | (SEQ ID NO: 37) | (SEQ ID NO: 537) |
| 4 | MDR1 | TTGAAAGATGTGTTTATA |
| | (SEQ ID NO: 37) | (SEQ ID NO: 538) |
| 5 | MDR1 | AGGTGTAACGGAAGTTAG |
| | (SEQ ID NO: 37) | (SEQ ID NO: 539) |
| 6 | MDR1 | AGGTGTAATGGAAGTTAG |
| | (SEQ ID NO: 37) | (SEQ ID NO: 540) |
| 7 | MDR1 | TAGTTTTCGAGGAATTA |
| | (SEQ ID NO: 37) | (SEQ ID NO: 541) |
| 8 | MDR1 | TAGTTTTTTGAGGAATTA |
| | (SEQ ID NO: 37) | (SEQ ID NO: 542) |
| 9 | CSNK2B | AGGAGTTTCGGAGGAAAT |
| | (SEQ ID NO: 67) | (SEQ ID NO: 1235) |
| 10 | CSNK2B | AGGAGTTTTGGAGGAAAT |
| | (SEQ ID NO: 67) | (SEQ ID NO: 1236) |
| 11 | CSNK2B | GAGAGTTGCGGAAAGAGA |
| | (SEQ ID NO: 67) | (SEQ ID NO: 543) |
| 12 | CSNK2B | GAGAGTTGTGGAAAGAGA |
| | (SEQ ID NO: 67) | (SEQ ID NO: 544) |
| 13 | CSNK2B | GGGTTTTCGTGATAGT |
| | (SEQ ID NO: 67) | (SEQ ID NO: 545) |
| 14 | CSNK2B | GGGTTTTTGTGATAGT |
| | (SEQ ID NO: 67) | (SEQ ID NO: 546) |
| 15 | CSNK2B | TAGGTTAGCGTATTGGGA |
| | (SEQ ID NO: 67) | (SEQ ID NO: 1117) |
| 16 | CSNK2B | TAGGTTAGTGTATTGGGA |
| | (SEQ ID NO: 67) | (SEQ ID NO: 1118) |
| 17 | EGR4 | GGTGGGAAGCGTATTTAT |
| | (SEQ ID NO: 26) | (SEQ ID NO: 1213) |
| 18 | EGR4 | GGTGGGAAGTGTATTTAT |
| | (SEQ ID NO: 26) | (SEQ ID NO: 1214) |
| 19 | EGR4 | AATAATAACGTTATAGTT |
| | (SEQ ID NO: 26) | (SEQ ID NO: 549) |
| 20 | EGR4 | AATAATAATGTTATAGTT |
| | (SEQ ID NO: 26) | (SEQ ID NO: 550) |
| 21 | EGR4 | TTATAGTTCGAGTTTTTT |
| | (SEQ ID NO: 26) | (SEQ ID NO: 1225) |
| 22 | EGR4 | TTATAGTTTGAGTTTTTT |
| | (SEQ ID NO: 26) | (SEQ ID NO: 1226) |
| 23 | EGR4 | GGAGTTTTCGGTATATAT |
| | (SEQ ID NO: 26) | (SEQ ID NO: 551) |
| 24 | EGR4 | GGAGTTTTTGGTATATAT |
| | (SEQ ID NO: 26) | (SEQ ID NO: 552) |
| 25 | AR | TGTTATTTCGAGAGAGGT |
| | (SEQ ID NO: 69) | (SEQ ID NO: 553) |
| 26 | AR | TGTTATTTTGAGAGAGGT |
| | (SEQ ID NO: 69) | (SEQ ID NO: 554) |
| 27 | AR | AGAGGTTGCGTTTTAGAG |
| | (SEQ ID NO: 69) | (SEQ ID NO: 555) |
| 28 | AR | AGAGGTTGTGTTTTAGAG |
| | (SEQ ID NO: 69) | (SEQ ID NO: 556) |
| 29 | AR | GTAGTATTCGAAGGTAGT |
| | (SEQ ID NO: 69) | (SEQ ID NO: 1255) |
| 30 | AR | GTAGTATTTGAAGGTAGT |
| | (SEQ ID NO: 69) | (SEQ ID NO: 1256) |
| 31 | AR | GGAGGTTTCGGGGGTTTT |
| | (SEQ ID NO: 69) | (SEQ ID NO: 557) |
| 32 | AR | GGAGGTTTTGGGGGTTTT |
| | (SEQ ID NO: 69) | (SEQ ID NO: 558) |
| 33 | CDK4 | GTATGGGGTCGTAGGAAT |
| | (SEQ ID NO: 70) | (SEQ ID NO: 559) |
| 34 | CDK4 | GTATGGGGTTGTAGGAAT |
| | (SEQ ID NO: 70) | (SEQ ID NO: 560) |
| 35 | CDK4 | GGAAGGGTCGTTTAAGGG |
| | (SEQ ID NO: 70) | (SEQ ID NO: 1147) |
| 36 | CDK4 | GGAAGGGTTGTTTAAGGG |
| | (SEQ ID NO: 70) | (SEQ ID NO: 1148) |
| 37 | CDK4 | GGGTTGGCGTGAGGTA |
| | (SEQ ID NO: 70) | (SEQ ID NO: 563) |
| 38 | CDK4 | GGGTTGGTGTGAGGTA |
| | (SEQ ID NO: 70) | (SEQ ID NO: 564) |
| 39 | CDK4 | AGGATTTTCGATGTAAGG |
| | (SEQ ID NO: 70) | (SEQ ID NO: 565) |
| 40 | CDK4 | AGGATTTTTGATGTAAGG |
| | (SEQ ID NO: 70) | (SEQ ID NO: 566) |
| 41 | CDK4 | GGGTTTTACGTGGTTGGA |
| | (SEQ ID NO: 70) | (SEQ ID NO: 567) |
| 42 | CDK4 | GGGTTTTATGTGGTTGGA |
| | (SEQ ID NO: 70) | (SEQ ID NO: 568) |
| 43 | Humos | GAGTTTAACGTAGTAAGG |
| | (SEQ ID NO: 71) | (SEQ ID NO: 1221) |
| 44 | Humos | GAGTTTAATGTAGTAAGG |
| | (SEQ ID NO: 71) | (SEQ ID NO: 1222) |
| 45 | Humos | TATGGAGTTCGGTGGTAA |
| | (SEQ ID NO: 71) | (SEQ ID NO: 569) |
| 46 | Humos | TATGGAGTTTGGTGGTAA |
| | (SEQ ID NO: 71) | (SEQ ID NO: 570) |
| 47 | Humos | TTTATTGTCGTATTGGAG |
| | (SEQ ID NO: 71) | (SEQ ID NO: 571) |
| 48 | Humos | TTTATTGTTGTATTGGAG |
| | (SEQ ID NO: 71) | (SEQ ID NO: 572) |
| 49 | Humos | GTTGTGAACGGTTTTGTTT |
| | (SEQ ID NO: 71) | (SEQ ID NO: 573) |
| 50 | Humos | GTTGTGAATGGTTTGTTT |
| | (SEQ ID NO: 71) | (SEQ ID NO: 574) |
| 51 | RB1 | TTAGATTTCGGGATAGGG |
| | (SEQ ID NO: 52) | (SEQ ID NO: 575) |
| 52 | RB1 | TTAGATTTTGGGATAGGG |
| | (SEQ ID NO: 52) | (SEQ ID NO: 576) |
| 53 | RB1 | TATAGTTTCGTTAAGTGT |
| | (SEQ ID NO: 52) | (SEQ ID NO: 577) |
| 54 | RB1 | TATAGTTTTGTTAAGTGT |
| | (SEQ ID NO: 52) | (SEQ ID NO: 578) |
| 55 | RB1 | GTGTATTTCGGTTTGGAG |
| | (SEQ ID NO: 52) | (SEQ ID NO: 579) |
| 56 | RB1 | GTGTATTTTGGTTTGGAG |
| | (SEQ ID NO: 52) | (SEQ ID NO: 580) |
| 57 | RB1 | TTGGAAGGCGTTTGGATT |
| | (SEQ ID NO: 52) | (SEQ ID NO: 581) |
| 58 | RB1 | TTGGAAGGTGTTTGGATT |
| | (SEQ ID NO: 52) | (SEQ ID NO: 582) |
| 59 | RB1 | TGGATTTACGTTAGGTTT |
| | (SEQ ID NO: 52) | (SEQ ID NO: 583) |
| 60 | RB1 | TGGATTTATGTTAGGTTT |
| | (SEQ ID NO: 52) | (SEQ ID NO: 584) |
| 61 | CDC25A | GTGTAGGTCGGTTTGGTT |
| | (SEQ ID NO: 72) | (SEQ ID NO: 1159) |
| 62 | CDC25A | GTGTAGGTTGGTTTGGTT |
| | (SEQ ID NO: 72) | (SEQ ID NO: 1160) |
| 63 | CDC25A | TTGTTATTCGGAGTTGGG |
| | (SEQ ID NO: 72) | (SEQ ID NO: 1163) |
| 64 | CDC25A | TTGTTATTTGGAGTTGGG |
| | (SEQ ID NO: 72) | (SEQ ID NO: 1164) |
| 65 | CDC25A | GGAGAATAGCGAAGATAG |
| | (SEQ ID NO: 72) | (SEQ ID NO: 589) |
| 66 | CDC25A | GGAGAATAGTGAAGATAG |
| | (SEQ ID NO: 72) | (SEQ ID NO: 590) |
| 67 | CDC25A | GAAAGGTCGGTTTGGT |
| | (SEQ ID NO: 72) | (SEQ ID NO: 591) |
| 68 | CDC25A | GAAAGGTTGGTTTGGT |
| | (SEQ ID NO: 72) | (SEQ ID NO: 592) |
| 69 | GPIb beta | TTTGAGAGCGGGTGGGAG |
| | (SEQ ID NO: 30) | (SEQ ID NO: 593) |
| 70 | GPIb beta | TTTGAGAGTGGGTGGGAG |
| | (SEQ ID NO: 30) | (SEQ ID NO: 594) |
| 71 | GPIb beta | GTGGGAGCGGAAGTTTGA |
| | (SEQ ID NO: 30) | (SEQ ID NO: 595) |
| 72 | GPIb beta | GTGGGAGTGGAAGTTTGA |
| | (SEQ ID NO: 30) | (SEQ ID NO: 596) |
| 73 | GPIb beta | GGTTAGGTCGTAGTATTG |
| | (SEQ ID NO: 30) | (SEQ ID NO: 597) |
| 74 | GPIb beta | GGTTAGGTTGTAGTATTG |
| | (SEQ ID NO: 30) | (SEQ ID NO: 598) |
| 75 | GPIb beta | ATGGGTTTCGGTGAGTTT |
| | (SEQ ID NO: 30) | (SEQ ID NO: 599) |
| 76 | GPIb beta | ATGGGTTTTGGTGAGTTT |
| | (SEQ ID NO: 30) | (SEQ ID NO: 600) |
| 77 | GPIb beta | GGGTTTTCGGTTGTTTTT |
| | (SEQ ID NO: 30) | (SEQ ID NO: 601) |
| 78 | GPIb beta | GGGTTTTCGGTTGTTTTT |
| | (SEQ ID NO: 30) | (SEQ ID NO: 602) |
| 79 | MYOD1 | ATAGTAGTCGGGTGTTGG |
| | (SEQ ID NO: 45) | (SEQ ID NO: 603) |
| 80 | MYOD1 | ATAGTAGTTGGGTGTTGG |
| | (SEQ ID NO: 45) | (SEQ ID NO: 604) |
| 81 | MYOD1 | GTGTTAGTCGTTTAGGGT |
| | (SEQ ID NO: 45) | (SEQ ID NO: 605) |
| 82 | MYOD1 | GTGTTAGTTGTTTAGGGT |
| | (SEQ ID NO: 45) | (SEQ ID NO: 606) |
| 83 | MYOD1 | TAGTTGTTCGTTTGGGTT |
| | (SEQ ID NO: 45) | (SEQ ID NO: 607) |
| 84 | MYOD1 | TAGTTGTTTGTTTGGGTT |
| | (SEQ ID NO: 45) | (SEQ ID NO: 608) |
| 85 | MYOD1 | GGTTATTACGGATAAATA |
| | (SEQ ID NO: 45) | (SEQ ID NO: 609) |
| 86 | MYOD1 | GGTTATTATGGATAAATA |
| | (SEQ ID NO: 45) | (SEQ ID NO: 610) |
| 87 | MYOD1 | AATTAGGTGGGATAGGAG |
| | (SEQ ID NO: 45) | (SEQ ID NO: 611) |
| 88 | MYOD1 | AATTAGGTTGGATAGGAG |
| | (SEQ ID NO: 45) | (SEQ ID NO: 612) |
| 89 | CDH3 | AAATTAGTCGGGTGTGGT |
| | (SEQ ID NO: 17) | (SEQ ID NO: 613) |
| 90 | CDH3 | AAATTAGTTGGGTGTGGT |
| | (SEQ ID NO: 17) | (SEQ ID NO: 614) |
| 91 | CDH3 | TGTGGTGGCGTAAGTTTG |
| | (SEQ ID NO: 17) | (SEQ ID NO: 615) |
| 92 | CDH3 | TGTGGTGGTGTAAGTTTG |
| | (SEQ ID NO: 17) | (SEQ ID NO: 616) |
| 93 | CDH3 | GGAGTTTTCGTTTTTAGT |
| | (SEQ ID NO: 17) | (SEQ ID NO: 617) |
| 94 | CDH3 | GGAGTTTTTGTTTTTAGT |
| | (SEQ ID NO: 17) | (SEQ ID NO: 618) |
| 95 | CDH3 | TAGAATTGCGAGATAGAG |
| | (SEQ ID NO: 17) | (SEQ ID NO: 619) |
| 96 | CDH3 | TAGAATTGTGAGATAGAG |
| | (SEQ ID NO: 17) | (SEQ ID NO: 620) |
| 97 | WT1 | TAGTGAGACGAGGTTTTT |
| | (SEQ ID NO: 59) | (SEQ ID NO: 621) |
| 98 | WT1 | TAGTGAGATGAGGTTTTT |
| | (SEQ ID NO: 59) | (SEQ ID NO: 622) |
| 99 | WT1 | TATATTGGCGAAGGTTAA |
| | (SEQ ID NO: 59) | (SEQ ID NO: 623) |
| 100 | WT1 | TATATTGGTGAAGGTTAA |
| | (SEQ ID NO: 59) | (SEQ ID NO: 624) |
| 101 | WT1 | TGTTATATCGGTTAGTTG |
| | (SEQ ID NO: 59) | (SEQ ID NO: 625) |
| 102 | WT1 | TGTTATATTGGTTAGTTG |
| | (SEQ ID NO: 59) | (SEQ ID NO: 626) |
| 103 | WT1 | TGTTTGGTCGGGTTTGGG |
| | (SEQ ID NO: 59) | (SEQ ID NO: 627) |
| 104 | WT1 | TGTTTGGTTGGGTTTGGG |
| | (SEQ ID NO: 59) | (SEQ ID NO: 628) |
| 105 | WT1 | TTTAGTTTCGATTTTTGG |
| | (SEQ ID NO: 59) | (SEQ ID NO: 629) |
| 106 | WT1 | TTTAGTTTTGATTTTTGG |
| | (SEQ ID NO: 59) | (SEQ ID NO: 630) |
| 107 | MYCL1 | TTGAGGGTCGTTAGGTGG |
| | (SEQ ID NO: 44) | (SEQ ID NO: 631) |
| 108 | MYCL1 | TTGAGGGTTGTTAGGTGG |
| | (SEQ ID NO: 44) | (SEQ ID NO: 632) |
| 109 | MYCL1 | TTTTAGTTCGGAGTGGGT |
| | (SEQ ID NO: 44) | (SEQ ID NO: 633) |
| 110 | MYCL1 | TTTTAGTTTGGAGTGGGT |
| | (SEQ ID NO: 44) | (SEQ ID NO: 634) |
| 111 | MYCL1 | AGTTTAGTCGGTTGGTAT |
| | (SEQ ID NO: 44) | (SEQ ID NO: 635) |
| 112 | MYCL1 | AGTTTAGTTGGTTGGTAT |
| | (SEQ ID NO: 44) | (SEQ ID NO: 636) |
| 113 | MYCL1 | GGGGTTATCGGGGATTTGA |
| | (SEQ ID NO: 44) | (SEQ ID NO: 637) |
| 114 | MYCL1 | GGGGTTATTGGGGATTGA |
| | (SEQ ID NO: 44) | (SEQ ID NO: 638) |
| 115 | ABL1 | GTTTGGGTCGCGAGAGTT |
| | (SEQ ID NO: 2) | (SEQ ID NO: 639) |
| 116 | ABL1 | GTTTGGGTTGTGAGAGTT |
| | (SEQ ID NO: 2) | (SEQ ID NO: 640) |
| 117 | ABL1 | GGATATTGCGGGTGGTTT |
| | (SEQ ID NO: 2) | (SEQ ID NO: 641) |
| 118 | ABL1 | GGATATTGTGGGTGGTTT |
| | (SEQ ID NO: 2) | (SEQ ID NO: 642) |
| 119 | ABL1 | TTTGGTTTCGTTGTGGAG |
| | (SEQ ID NO: 2) | (SEQ ID NO: 643) |
| 120 | ABL1 | TTTGGTTTTGTTGTGGAG |
| | (SEQ ID NO: 2) | (SEQ ID NO: 644) |
| 121 | ABL1 | GGGTTTTGGGGTTGAGGA |
| | (SEQ ID NO: 2) | (SEQ ID NO: 645) |
| 122 | ABL1 | GGGTTTTGTGGTTGAGGA |
| | (SEQ ID NO: 2) | (SEQ ID NO: 646) |
| 123 | ELK1 | TTTGTTTTCGTTGAGTAG |
| | (SEQ ID NO: 27) | (SEQ ID NO: 647) |
| 124 | ELK1 | TTTGTTTTTGTTGAGTAG |
| | (SEQ ID NO: 27) | (SEQ ID NO: 648) |
| 125 | ELK1 | TTTATTTTCGTTTTTGGG |
| | (SEQ ID NO: 27) | (SEQ ID NO: 649) |
| 126 | ELK1 | TTTATTTTTGTTTTTGGG |
| | (SEQ ID NO: 27) | (SEQ ID NO: 650) |
| 127 | ELK1 | GAAGGGTTGGTTTTTTAA |
| | (SEQ ID NO: 27) | (SEQ ID NO: 651) |
| 128 | ELK1 | GAAGGGTTTGTTTTTTAA |
| | (SEQ ID NO: 27) | (SEQ ID NO: 652) |
| 129 | APC | TATTAGAGGGTTTTAAAG |
| | (SEQ ID NO: 4) | (SEQ ID NO: 653) |
| 130 | APC | TATTAGAGTGTTTTAAAG |
| | (SEQ ID NO: 4) | (SEQ ID NO: 654) |
| 131 | APC | GTTTTTTTCGATTTGGGT |
| | (SEQ ID NO: 4) | (SEQ ID NO: 655) |
| 132 | APC | GTTTTTTTTGATTTGGGT |
| | (SEQ ID NO: 4) | (SEQ ID NO: 656) |
| 133 | ARHI | TGTTGTTGCGTAGTAGAA |
| | (SEQ ID NO: 6) | (SEQ ID NO: 657) |
| 134 | ARHI | TGTTGTTGTGTAGTAGAA |
| | (SEQ ID NO: 6) | (SEQ ID NO: 658) |
| 135 | ARHI | GAATTATTCGTAGTTTTG |
| | (SEQ ID NO: 6) | (SEQ ID NO: 659) |
| 136 | ARHI | GAATTATTTGTAGTTTTG |
| | (SEQ ID NO: 6) | (SEQ ID NO: 660) |
| 137 | ARHI | GATAGTTTCGTTGGGAAG |
| | (SEQ ID NO: 6) | (SEQ ID NO: 661) |
| 138 | ARHI | GATAGTTTTGTTGGGAAG |
| | (SEQ ID NO: 6) | (SEQ ID NO: 662) |
| 139 | ARHI | TAGAAGAACGAGGTTTGA |
| | (SEQ ID NO: 6) | (SEQ ID NO: 663) |
| 140 | ARHI | TAGAAGAATGAGGTTTGA |
| | (SEQ ID NO: 6) | (SEQ ID NO: 664) |
| 141 | ARHI | TATAGTTGCGTAGGTAGT |
| | (SEQ ID NO: 6) | (SEQ ID NO: 665) |
| 142 | ARHI | TATAGTTGTGTAGGTAGT |
| | (SEQ ID NO: 6) | (SEQ ID NO: 666) |
| 143 | BCL2 | AGAGGTGTCGTTGGTTTT |
| | (SEQ ID NO: 9) | (SEQ ID NO: 667) |
| 144 | BCL2 | AGAGGTGTTGTTGGTTTT |
| | (SEQ ID NO: 9) | (SEQ ID NO: 668) |
| 145 | BCL2 | TAAGTTGTCGTAGAGGGG |
| | (SEQ ID NO: 9) | (SEQ ID NO: 669) |
| 146 | BCL2 | TAAGTTGTTGTAGAGGGG |
| | (SEQ ID NO: 9) | (SEQ ID NO: 670) |
| 147 | BCL2 | AGGGGTTACGAGTGGGAT |
| | (SEQ ID NO: 9) | (SEQ ID NO: 671) |
| 148 | BCL2 | AGGGGTTATGAGTGGGAT |
| | (SEQ ID NO: 9) | (SEQ ID NO: 672) |
| 149 | BCL2 | TTTGTTACGGTGGTGGA |
| | (SEQ ID NO: 9) | (SEQ ID NO: 673) |
| 150 | BCL2 | TTTTGTTATGGTGGTGGA |
| | (SEQ ID NO: 9) | (SEQ ID NO: 674) |
| 151 | CCND2 | AGTTGGGTCGGGTTAGTT |
| | (SEQ ID NO: 13) | (SEQ ID NO: 675) |
| 152 | CCND2 | AGTTGGGTTGGGTTAGTT |
| | (SEQ ID NO: 13) | (SEQ ID NO: 676) |
| 153 | CCND2 | TTTAATAACGAGAGGGGA |
| | (SEQ ID NO: 13) | (SEQ ID NO: 677) |
| 154 | CCND2 | TTTAATAATGAGAGGGGA |
| | (SEQ ID NO: 13) | (SEQ ID NO: 678) |
| 155 | CCND2 | TTAGTTTGCGTTATCGTT |
| | (SEQ ID NO: 13) | (SEQ ID NO: 679) |
| 156 | CCND2 | TTAGTTTGTGTTATTGTT |
| | (SEQ ID NO: 13) | (SEQ ID NO: 680) |
| 157 | CCND2 | TTTTAGAGCGGAGAAGAG |
| | (SEQ ID NO: 13) | (SEQ ID NO: 681) |
| 158 | CCND2 | TTTTAGAGTGGAGAAGAG |
| | (SEQ ID NO: 13) | (SEQ ID NO: 682) |
| 159 | CCND2 | GGTAGTTTCGAGGTTTTG |
| | (SEQ ID NO: 13) | (SEQ ID NO: 683) |
| 160 | CCND2 | GGTAGTTTTGAGGTTTTG |
| | (SEQ ID NO: 13) | (SEQ ID NO: 684) |
| 161 | CDH1 | AGTTTCGACGTTATTGAG |
| | (SEQ ID NO: 16) | (SEQ ID NO: 685) |
| 162 | CDH1 | AGTTTTGATGTTATTGAG |
| | (SEQ ID NO: 16) | (SEQ ID NO: 686) |
| 163 | CDH1 | AGAGGTTGCGGTTTTAAG |
| | (SEQ ID NO: 16) | (SEQ ID NO: 687) |
| 164 | CDH1 | AGAGGTTGTGGTTTTAAG |
| | (SEQ ID NO: 16) | (SEQ ID NO: 688) |
| 165 | CDH1 | AGTAGCGTCGAGAGGTTG |
| | (SEQ ID NO: 16) | (SEQ ID NO: 689) |
| 166 | CDH1 | AGTAGTGTTGAGAGGTTG |
| | (SEQ ID NO: 16) | (SEQ ID NO: 690) |
| 167 | CDH1 | AGGGGATTCGGGGTATTT |
| | (SEQ ID NO: 16) | (SEQ ID NO: 691) |
| 168 | CDH1 | AGGGGATTTGGGGTATTT |
| | (SEQ ID NO: 16) | (SEQ ID NO: 692) |
| 169 | CDKN1A | AGGTGTATCGTTTTTATA |
| | (SEQ ID NO: 19) | (SEQ ID NO: 693) |
| 170 | CDKN1A | AGGTGTATTGTTTTTATA |
| | (SEQ ID NO: 19) | (SEQ ID NO: 694) |
| 171 | CDKN1A | TGGGTTAGCGGTGAGTTA |
| | (SEQ ID NO: 19) | (SEQ ID NO: 695) |
| 172 | CDKN1A | TGGGTTAGTGGTGAGTTA |
| | (SEQ ID NO: 19) | (SEQ ID NO: 696) |
| 173 | CDKN1A | GTTTATTTCGTGGGGAAA |
| | (SEQ ID NO: 19) | (SEQ ID NO: 697) |
| 174 | CDKN1A | GTTTATTTTGTGGGGAAA |
| | (SEQ ID NO: 19) | (SEQ ID NO: 698) |
| 175 | CDKN1A | TTGGAATTCGGTTAGGTT |
| | (SEQ ID NO: 19) | (SEQ ID NO: 699) |
| 176 | CDKN1A | TTGGAATTTGGTTAGGTT |
| | (SEQ ID NO: 19) | (SEQ ID NO: 700) |
| 177 | CDKN1B | AAGAGAAACGTTGGAATA |
| | (p27 Kip1) | (SEQ ID NO: 701) |
| | (SEQ ID NO: 20) | |
| 178 | CDKN1B | AAGAGAAATGTTGGAATA |
| | (p27 Kip1) | (SEQ ID NO: 702) |
| | (SEQ ID NO: 20) | |
| 179 | CDKN1B | TTTGATTTCGAGGGGAGT |
| | (p27 Kip1) | (SEQ ID NO: 703) |
| | (SEQ ID NO: 20) | |
| 180 | CDKN1B | TTTGATTTTGAGGGGAGT |
| | (p27 Kip1) | (SEQ ID NO: 704) |
| | (SEQ ID NO: 20) | |
| 181 | CDKN1B | GTATTTGGCGGTTGGATT |
| | (p27 Kip1) | (SEQ ID NO: 705) |
| | (SEQ ID NO: 20) | |
| 182 | CDKN1B | GTATTTGGTGGTTGGATT |
| | (p27 Kip1) | (SEQ ID NO: 706) |
| | (SEQ ID NO: 20) | |
| 183 | CDKN1B | TATAATTTCGGGAAAGAA |
| | (p27 Kip1) | (SEQ ID NO: 707) |
| | (SEQ ID NO: 20) | |
| 184 | CDKN1B | TATAATTTTGGGAAAGAA |
| | (p27 Kip1) | (SEQ ID NO: 708) |
| | (SEQ ID NO: 20) | |
| 185 | CDKN2a | AGAGTGAACGTATTTAAA |
| | (SEQ ID NO: 22) | (SEQ ID NO: 709) |
| 186 | CDKN2a | AGAGTGAATGTATTTAAA |
| | (SEQ ID NO: 22) | (SEQ ID NO: 710) |
| 187 | CDKN2a | GTTATATTCGTTAAGTGT |
| | (SEQ ID NO: 22) | (SEQ ID NO: 711) |
| 188 | CDKN2a | GTTATATTTGTTAAGTGT |
| | (SEQ ID NO: 22) | (SEQ ID NO: 712) |
| 189 | CDKN2a | TAAGTGTTCGGAGTTAAT |
| | (SEQ ID NO: 22) | (SEQ ID NO: 713) |
| 190 | CDKN2a | TAAGTGTTTGGAGTTAAT |
| | (SEQ ID NO: 22) | (SEQ ID NO: 714) |
| 191 | CDKN2a | GTTAGTATCGGAGGAAGA |
| | (SEQ ID NO: 22) | (SEQ ID NO: 715) |
| 192 | CDKN2a | GTTAGTATTGGAGGAAGA |
| | (SEQ ID NO: 22) | (SEQ ID NO: 716) |
| 193 | CDKN2a | GGAGTTTTCGGTTGATTG |
| | (SEQ ID NO: 22) | (SEQ ID NO: 717) |
| 194 | CDKN2a | GGAGTTTTTGGTTGATTG |
| | (SEQ ID NO: 22) | (SEQ ID NO: 718) |
| 195 | CDKN2a | TTGTTTAACGTATCGAAT |
| | (SEQ ID NO: 22) | (SEQ ID NO: 719) |
| 196 | CDKN2a | TTGTTTAATGTATTGAAT |
| | (SEQ ID NO: 22) | (SEQ ID NO: 720) |
| 197 | CDKN2a | AATAGTTACGGTCGGAGG |
| | (SEQ ID NO: 22) | (SEQ ID NO: 721) |
| 198 | CDKN2a | AATAGTTATGGTTGGAGG |
| | (SEQ ID NO: 22) | (SEQ ID NO: 722) |
| 199 | CDKN2B | ATATTTAGCGAGTAGTGT |
| | (SEQ ID NO: 23) | (SEQ ID NO: 723) |
| 200 | CDKN2B | ATATTTAGTGAGTAGTGT |
| | (SEQ ID NO: 23) | (SEQ ID NO: 724) |
| 201 | CDKN2B | ATAGGGGGCGGAGTTTAA |
| | (SEQ ID NO: 23) | (SEQ ID NO: 725) |
| 202 | CDKN2B | ATAGGGGGTGGAGTTTAA |
| | (SEQ ID NO: 23) | (SEQ ID NO: 726) |
| 203 | CDKN2B | TTATTGTACGGGGTTTTA |
| | (SEQ ID NO: 23) | (SEQ ID NO: 727) |
| 204 | CDKN2B | TTATTGTATGGGGTTTTA |
| | (SEQ ID NO: 23) | (SEQ ID NO: 728) |
| 205 | CDKN2B | TTTTAAGTCGTAGAAGGA |
| | (SEQ ID NO: 23) | (SEQ ID NO: 729) |
| 206 | CDKN2B | TTTTAAGTTGTAGAAGGA |
| | (SEQ ID NO: 23) | (SEQ ID NO: 730) |
| 207 | DAPK1 | GTTGGAGTCGAGGTTTGA |
| | (SEQ ID NO: 25) | (SEQ ID NO: 731) |
| 208 | DAPK1 | GTTGGAGTTGAGGTTTGA |
| | (SEQ ID NO: 25) | (SEQ ID NO: 732) |
| 209 | DAPK1 | GAAGGGAGCGTATTTTAT |
| | (SEQ ID NO: 25) | (SEQ ID NO: 733) |
| 210 | DAPK1 | GAAGGGAGTGTATTTTAT |
| | (SEQ ID NO: 25) | (SEQ ID NO: 734) |
| 211 | DAPK1 | TTGTTTTTCGGAAATTTG |
| | (SEQ ID NO: 25) | (SEQ ID NO: 735) |
| 212 | DAPK1 | TTGTTTTTTGGAAATTTG |
| | (SEQ ID NO: 25) | (SEQ ID NO: 736) |
| 213 | FOS | AATGTTTTCGTACGTAGG |
| | (SEQ ID NO: 29) | (SEQ ID NO: 737) |
| 214 | FOS | AATGTTTTTGTATGTAGG |
| | (SEQ ID NO: 29) | (SEQ ID NO: 738) |
| 215 | FOS | TATATGGTCGAGAAAAAT |
| | (SEQ ID NO: 29) | (SEQ ID NO: 739) |
| 216 | FOS | TATATGGTTGAGAAAAAT |
| | (SEQ ID NO: 29) | (SEQ ID NO: 740) |
| 217 | FOS | TTTAGTATCGTAAAGTAG |
| | (SEQ ID NO: 29) | (SEQ ID NO: 741) |
| 218 | FOS | TTTAGTATTGTAAAGTAG |
| | (SEQ ID NO: 29) | (SEQ ID NO: 742) |
| 219 | FOS | GTATTGTTCGAGTTCGAG |
| | (SEQ ID NO: 29) | (SEQ ID NO: 743) |
| 220 | FOS | GTATTGTTTGAGTTTGAG |
| | (SEQ ID NO: 29) | (SEQ ID NO: 744) |
| 221 | GSTP1 | GGTTTTTTCGGTTAGTTG |
| | (SEQ ID NO: 33) | (SEQ ID NO: 745) |
| 222 | GSTP1 | GGTTTTTTCGGTTAGTTG |
| | (SEQ ID NO: 33) | (SEQ ID NO: 746) |
| 223 | GSTP1 | TTTTAGGGCGTTTTTTTG |
| | (SEQ ID NO: 33) | (SEQ ID NO: 747) |
| 224 | GSTP1 | TTTTAGGGTGTTTTTTTG |
| | (SEQ ID NO: 33) | (SEQ ID NO: 748) |
| 225 | GSTP1 | GTAGTTTTCGTTATTAGT |
| | (SEQ ID NO: 33) | (SEQ ID NO: 749) |
| 226 | GSTP1 | GTAGTTTTTGTTATTAGT |
| | (SEQ ID NO: 33) | (SEQ ID NO: 750) |
| 227 | HIC-1 | TTAAAAGGGGGTATAGGG |
| | (SEQ ID NO: 34) | (SEQ ID NO: 751) |
| 228 | HIC-1 | TTAAAATGGTGTATAGGG |
| | (SEQ ID NO: 34) | (SEQ ID NO: 752) |
| 229 | HIC-1 | AGGAGATTGGAAAGTTTA |
| | (SEQ ID NO: 34) | (SEQ ID NO: 753) |
| 230 | HIC-1 | AGGAGATTTGAAAGTTTA |
| | (SEQ ID NO: 34) | (SEQ ID NO: 754) |
| 231 | HIC-1 | GGGTTTTTACGTGGTTGTT |
| | (SEQ ID NO: 34) | (SEQ ID NO: 755) |
| 232 | HIC-1 | GGGTTTTATGTGGTTGTT |
| | (SEQ ID NO: 34) | (SEQ ID NO: 756) |
| 233 | HIC-1 | TTTTAGAGCGTTAGGGTT |
| | (SEQ ID NO: 34) | (SEQ ID NO: 757) |
| 234 | HIC-1 | TTTTAGAGTGTTAGGGTT |
| | (SEQ ID NO: 34) | (SEQ ID NO: 758) |
| 235 | IGF2 | AGTTTGAACGATGTAAGA |
| | (SEQ ID NO: 36) | (SEQ ID NO: 759) |
| 236 | IGF2 | AGTTTGAATGATGTAAGA |
| | (SEQ ID NO: 36) | (SEQ TD NO: 760) |
| 237 | IGF2 | GGTTATTAGGATAATTTG |
| | (SEQ ID NO: 36) | (SEQ ID NO: 761) |
| 238 | IGF2 | GGTTATTATGATAATTTG |
| | (SEQ ID NO: 36) | (SEQ ID NO: 762) |
| 239 | IGF2 | TTGTATGGTGGAGTTTAT |
| | (SEQ ID NO: 36) | (SEQ ID NO: 763) |
| 240 | IGF2 | TTGTATGGTTGAGTTTAT |
| | (SEQ ID NO: 36) | (SEQ ID NO: 764) |
| 241 | IGF2 | GATTAGGGCGGGAAATAT |
| | (SEQ ID NO: 36) | (SEQ ID NO: 765) |
| 242 | IGF2 | GATTAGGGTGGGAAATAT |
| | (SEQ ID NO: 36) | (SEQ ID NO: 766) |
| 243 | IGF2 | TGGAGTTTAGGGAGGTTT |
| | (SEQ ID NO: 36) | (SEQ ID NO: 767) |
| 244 | IGF2 | TGGAGTTTATGGAGGTTT |
| | (SEQ ID NO: 36) | (SEQ ID NO: 768) |
| 245 | MGMT | TAAGGATACGAGTTATAT |
| | (SEQ ID NO: 38) | (SEQ ID NO: 769) |
| 246 | MGMT | TAAGGATATGAGTTATAT |
| | (SEQ ID NO: 38) | (SEQ ID NO: 770) |
| 247 | MGMT | TTGGAGAGCGGTTGAGTT |
| | (SEQ ID NO: 38) | (SEQ ID NO: 771) |
| 248 | MGMT | TTGGAGAGTGGTTGAGTT |
| | (SEQ ID NO: 38) | (SEQ ID NO: 772) |
| 249 | MGMT | TAGGTTATCGGTGATTGT |
| | (SEQ ID NO: 38) | (SEQ ID NO: 773) |
| 250 | MGMT | TAGGTTATTGGTGATTGT |
| | (SEQ ID NO: 38) | (SEQ ID NO: 774) |
| 251 | MGMT | TAGGGGAGCGGTTTTAGG |
| | (SEQ ID NO: 38) | (SEQ ID NO: 775) |
| 252 | MGMT | TAGGGGAGTGGTTTTAGG |
| | (SEQ ID NO: 38) | (SEQ ID NO: 776) |
| 253 | MGMT | AGTAGGATCGGGATTTTT |
| | (SEQ ID NO: 38) | (SEQ ID NO: 777) |
| 254 | MGMT | AGTAGGATTGGGATTTTT |
| | (SEQ ID NO: 38) | (SEQ ID NO: 778) |
| 255 | MLH1 | TTGAGAAGCGTTAAGTAT |
| | (SEQ ID NO: 39) | (SEQ ID NO: 779) |
| 256 | MLH1 | TTGAGAAGTGTTAAGTAT |
| | (SEQ ID NO: 39) | (SEQ ID NO: 780) |
| 257 | MLH1 | TTAGGTAGCGGGTAGTAG |
| | (SEQ ID NO: 39) | (SEQ ID NO: 781) |
| 258 | MLH1 | TTAGGTAGTGGGTAGTAG |
| | (SEQ ID NO: 39) | (SEQ ID NO: 782) |
| 259 | MLH1 | GTAGTAGTCGTTTTAGGG |
| | (SEQ ID NO: 39) | (SEQ ID NO: 783) |
| 260 | MLH1 | GTAGTAGTTGTTTTAGGG |
| | (SEQ ID NO: 39) | (SEQ ID NO: 784) |
| 261 | MLH1 | ATAGTTGTCGTTGAAGGG |
| | (SEQ ID NO: 39) | (SEQ ID NO: 785) |
| 262 | MLH1 | ATAGTTGTTGTTGAAGGG |
| | (SEQ ID NO: 39) | (SEQ ID NO: 786) |
| 263 | MLH1 | TTGGATGGCGTAAGTTAT |
| | (SEQ ID NO: 39) | (SEQ ID NO: 787) |
| 264 | MLH1 | TTGGATGGTGTAAGTTAT |
| | (SEQ ID NO: 39) | (SEQ ID NO: 788) |
| 265 | MOS | AGTAGTTTCGTAGGTAGT |
| | (SEQ ID NO: 40) | (SEQ ID NO: 789) |
| 266 | MOS | AGTAGTTTTGTAGGTAGT |
| | (SEQ ID NO: 40) | (SEQ ID NO: 790) |
| 267 | MOS | GTAAGTCGTTTTGTATAT |
| | (SEQ ID NO: 40) | (SEQ ID NO: 791) |
| 268 | MOS | GTAAGTTGTTTTGTATAT |
| | (SEQ ID NO: 40) | (SEQ ID NO: 792) |
| 269 | MOS | ATGTTAGTCGGTTTTTGG |
| | (SEQ ID NO: 40) | (SEQ ID NO: 793) |
| 270 | MOS | ATGTTAGTTGGTTTTTGG |
| | (SEQ ID NO: 40) | (SEQ ID NO: 794) |
| 271 | MPL | GTGGTGGGCGTTTGTAAT |
| | (SEQ ID NO: 42) | (SEQ ID NO: 795) |
| 272 | MPL | GTGGTGGGTGTTTGTAAT |
| | (SEQ ID NO: 42) | (SEQ ID NO: 796) |
| 273 | MPL | TTTGAGGTCGGGAGTTTA |
| | (SEQ ID NO: 42) | (SEQ ID NO: 797) |
| 274 | MPL | TTTGAGGTTGGGAGTTTA |
| | (SEQ ID NO: 42) | (SEQ ID NO: 798) |
| 275 | MPL | TGTAGTGAGTCGAGATTA |
| | (SEQ ID NO: 42) | (SEQ ID NO: 1227) |
| 276 | MPL | TGTAGTGAGTTGAGATTA |
| | (SEQ ID NO: 42) | (SEQ ID NO: 1228) |
| 277 | MPL | GGTAATAGAGCGAGATTT |
| | (SEQ ID NO: 42) | (SEQ ID NO: 799) |
| 278 | MPL | GGTAATAGAGTGAGATTT |
| | (SEQ ID NO: 42) | (SEQ ID NO: 800) |
| 279 | MPL | TTAAGTAGCGGGTAAGGT |
| | (SEQ ID NO: 42) | (SEQ ID NO: 801) |
| 280 | MPL | TTAAGTAGTGGGTAAGGT |
| | (SEQ ID NO: 42) | (SEQ ID NO: 802) |
| 281 | MYC | TTAGAGTGTTCGGTTGTT |
| | (SEQ ID NO: 43) | (SEQ ID NO: 803) |
| 282 | MYC | TTAGAGTGTTTGGTTGTT |
| | (SEQ ID NO: 43) | (SEQ ID NO: 804) |
| 283 | MYC | TTATAATGCGAGGGTTTG |
| | (SEQ ID NO: 43) | (SEQ ID NO: 805) |
| 284 | MYC | TTATAATGTGAGGGTTTG |
| | (SEQ ID NO: 43) | (SEQ ID NO: 806) |
| 285 | MYC | AGGATTTTCGAGTTGTGT |
| | (SEQ ID NO: 43) | (SEQ ID NO: 807) |
| 286 | MYC | AGGATTTTTGAGTTGTGT |
| | (SEQ ID NO: 43) | (SEQ ID NO: 808) |
| 287 | MYC | AATTTTAGCGAGAGGTAG |
| | (SEQ ID NO: 43) | (SEQ ID NO: 809) |
| 288 | MYC | AATTTTAGTGAGAGGTAG |
| | (SEQ ID NO: 43) | (SEQ ID NO: 810) |
| 289 | MYC | TTGTGGGCGTTTTGGGAA |
| | (SEQ ID NO: 43) | (SEQ ID NO: 811) |
| 290 | MYC | TTGTGGGTGTTTTGGGAA |
| | (SEQ ID NO: 43) | (SEQ ID NO: 812) |
| 291 | N33 | TTGGTTCGGGAAAGGTAA |
| | (SEQ ID NO: 46) | (SEQ ID NO: 1211) |
| 292 | N33 | TTGGTTTGGGAAAGGTAA |
| | (SEQ ID NO: 46) | (SEQ ID NO: 1212) |
| 293 | N33 | TGTTATTTCGGAGGGTTT |
| | (SEQ ID NO: 46) | (SEQ ID NO: 1217) |
| 294 | N33 | TGTTATTTTGGAGGGTTT |
| | (SEQ ID NO: 46) | (SEQ ID NO: 1218) |
| 295 | N33 | GTTTAGTTAGCGGGTTTT |
| | (SEQ ID NO: 46) | (SEQ ID NO: 1219) |
| 296 | N33 | GTTTAGTTAGTGGGTTTT |
| | (SEQ ID NO: 46) | (SEQ ID NO: 1220) |
| 297 | N33 | ATTTAGTTCGGGGGAGGA |
| | (SEQ ID NO: 46) | (SEQ ID NO: 1215) |
| 298 | N33 | ATTTAGTTTGGGGGAGGA |
| | (SEQ ID NO: 46) | (SEQ ID NO: 1216) |
| 299 | PMS2 | AGATTATACGTTGTATGT |
| | (SEQ ID NO: 49) | (SEQ ID NO: 813) |
| 300 | PMS2 | AGATTATATGTTGTATGT |
| | (SEQ ID NO: 49) | (SEQ ID NO: 814) |
| 301 | PMS2 | TAGGAGTTCGAGATTAGT |
| | (SEQ ID NO: 49) | (SEQ ID NO: 815) |
| 302 | PMS2 | TAGGAGTTTGAGATTAGT |
| | (SEQ ID NO: 49) | (SEQ ID NO: 816) |
| 303 | PMS2 | AAATTAGTCGGGTTTAGT |
| | (SEQ ID NO: 49) | (SEQ ID NO: 817) |
| 304 | PMS2 | AAATTAGTTGGGTTTAGT |
| | (SEQ ID NO: 49) | (SEQ ID NO: 818) |
| 305 | PMS2 | GTGGATAGCGTTTGTAAT |
| | (SEQ ID NO: 49) | (SEQ ID NO: 819) |
| 306 | PMS2 | GTGGATAGTGTTTGTAAT |
| | (SEQ ID NO: 49) | (SEQ ID NO: 820) |
| 307 | PTEN | GGATTTTGCGTTCGTATT |
| | (SEQ ID NO: 51) | (SEQ ID NO: 821) |
| 308 | PTEN | GGATTTTGTGTTTGTATT |
| | (SEQ ID NO: 51) | (SEQ ID NO: 822) |
| 309 | PTEN | AGAGTTATCGTTTTGTTT |
| | (SEQ ID NO: 51) | (SEQ ID NO: 823) |
| 310 | PTEN | AGAGTTATTGTTTTGTTT |
| | (SEQ ID NO: 51) | (SEQ ID NO: 824) |
| 311 | PTEN | TGATGTGGCGGGATTTTT |
| | (SEQ ID NO: 51) | (SEQ ID NO: 825) |
| 312 | PTEN | TGATGTGGTGGGATTTTT |
| | (SEQ ID NO: 51) | (SEQ ID NO: 826) |
| 313 | RBL2 | ATTAGTGTCGTTGTTAAG |
| | (SEQ ID NO: 53) | (SEQ ID NO: 827) |
| 314 | RBL2 | ATTAGTGTTGTTGTTAAG |
| | (SEQ ID NO: 53) | (SEQ ID NO: 828) |
| 315 | RBL2 | AGATTATACGGATAAGGG |
| | (SEQ ID NO: 53) | (SEQ ID NO: 829) |
| 316 | RBL2 | AGATTATATGGATAAGGG |
| | (SEQ ID NO: 53) | (SEQ ID NO: 830) |
| 317 | SFN | ATAGAGTTCGGTATTGGT |
| | (SEQ ID NO: 55) | (SEQ ID NO: 831) |
| 318 | SFN | ATAGAGTTTGGTATTGGT |
| | (SEQ ID NO: 55) | (SEQ ID NO: 832) |
| 319 | SFN | GAGTAGGTCGAACGTTAT |
| | (SEQ ID NO: 55) | (SEQ ID NO: 833) |
| 320 | SFN | GAGTAGGTTGAATGTTAT |
| | (SEQ ID NO: 55) | (SEQ ID NO: 834) |
| 321 | SFN | AAAAGTAACGAGGAGGGT |
| | (SEQ ID NO: 55) | (SEQ ID NO: 835) |
| 322 | SFN | AAAAGTAATGAGGAGGGT |
| | (SEQ ID NO: 55) | (SEQ ID NO: 836) |
| 323 | TGFBR2 | ATTTGGAGCGAGGAATTT |
| | (SEQ ID NO: 57) | (SEQ ID NO: 837) |
| 324 | TGFBR2 | ATTTGGAGTGAGGAATTT |
| | (SEQ ID NO: 57) | (SEQ ID NO: 838) |
| 325 | TGFBR2 | TTGAAAGTCGGTTAAAGT |
| | (SEQ ID NO: 57) | (SEQ ID NO: 839) |
| 326 | TGFBR2 | TTGAAAGTTGGTTAAAGT |
| | (SEQ ID NO: 57) | (SEQ ID NO: 840) |
| 327 | TGFBR2 | AAAGTTTTCGGAGGGGTT |
| | (SEQ ID NO: 57) | (SEQ ID NO: 841) |
| 328 | TGFBR2 | AAAGTTTTTGGAGGGGTT |
| | (SEQ ID NO: 57) | (SEQ ID NO: 842) |
| 329 | TGFBR2 | GGTAGTTACGAGAGAGTT |
| | (SEQ ID NO: 57) | (SEQ ID NO: 843) |
| 330 | TGFBR2 | GGTAGTTATGAGAGAGTT |
| | (SEQ ID NO: 57) | (SEQ ID NO: 844) |
| 331 | TP73 | TGATTTAGCGTAGGTTTG |
| | (SEQ ID NO: 58) | (SEQ ID NO: 845) |
| 332 | TP73 | TGATTTAGTGTAGGTTTG |
| | (SEQ ID NO: 58) | (SEQ ID NO: 846) |
| 333 | TP73 | TTTGGTGCGCGTAGAGAA |
| | (SEQ ID NO: 58) | (SEQ ID NO: 1229) |
| 334 | TP73 | TTTGGTGTGTGTAGAGAA |
| | (SEQ ID NO: 58) | (SEQ ID NO: 1230) |
| 335 | TP73 | TTAGAGTTCGAGTTTATA |
| | (SEQ ID NO: 58) | (SEQ ID NO: 847) |
| 336 | TP73 | TTAGAGTTTGAGTTTATA |
| | (SEQ ID NO: 58) | (SEQ ID NO: 848) |
| 337 | TP73 | GAGTGTTCGCGTTTTGGG |
| | (SEQ ID NO: 58) | (SEQ ID NO: 849) |
| 338 | TP73 | GAGTGTTTGTGTTTTGGG |
| | (SEQ ID NO: 58) | (SEQ ID NO: 850) |
| 339 | TP73 | AAGATGTGCGAGTTAGTC |
| | (SEQ ID NO: 58) | (SEQ ID NO: 851) |
| 340 | TP73 | AAGATGTGTGAGTTAGTC |
| | (SEQ ID NO: 58) | (SEQ ID NO: 852) |
| 341 | TP73 | AAGTTACGGGTTTTATTG |
| | (SEQ ID NO: 58) | (SEQ ID NO: 853) |
| 342 | TP73 | AAGTTATGGGTTTTATTG |
| | (SEQ ID NO: 58) | (SEQ ID NO: 854) |
| 343 | TP73 | TTAGATTACGGGTTTTAT |
| | (SEQ ID NO: 58) | (SEQ ID NO: 855) |
| 344 | TP73 | TTAGATTATGGGTTTTAT |
| | (SEQ ID NO: 58) | (SEQ ID NO: 856) |
| 345 | TP73 | TAAGTAGCGTCGTTATTG |
| | (SEQ ID NO: 58) | (SEQ ID NO: 857) |
| 346 | TP73 | TAAGTAGTGTTGTTATTG |
| | (SEQ ID NO: 58) | (SEQ ID NO: 858) |
| 347 | TP73 | GGAAGTTTCGATGGTTTA |
| | (SEQ ID NO: 58) | (SEQ ID NO: 859) |
| 348 | TP73 | GGAAGTTTTGATGGTTTA |
| | (SEQ ID NO: 58) | (SEQ ID NO: 860) |
| 349 | CDKN1C | ATGAAGAACGGTTAAGGG |
| | (SEQ ID NO: 21) | (SEQ ID NO: 861) |
| 350 | CDKN1C | ATGAAGAATGGTTAAGGG |
| | (SEQ ID NO: 21) | (SEQ ID NO: 862) |
| 351 | CDKN1C | TTAAGTTACGGTTATTAG |
| | (SEQ ID NO: 21) | (SEQ ID NO: 863) |
| 352 | CDKN1C | TTAAGTTATGGTTATTAG |
| | (SEQ ID NO: 21) | (SEQ ID NO: 864) |
| 353 | CDKN1C | TTAGTGTTCGTTTGGAAT |
| | (SEQ ID NO: 21) | (SEQ ID NO: 865) |
| 354 | CDKN1C | TTAGTGTTTGTTTGGAAT |
| | (SEQ ID NO: 21) | (SEQ ID NO: 866) |
| 355 | CDKN1C | AGGAGTTGCGGTGGGAAT |
| | (SEQ ID NO: 21) | (SEQ ID NO: 867) |
| 356 | CDKN1C | AGGAGTTGTGGTGGGAAT |
| | (SEQ ID NO: 21) | (SEQ ID NO: 868) |
| 357 | GSK3β | GGGTAAAGCGCGGATATT |
| | (SEQ ID NO: 32) | (SEQ ID NO: 869) |
| 358 | GSK3β | GGGTAAAGTGTGGATATT |
| | (SEQ ID NO: 32) | (SEQ ID NO: 870) |
| 359 | GSK3β | TATGTTTTCGGCGAATGG |
| | (SEQ ID NO: 32) | (SEQ ID NO: 871) |
| 360 | GSK3β | TATGTTTTTGGTGAATGG |
| | (SEQ ID NO: 32) | (SEQ ID NO: 872) |
| 361 | GSK3β | GGGGAATAGTCGAGGAGT |
| | (SEQ ID NO: 32) | (SEQ ID NO: 873) |
| 362 | GSK3β | GGGGAATAGTTGAGGAGT |
| | (SEQ ID NO: 32) | (SEQ ID NO: 874) |
| 363 | GSK3β | AGGAGTCGTTGTTTGGGG |
| | (SEQ ID NO: 32) | (SEQ ID NO: 875) |
| 364 | GSK3β | AGGAGTTGTTGTTTGGGG |
| | (SEQ ID NO: 32) | (SEQ ID NO: 876) |
| 365 | CDC2 | TAGTTATTCGGGAAGGTA |
| | (SEQ ID NO: 14) | (SEQ ID NO: 877) |
| 366 | CDC2 | TAGTTATTGGGAAGGTA |
| | (SEQ ID NO: 14) | (SEQ ID NO: 878) |
| 367 | CDC2 | AGGTATTGCGGTAGTTGG |
| | (SEQ ID NO: 14) | (SEQ ID NO: 879) |
| 368 | CDC2 | AGGTATTGTGGTAGTTGG |
| | (SEQ ID NO: 14) | (SEQ ID NO: 880) |
| 369 | CDC2 | GGGTTATTCGATTGGTGA |
| | (SEQ ID NO: 14) | (SEQ ID NO: 881) |
| 370 | CDC2 | GGGTTATTTGATTGGTGA |
| | (SEQ ID NO: 14) | (SEQ ID NO: 882) |
| 371 | CDC2 | AAATTGTTCGTATTTGGT |
| | (SEQ ID NO: 14) | (SEQ ID NO: 883) |
| 372 | CDC2 | AAATTGTTTGTATTTGGT |
| | (SEQ ID NO: 14) | (SEQ ID NO: 884) |
| 373 | ESR1 | AGATATATCGGAGTTTGG |
| | (SEQ ID NO: 28) | (SEQ ID NO: 885) |
| 374 | ESR1 | AGATATATTGGAGTTTGG |
| | (SEQ ID NO: 28) | (SEQ ID NO: 886) |
| 375 | ESR1 | GTTTGGTACGGGGTATAT |
| | (SEQ ID NO: 28) | (SEQ ID NO: 887) |
| 376 | ESR1 | GTTTGGTATGGGGTATAT |
| | (SEQ ID NO: 28) | (SEQ ID NO: 888) |
| 377 | ESR1 | TTTTAAATCGAGTTGTGT |
| | (SEQ ID NO: 28) | (SEQ ID NO: 889) |
| 378 | ESR1 | TTTTAAATTGAGTTGTGT |
| | (SEQ ID NO: 28) | (SEQ ID NO: 890) |
| 379 | ESR1 | TATGAGTTCGGGAGATTA |
| | (SEQ ID NO: 28) | (SEQ ID NO: 891) |
| 380 | ESR1 | TATGAGTTTGGGAGATTA |
| | (SEQ ID NO: 28) | (SEQ ID NO: 892) |
| 381 | ESR1 | TGGAGGTTCGGGAGTTTA |
| | (SEQ ID NO: 28) | (SEQ ID NO: 893) |
| 382 | ESR1 | TGGAGGTTTGGGAGTTTA |
| | (SEQ ID NO: 28) | (SEQ ID NO: 894) |
| 383 | APAF1 | TTTGGTATCGTTTAGAGT |
| | (SEQ ID NO: 3) | (SEQ ID NO: 895) |
| 384 | APAF1 | TTTGGTATTGTTTAGAGT |
| | (SEQ ID NO: 3) | (SEQ ID NO: 896) |
| 385 | APAF1 | GTATGAGTCGTGGTAGGA |
| | (SEQ ID NO: 3) | (SEQ ID NO: 897) |
| 386 | APAF1 | GTATGAGTTGTGGTAGGA |
| | (SEQ ID NO: 3) | (SEQ ID NO: 898) |
| 387 | APAF1 | GTGGATTCGGCGGGATTT |
| | (SEQ ID NO: 3) | (SEQ ID NO: 899) |
| 388 | APAF1 | GTGGATTTGGTGGGATTT |
| | (SEQ ID NO: 3) | (SEQ ID NO: 900) |
| 389 | APAF1 | TTTAGAGGCGGAGAAGAA |
| | (SEQ ID NO: 3) | (SEQ ID NO: 901) |
| 390 | APAF1 | TTTAGAGGTGGAGAAGAA |
| | (SEQ ID NO: 3) | (SEQ ID NO: 902) |
| 391 | APAF1 | GAAGAGGTAGCGAGTGGA |
| | (SEQ ID NO: 3) | (SEQ ID NO: 903) |
| 392 | APAF1 | GAAGAGGTAGTGAGTGGA |
| | (SEQ ID NO: 3) | (SEQ ID NO: 904) |
| 393 | BAK1 | TAGGTTGTCGGTTTGTGC |
| | (SEQ ID NO: 7) | (SEQ ID NO: 905) |
| 394 | BAK1 | TAGGTTGTTGGTTTGTGC |
| | (SEQ ID NO: 7) | (SEQ ID NO: 906) |
| 395 | BAK1 | TTTGTATTCGGTGGTTAT |
| | (SEQ ID NO: 7) | (SEQ ID NO: 907) |
| 396 | BAK1 | TTTGTATTTGGTGGTTAT |
| | (SEQ ID NO: 7) | (SEQ ID NO: 908) |
| 397 | BAK1 | GGAGTTTCGCGGGTTTTT |
| | (SEQ ID NO: 7) | (SEQ ID NO: 909) |
| 398 | BAK1 | GGAGTTTTGTGGGTTTTT |
| | (SEQ ID NO: 7) | (SEQ ID NO: 910) |
| 399 | BAK1 | TAGGATTTCGGTAGGTAA |
| | (SEQ ID NO: 7) | (SEQ ID NO: 911) |
| 400 | BAK1 | TAGGATTTTGGTAGGTAA |
| | (SEQ ID NO: 7) | (SEQ ID NO: 912) |
| 401 | BAX | AGTTTGGGCGTGGGTTAT |
| | (SEQ ID NO: 8) | (SEQ ID NO: 913) |
| 402 | BAX | AGTTTGGGTGTGGGTTAT |
| | (SEQ ID NO: 8) | (SEQ ID NO: 914) |
| 403 | BAX | ATTAGAGTTGCGATTGGA |
| | (SEQ ID NO: 8) | (SEQ ID NO: 915) |
| 404 | BAX | ATTAGAGTTGTGATTGGA |
| | (SEQ ID NO: 8) | (SEQ ID NO: 916) |
| 405 | BAX | GTATTTATCGGGAGATGT |
| | (SEQ ID NO: 8) | (SEQ ID NO: 917) |
| 406 | BAX | GTATTTATTGGGAGATGT |
| | (SEQ ID NO: 8) | (SEQ ID NO: 918) |
| 407 | BAX | TTTAGAGGCGGGGGTGAG |
| | (SEQ ID NO: 8) | (SEQ ID NO: 919) |
| 408 | BAX | TTTAGAGGTGGGGGTGAG |
| | (SEQ ID NO: 8) | (SEQ ID NO: 920) |
| 409 | CASP10 | GAAGTAAACGGTTTTTAG |
| | (SEQ ID NO: 10) | (SEQ ID NO: 921) |
| 410 | CASP10 | GAAGTAAATGGTTTTTAG |
| | (SEQ ID NO: 10) | (SEQ ID NO: 922) |
| 411 | CASP10 | TTATAATACGAAGTTATG |
| | (SEQ ID NO: 10) | (SEQ ID NO: 923) |
| 412 | CASP10 | TTATAATATGAAGTTATG |
| | (SEQ ID NO: 10) | (SEQ ID NO: 924) |
| 413 | CASP10 | GAGGTTTTCGGATTTTTT |
| | (SEQ ID NO: 10) | (SEQ ID NO: 925) |
| 414 | CASP10 | GAGGTTTTTGGATTTTTT |
| | (SEQ ID NO: 10) | (SEQ ID NO: 926) |
| 415 | CASP10 | TTTTGTTTCGGTAAAAGG |
| | (SEQ ID NO: 10) | (SEQ ID NO: 927) |
| 416 | CASP10 | TTTTGTTTTGGTAAAAGG |
| | (SEQ ID NO: 10) | (SEQ ID NO: 928) |
| 417 | CASP8 | GAATGAGTCGAGGAAGGT |
| | (SEQ ID NO: 11) | (SEQ ID NO: 929) |
| 418 | CASP8 | GAATGAGTTGAGGAAGGT |
| | (SEQ ID NO: 11) | (SEQ ID NO: 930) |
| 419 | CASP8 | TATTGAGACGTTAAGTAA |
| | (SEQ ID NO: 11) | (SEQ ID NO: 931) |
| 420 | CASP8 | TATTGAGATGTTAAGTAA |
| | (SEQ ID NO: 11) | (SEQ ID NO: 932) |
| 421 | CASP8 | TAAGGTTACGTAGTTAGT |
| | (SEQ ID NO: 11) | (SEQ ID NO: 933) |
| 422 | CASP8 | TAAGGTTATGTAGTTAGT |
| | (SEQ ID NO: 11) | (SEQ ID NO: 934) |
| 423 | CASP8 | GTTAATAGCGGGGATTTT |
| | (SEQ ID NO: 11) | (SEQ ID NO: 935) |
| 424 | CASP8 | GTTAATAGTGGGGATTTT |
| | (SEQ ID NO: 11) | (SEQ ID NO: 936) |
| 425 | CASP9 | TATATGATCGAGGATATT |
| | (SEQ ID NO: 12) | (SEQ ID NO: 937) |
| 426 | CASP9 | TATATGATTGAGGATATT |
| | (SEQ ID NO: 12) | (SEQ ID NO: 938) |
| 427 | CASP9 | ATTTTTTAGTCGGGTGTT |
| | (SEQ ID NO: 12) | (SEQ ID NO: 939) |
| 428 | CASP9 | ATTTTTTAGTTGGGTGTT |
| | (SEQ ID NO: 12) | (SEQ ID NO: 940) |
| 429 | CASP9 | TTAGTTAGCGTATATTGT |
| | (SEQ ID NO: 12) | (SEQ ID NO: 941) |
| 430 | CASP9 | TTAGTTAGTGTATATTGT |
| | (SEQ ID NO: 12) | (SEQ ID NO: 942) |
| 431 | CASP9 | TGATAAAACGTTAGAGGT |
| | (SEQ ID NO: 12) | (SEQ ID NO: 943) |
| 432 | CASP9 | TGATAAAATGTTAGAGGT |
| | (SEQ ID NO: 12) | (SEQ ID NO: 944) |
| 433 | TCL1A | TTAGAGTATGTTTTTTGG |
| | (SEQ ID NO: 56) | (SEQ ID NO: 945) |
| 434 | TCL1A | TTAGAGTATGTTTTTTGG |
| | (SEQ ID NO: 56) | (SEQ ID NO: 946) |
| 435 | TCL1A | TTTATAGTCGATATTAGG |
| | (SEQ ID NO: 56) | (SEQ ID NO: 947) |
| 436 | TCL1A | TTTATAGTTGATATTAGG |
| | (SEQ ID NO: 56) | (SEQ ID NO: 948) |
| 437 | TCL1A | TAGATTCGGTTTTAGTTG |
| | (SEQ ID NO: 56) | (SEQ ID NO: 949) |
| 438 | TCL1A | TAGATTTGGTTTTAGTTG |
| | (SEQ ID NO: 56) | (SEQ ID NO: 950) |
| 439 | TCL1A | TTTTGGAACGGTGGTAGT |
| | (SEQ ID NO: 56) | (SEQ ID NO: 951) |
| 440 | TCL1A | TTTTGGAATGGTGGTAGT |
| | (SEQ ID NO: 56) | (SEQ ID NO: 952) |
| 441 | PML | TTAAGAGTCGTAGATGTA |
| | (SEQ ID NO: 48) | (SEQ ID NO: 953) |
| 442 | PML | TTAAGAGTTGTAGATGTA |
| | (SEQ ID NO: 48) | (SEQ ID NO: 954) |
| 443 | PML | GAGTTGGGCGTATAAAGA |
| | (SEQ ID NO: 48) | (SEQ ID NO: 955) |
| 444 | PML | GAGTTGGGTGTATAAAGA |
| | (SEQ ID NO: 48) | (SEQ ID NO: 956) |
| 445 | PML | GTAAAGCGGGAGAGGTAG |
| | (SEQ ID NO: 48) | (SEQ ID NO: 957) |
| 446 | PML | GTAAAGTGGGAGAGGTAG |
| | (SEQ ID NO: 48) | (SEQ ID NO: 958) |
| 447 | PML | TTAAAGATACGTTTAGAG |
| | (SEQ ID NO: 48) | (SEQ ID NO: 959) |
| 448 | PML | TTAAAGATATGTTTAGAG |
| | (SEQ ID NO: 48) | (SEQ ID NO: 960) |
| 449 | HOXA5 | AGTTAGTCGGGTTTTAAG |
| | (SEQ ID NO: 35) | (SEQ ID NO: 961) |
| 450 | HOXA5 | AGTTAGTTGGGTTTTAAG |
| | (SEQ ID NO: 35) | (SEQ ID NO: 962) |
| 451 | HOXA5 | TTATAGGGTTCGGTTTTT |
| | (SEQ ID NO: 35) | (SEQ ID NO: 963) |
| 452 | HOXA5 | TTATAGGGTTTGGTTTTT |
| | (SEQ ID NO: 35) | (SEQ ID NO: 964) |
| 453 | HOXA5 | TTTTAAGGCGAGGTTAAA |
| | (SEQ ID NO: 35) | (SEQ ID NO: 965) |
| 454 | HOXA5 | TTTTAAGGTGAGGTTAAA |
| | (SEQ ID NO: 35) | (SEQ ID NO: 966) |
| 455 | HOXA5 | ATGATAGGCGTTTATTAA |
| | (SEQ ID NO: 35) | (SEQ ID NO: 967) |
| 456 | HOXA5 | ATGATAGGTGTTTATTAA |
| | (SEQ ID NO: 35) | (SEQ ID NO: 968) |
| 457 | SDC4 | GGTTATACGATTTTTGTG |
| | (SEQ ID NO: 54) | (SEQ ID NO: 969) |
| 458 | SDC4 | GGTTATATGATTTTTGTG |
| | (SEQ ID NO: 54) | (SEQ ID NO: 970) |
| 459 | SDC4 | TAGGTTTTGCGGGTTATA |
| | (SEQ ID NO: 54) | (SEQ ID NO: 971) |
| 460 | SDC4 | TAGGTTTTGTGGGTTATA |
| | (SEQ ID NO: 54) | (SEQ ID NO: 972) |
| 461 | SDC4 | ATGTGAATCGTGTTAAGA |
| | (SEQ ID NO: 54) | (SEQ ID NO: 973) |
| 462 | SDC4 | ATGTGAATTGTGTTAAGA |
| | (SEQ ID NO: 54) | (SEQ ID NO: 974) |
| 463 | SDC4 | TTTTATATCGGGTGTGTT |
| | (SEQ ID NO: 54) | (SEQ ID NO: 1223) |
| 464 | SDC4 | TTTTATATTGGGTGTGTT |
| | (SEQ ID NO: 54) | (SEQ ID NO: 1224) |
| 465 | PITX2 | TTAAGAGACGGAATAAAG |
| | (SEQ ID NO: 47) | (SEQ ID NO: 975) |
| 466 | PITX2 | TTAAGAGATGGAATAAAG |
| | (SEQ ID NO: 47) | (SEQ ID NO: 976) |
| 467 | PITX2 | AATTAGGACGGGTTGGGA |
| | (SEQ ID NO: 47) | (SEQ ID NO: 977) |
| 468 | PITX2 | AATTAGGATGGGTTGGGA |
| | (SEQ ID NO: 47) | (SEQ ID NO: 978) |
| 469 | PITX2 | TAGGAGTACGGATTTTTA |
| | (SEQ ID NO: 47) | (SEQ ID NO: 979) |
| 470 | PITX2 | TAGGAGTATGGATTTTTA |
| | (SEQ ID NO: 47) | (SEQ ID NO: 980) |
| 471 | PITX2 | GAGTTAATCGAGGAGTAG |
| | (SEQ ID NO: 47) | (SEQ ID NO: 981) |
| 472 | PITX2 | GAGTTAATTGAGGAGTAG |
| | (SEQ ID NO: 47) | (SEQ ID NO: 982) |
| 473 | PITX2 | GGATTTGGCGGGGTATTT |
| | (SEQ ID NO: 47) | (SEQ ID NO: 983) |
| 474 | PITX2 | GGATTTGGTGGGGTATTT |
| | (SEQ ID NO: 47) | (SEQ ID NO: 984) |
| 475 | GPR37 | AGTTTAGACGAAGTTTTA |
| | (SEQ ID NO: 31) | (SEQ ID NO: 985) |
| 476 | GPR37 | AGTTTAGATGAAGTTTTA |
| | (SEQ ID NO: 31) | (SEQ ID NO: 986) |
| 477 | GPR37 | GTTATTTTCGGTGGTTGA |
| | (SEQ ID NO: 31) | (SEQ ID NO: 987) |
| 478 | GPR37 | GTTATTTTTGGTGGTTGA |
| | (SEQ ID NO: 31) | (SEQ ID NO: 988) |
| 479 | GPR37 | AGGGTGATAGCGGAAGAT |
| | (SEQ ID NO: 31) | (SEQ ID NO: 989) |
| 480 | GPR37 | AGGGTGATAGTGGAAGAT |
| | (SEQ ID NO: 31) | (SEQ ID NO: 990) |
| 481 | GPR37 | GTTAGGATCGATATTTGT |
| | (SEQ ID NO: 31) | (SEQ ID NO: 991) |
| 482 | GPR37 | GTTAGGATTGATATTTGT |
| | (SEQ ID NO: 31) | (SEQ ID NO: 992) |
| 483 | PRAME | GGGATTGTCGTATAAAAT |
| | (SEQ ID NO: 50) | (SEQ ID NO: 993) |
| 484 | PRAME | GGGATTGTTGTATAAAAT |
| | (SEQ ID NO: 50) | (SEQ ID NO: 994) |
| 485 | PRAME | TTTAGATTCGATTTGGGA |
| | (SEQ ID NO: 50) | (SEQ ID NO: 995) |
| 486 | PRAME | TTTAGATTTGATTTGGGA |
| | (SEQ ID NO: 50) | (SEQ ID NO: 996) |
| 487 | PRAME | TAGAGTTGCGGGTTTTTA |
| | (SEQ ID NO: 50) | (SEQ ID NO: 997) |
| 488 | PRAME | TAGAGTTGTGGGTTTTTA |
| | (SEQ ID NO: 50) | (SEQ ID NO: 998) |
| 489 | PRAME | AATAGGTCGCGTGGTGAG |
| | (SEQ ID NO: 50) | (SEQ ID NO: 999) |
| 490 | PRAME | AATAGGTTGTGTGGTGAG |
| | (SEQ ID NO: 50) | (SEQ ID NO: 1000) |
| 491 | N-MYC | AGTTTTGGCGTTTTTTTT |
| | (SEQ ID NO: 60) | (SEQ ID NO: 1001) |
| 492 | N-MYC | AGTTTTGGTGTTTTTTTT |
| | (SEQ ID NO: 60) | (SEQ ID NO: 1002) |
| 493 | N-MYC | GGTAAAGTCGTTTTTTTT |
| | (SEQ ID NO: 60) | (SEQ ID NO: 1003) |
| 494 | N-MYC | GGTAAAGTTGTTTTTTTT |
| | (SEQ ID NO: 60) | (SEQ ID NO: 1004) |
| 495 | N-MYC | TTTGTTTGCGTTATAGTT |
| | (SEQ ID NO: 60) | (SEQ ID NO: 1005) |
| 496 | N-MYC | TTTGTTTGTGTTTATAGTT |
| | (SEQ ID NO: 60) | (SEQ ID NO: 1006) |
| 497 | N-MYC | ATATTTTTCGAGTTTTAA |
| | (SEQ ID NO: 60) | (SEQ ID NO: 1007) |
| 498 | N-MYC | ATATTTTTTGAGTTTTAA |
| | (SEQ ID NO: 60) | (SEQ ID NO: 1008) |
| 499 | N-MYC | TTTTAAAGCGTAGGTTGT |
| | (SEQ ID NO: 60) | (SEQ ID NO: 1009) |
| 500 | N-MYC | TTTTAAAGTGTAGGTTGT |
| | (SEQ ID NO: 60) | (SEQ ID NO: 1010) |
| 501 | N-MYC | GGTGGATGCGGGGGGTTT |
| | (SEQ ID NO: 60) | (SEQ ID NO: 1011) |
| 502 | N-MYC | GGTGGATGTGGGGGGTTT |
| | (SEQ ID NO: 60) | (SEQ ID NO: 1012) |
| 503 | L-MYC | GGGTGGGGCGGGGAGTAG |
| | (SEQ ID NO: 61) | (SEQ ID NO: 1013) |
| 504 | L-MYC | GGGTGGGGTGGGGAGTAG |
| | (SEQ ID NO: 61) | (SEQ ID NO: 1014) |
| 505 | L-MYC | TTTTAGTTCGGAGTGGGT |
| | (SEQ ID NO: 61) | (SEQ ID NO: 1015) |
| 506 | L-MYC | TTTTAGTTTGGAGTGGGT |
| | (SEQ ID NO: 61) | (SEQ ID NO: 1016) |
| 507 | L-MYC | AAGGAGTACGGTTTAGTT |
| | (SEQ ID NO: 61) | (SEQ ID NO: 1017) |
| 508 | L-MYC | AAGGAGTATGGTTTAGTT |
| | (SEQ ID NO: 61) | (SEQ ID NO: 1018) |
| 509 | L-MYC | AGTTTAGTCGGTTGGTAT |
| | (SEQ ID NO: 61) | (SEQ ID NO: 1019) |
| 510 | L-MYC | AGTTTAGTTGGTTGGTAT |
| | (SEQ ID NO: 61) | (SEQ ID NO: 1020) |
| 511 | L-MYC | TTTGTTTGCGTTTTTTAG |
| | (SEQ ID NO: 61) | (SEQ ID NO: 1021) |
| 512 | L-MYC | TTTGTTTGTGTTTTTTAG |
| | (SEQ ID NO: 61) | (SEQ ID NO: 1022) |
| 513 | L-MYC | TTTTTTAGCGTGTAATTT |
| | (SEQ ID NO: 61) | (SEQ ID NO: 1023) |
| 514 | L-MYC | TTTTTTAGTGTGTAATTT |
| | (SEQ ID NO: 61) | (SEQ ID NO: 1024) |
| 515 | L-MYC | GGGGTTATCGGGGATTGA |
| | (SEQ ID NO: 61) | (SEQ ID NO: 1025) |
| 516 | L-MYC | GGGGTTATTGGGGATTGA |
| | (SEQ ID NO: 61) | (SEQ ID NO: 1026) |
| 517 | L-MYC | GGTGGGGTCGGTTTTATT |
| | (SEQ ID NO: 61) | (SEQ ID NO: 1027) |
| 518 | L-MYC | GGTGGGGTTGGTTTTATT |
| | (SEQ ID NO: 61) | (SEQ ID NO: 1028) |
| 519 | L-MYC | GGAGAGGGCGGGGTTTGT |
| | (SEQ ID NO: 61) | (SEQ ID NO: 1029) |
| 520 | L-MYC | GGAGAGGGTGGGGTTTGT |
| | (SEQ ID NO: 61) | (SEQ ID NO: 1030) |
| 521 | L-MYC | TTGAGGGTCGTTAGGTGG |
| | (SEQ ID NO: 61) | (SEQ ID NO: 1031) |
| 522 | L-MYC | TTGAGGGTTGTTAGGTGG |
| | (SEQ ID NO: 61) | (SEQ ID NO: 1032) |
| 523 | C-ABL | ATTAGAGTCGGGAGGGGC |
| | (SEQ ID NO: 62) | (SEQ ID NO: 1033) |
| 524 | C-ABL | ATTAGAGTTGGGAGGGGC |
| | (SEQ ID NO: 62) | (SEQ ID NO: 1034) |
| 525 | C-ABL | GGGGTATTCGGGTTTTTT |
| | (SEQ ID NO: 62) | (SEQ ID NO: 1035) |
| 526 | C-ABL | GGGGTATTTGGGTTTTTT |
| | (SEQ ID NO: 62) | (SEQ ID NO: 1036) |
| 527 | C-ABL | TTTGGTTTCGTTGTGGAG |
| | (SEQ ID NO: 62) | (SEQ ID NO: 1037) |
| 528 | C-ABL | TTTGGTTTTGTTGTGGAG |
| | (SEQ ID NO: 62) | (SEQ ID NO: 1038) |
| 529 | C-ABL | TTGAGTAGCGTTGGAGTC |
| | (SEQ ID NO: 62) | (SEQ ID NO: 1039) |
| 530 | C-ABL | TTGAGTAGTGTTGGAGTC |
| | (SEQ ID NO: 62) | (SEQ ID NO: 1040) |
| 531 | C-ABL | GGGTTTTGCGGTTGAGGA |
| | (SEQ ID NO: 62) | (SEQ ID NO: 1041) |
| 532 | C-ABL | GGGTTTTGTGGTTGAGGA |
| | (SEQ ID NO: 62) | (SEQ ID NO: 1042) |
| 533 | C-ABL | AGTTTTTTCGTTGTTTAC |
| | (SEQ ID NO: 62) | (SEQ ID NO: 1043) |
| 534 | C-ABL | AGTTTTTTTGTTGTTTAC |
| | (SEQ ID NO: 62) | (SEQ ID NO: 1044) |
| 535 | C-ABL | GTTGTTTACGGTTTTTAT |
| | (SEQ ID NO: 62) | (SEQ ID NO: 1045) |
| 536 | C-ABL | GTTGTTTATGGTTTTTAT |
| | (SEQ ID NO: 62) | (SEQ ID NO: 1046) |
| 537 | C-ABL | TTTGAGGGCGGTGGTGGT |
| | (SEQ ID NO: 62) | (SEQ ID NO: 1047) |
| 538 | C-ABL | TTTGAGGGTGGTGGTGGT |
| | (SEQ ID NO: 62) | (SEQ ID NO: 1048) |
| 539 | C-ABL | GGGTGTTTCGGGTAGAGA |
| | (SEQ ID NO: 62) | (SEQ ID NO: 1049) |
| 540 | C-ABL | GGGTGTTTTGGGTAGAGA |
| | (SEQ ID NO: 62) | (SEQ ID NO: 1050) |
| 541 | C-ABL | GAGATTTTCGGGTTTGGG |
| | (SEQ ID NO: 62) | (SEQ ID NO: 1051) |
| 542 | C-ABL | GAGATTTTTGGGTTTGGG |
| | (SEQ ID NO: 62) | (SEQ ID NO: 1052) |
| 543 | ELK1 | TTTGTTTTCGTTGAGTAG |
| | (SEQ ID NO: 63) | (SEQ ID NO: 1053) |
| 544 | ELK1 | TTTGTTTTTGTTGAGTAG |
| | (SEQ ID NO: 63) | (SEQ ID NO: 1054) |
| 545 | ELK1 | TTTATTTTCGTTTTTGGG |
| | (SEQ ID NO: 63) | (SEQ ID NO: 1241) |
| 546 | ELK1 | TTTATTTTTGTTTTTGGG |
| | (SEQ ID NO: 63) | (SEQ ID NO: 1242) |
| 547 | ELK1 | ATAGGTTTCGAGTTTTTT |
| | (SEQ ID NO: 63) | (SEQ ID NO: 1055) |
| 548 | ELK1 | ATAGGTTTTGAGTTTTTT |
| | (SEQ ID NO: 63) | (SEQ ID NO: 1056) |
| 549 | ELK1 | TTATAGTTCGTTAGTTTT |
| | (SEQ ID NO: 63) | (SEQ ID NO: 1057) |
| 550 | ELK1 | TTATAGTTTGTTAGTTTT |
| | (SEQ ID NO: 63) | (SEQ ID NO: 1058) |
| 551 | ELK1 | TGGAGTATCGTGAAGGTC |
| | (SEQ ID NO: 63) | (SEQ ID NO: 1059) |
| 552 | ELK1 | TGGAGTATTGTGAAGGTC |
| | (SEQ ID NO: 63) | (SEQ ID NO: 1060) |
| 553 | ELK1 | TTTTTTTACGTTAATTAC |
| | (SEQ ID NO: 63) | (SEQ ID NO: 1061) |
| 554 | ELK1 | TTTTTTTATGTTAATTAC |
| | (SEQ ID NO: 63) | (SEQ ID NO: 1062) |
| 555 | ELK1 | GTTAATTACGGTATAGTT |
| | (SEQ ID NO: 63) | (SEQ ID NO: 1063) |
| 556 | ELK1 | GTTAATTATGGTATAGTT |
| | (SEQ ID NO: 63) | (SEQ ID NO: 1064) |
| 557 | ELK1 | GAAGGGTTCGTTTTTTAA |
| | (SEQ ID NO: 63) | (SEQ ID NO: 1065) |
| 558 | ELK1 | GAAGGGTTTGTTTTTTAA |
| | (SEQ ID NO: 63) | (SEQ ID NO: 1066) |
| 559 | ELK1 | TAATTTTTCGAGGTTTTG |
| | (SEQ ID NO: 63) | (SEQ ID NO: 1067) |
| 560 | ELK1 | TAATTTTTTGAGGTTTTG |
| | (SEQ ID NO: 63) | (SEQ ID NO: 1068) |
| 561 | ELK1 | ATTAATAGCGTTTTGGTT |
| | (SEQ ID NO: 63) | (SEQ ID NO: 1069) |
| 562 | ELK1 | ATTAATAGTGTTTTGGTT |
| | (SEQ ID NO: 63) | (SEQ ID NO: 1070) |
| 563 | ELK1 | GTTTAGTTCGATTTATTT |
| | (SEQ ID NO: 63) | (SEQ ID NO: 1071) |
| 564 | ELK1 | GTTTAGTTTGATTTATTT |
| | (SEQ ID NO: 63) | (SEQ ID NO: 1072) |
| 565 | ELK1 | GGTTGTTACGTTTATTTT |
| | (SEQ ID NO: 63) | (SEQ ID NO: 1073) |
| 566 | ELK1 | GGTTGTTATGTTTATTTT |
| | (SEQ ID NO: 63) | (SEQ ID NO: 1074) |
| 567 | Tubulin | TACTTCAACGTCCTAATA |
| | (SEQ ID NO: 64) | (SEQ ID NO: 1239) |
| 568 | Tubulin | TACTTCAACATCCTAATA |
| | (SEQ ID NO: 64) | (SEQ ID NO: 1240) |
| 569 | Tubulin | CAATACTCCGTCCATTAA |
| | (SEQ ID NO: 64) | (SEQ ID NO: 1243) |
| 570 | Tubulin | CAATACTCCATCCATTAA |
| | (SEQ ID NO: 64) | (SEQ ID NO: 1244) |
| 571 | Tubulin | TAATCAACCGCAACCTCC |
| | (SEQ ID NO: 64) | (SEQ ID NO: 1075) |
| 572 | Tubulin | TAATCAACCACAACCTCC |
| | (SEQ ID NO: 64) | (SEQ ID NO: 1076) |
| 573 | Tubulin | CTTACCCCCGTCAATCAA |
| | (SEQ ID NO: 64) | (SEQ ID NO: 1077) |
| 574 | Tubulin | CTTACCCCCATCAATCAA |
| | (SEQ ID NO: 64) | (SEQ ID NO: 1078) |
| 575 | Tubulin | GCTAATCTCGAAATAAAC |
| | (SEQ ID NO: 64) | (SEQ ID NO: 1079) |
| 576 | Tubulin | GCTAATCTCAAAATAAAC |
| | (SEQ ID NO: 64) | (SEQ ID NO: 1080) |
| 577 | CSF1 | TTATAGAGCGTTAGTATT |
| | (SEQ ID NO: 65) | (SEQ ID NO: 1081) |
| 578 | CSF1 | TTATAGAGTGTTAGTATT |
| | (SEQ ID NO: 65) | (SEQ ID NO: 1082) |
| 579 | CSF1 | AAGTGTAGCGTAGAAGAT |
| | (SEQ ID NO: 65) | (SEQ ID NO: 1083) |
| 580 | CSF1 | AAGTGTAGTGTAGAAGAT |
| | (SEQ ID NO: 65) | (SEQ ID NO: 1084) |
| 581 | CSF1 | AGGGGAGGCGGGGGAAGG |
| | (SEQ ID NO: 65) | (SEQ ID NO: 1085) |
| 582 | CSF1 | AGGGGAGGTGGGGGAAGG |
| | (SEQ ID NO: 65) | (SEQ ID NO: 1086) |
| 583 | CSF1 | GGGGAAGGCGGTTGAGTG |
| | (SEQ ID NO: 65) | (SEQ ID NO: 1087) |
| 584 | CSF1 | GGGGAAGGTGGTTGAGTG |
| | (SEQ ID NO: 65) | (SEQ ID NO: 1088) |
| 585 | CSF1 | GTGTTTGGCGTTTGGTTA |
| | (SEQ ID NO: 65) | (SEQ ID NO: 1089) |
| 586 | CSF1 | GTGTTTGGTGTTTGGTTA |
| | (SEQ ID NO: 65) | (SEQ ID NO: 1090) |
| 587 | CD1R3 | TAACTATACGAACTACAA |
| | (SEQ ID NO: 66) | (SEQ ID NO: 1091) |
| 588 | CD1R3 | TAACTATACAAACTACAA |
| | (SEQ ID NO: 66) | (SEQ ID NO: 1092) |
| 589 | CD1R3 | ACCCTCCTCGCTAAACTT |
| | (SEQ ID NO: 66) | (SEQ ID NO: 1093) |
| 590 | CD1R3 | ACCCTCCTCACTAAACTT |
| | (SEQ ID NO: 66) | (SEQ ID NO: 1094) |
| 591 | CD1R3 | CCAAACCCCGACAACCCT |
| | (SEQ ID NO: 66) | (SEQ ID NO: 1095) |
| 592 | CD1R3 | CCAAACGCCAACAACCCT |
| | (SEQ ID NO: 66) | (SEQ ID NO: 1096) |
| 593 | CD1R3 | TCCCTTTCCGCTAAATAC |
| | (SEQ ID NO: 66) | (SEQ ID NO: 1097) |
| 594 | CD1R3 | TCCCTTTCCACTAAATAC |
| | (SEQ ID NO: 66) | (SEQ ID NO: 1098) |
| 595 | CD1R3 | TCAACTCACGTCCCTTTC |
| | (SEQ ID NO: 66) | (SEQ ID NO: 1099) |
| 596 | CD1R3 | TCAACTCACATCCCTTTC |
| | (SEQ ID NO: 66) | (SEQ ID NO: 1100) |
| 597 | CSNK2B | AGGAGTTTCGGAGGAAAT |
| | (SEQ ID NO: 67) | (SEQ ID NO: 1235) |
| 598 | CSNK2B | AGGAGTTTTGGAGGAAAT |
| | (SEQ ID NO: 67) | (SEQ ID NO: 1236) |
| 599 | CSNK2B | ATTTCCTCCGAAACTCCT |
| | (SEQ ID NO: 67) | (SEQ ID NO: 1237) |
| 600 | CSNK2B | ATTTCCTCCAAAACTCCT |
| | (SEQ ID NO: 67) | (SEQ ID NO: 1238) |
| 601 | CSNK2B | GATTGATTCGTATTAGGG |
| | (SEQ ID NO: 67) | (SEQ ID NO: 1101) |
| 602 | CSNK2B | GATTGATTTGTATTAGGG |
| | (SEQ ID NO: 67) | (SEQ ID NO: 1102) |
| 603 | CSNK2B | CCCTAATACGAATCAATC |
| | (SEQ ID NO: 67) | (SEQ ID NO: 1103) |
| 604 | CSNK2B | CCCTAATACAAATCAATC |
| | (SEQ ID NO: 67) | (SEQ ID NO: 1104) |
| 605 | CSNK2B | GGATTTAGCGGATTGGTC |
| | (SEQ ID NO: 67) | (SEQ ID NO: 1105) |
| 606 | CSNK2B | GGATTTAGTGGATTGGTC |
| | (SEQ ID NO: 67) | (SEQ ID NO: 1106) |
| 607 | CSNK2B | GACCAATCCGCTAAATCC |
| | (SEQ ID NO: 67) | (SEQ ID NO: 1107) |
| 608 | CSNK2B | GACCAATCCACTAAATCC |
| | (SEQ ID NO: 67) | (SEQ ID NO: 1108) |
| 609 | CSNK2B | GGATTGGTCGAGGATAGG |
| | (SEQ ID NO: 67) | (SEQ ID NO: 1109) |
| 610 | CSNK2B | GGATTGGTTGAGGATAGG |
| | (SEQ ID NO: 67) | (SEQ ID NO: 1110) |
| 611 | CSNK2B | CCTATCCTCGACCAATCC |
| | (SEQ ID NO: 67) | (SEQ ID NO: 1111) |
| 612 | CSNK2B | CCTATCCTCAACCAATCC |
| | (SEQ ID NO: 67) | (SEQ ID NO: 1112) |
| 613 | CSNK2B | GAGGGGAACGTGAGGAGA |
| | (SEQ ID NO: 67) | (SEQ ID NO: 1113) |
| 614 | CSNK2B | GAGGGGAATGTGAGGAGA |
| | (SEQ ID NO: 67) | (SEQ ID NO: 1114) |
| 615 | CSNK2B | TCTCCTCACGTTCCCCTC |
| | (SEQ ID NO: 67) | (SEQ ID NO: 1115) |
| 616 | CSNK2B | TCTCCTCACATTCCCCTC |
| | (SEQ ID NO: 67) | (SEQ ID NO: 1116) |
| 617 | CSNK2B | TAGGTTAGCGTATTGGGA |
| | (SEQ ID NO: 67) | (SEQ ID NO: 1117) |
| 618 | CSNK2B | TAGGTTAGTGTATTGGGA |
| | (SEQ ID NO: 67) | (SEQ ID NO: 1118) |
| 619 | CSNK2B | TCCCAATACGCTAACCTA |
| | (SEQ ID NO: 67) | (SEQ ID NO: 1119) |
| 620 | CSNK2B | TCCCAATACACTAACCTA |
| | (SEQ ID NO: 67) | (SEQ ID NO: 1120) |
| 621 | CSNK2B | TATTGGGACGTTTTAGTT |
| | (SEQ ID NO: 67) | (SEQ ID NO: 1121) |
| 622 | CSNK2B | TATTGGGATGTTTTAGTT |
| | (SEQ ID NO: 67) | (SEQ ID NO: 1122) |
| 623 | CSNK2B | AACTAAAACGTCCCAATA |
| | (SEQ ID NO: 67) | (SEQ ID NO: 1123) |
| 624 | CSNK2B | AACTAAAACATCCCAATA |
| | (SEQ ID NO: 67) | (SEQ ID NO: 1124) |
| 625 | Me491/TD63 | TTGTTATTCGGTTTTAGT |
| | (SEQ ID NO: 68) | (SEQ ID NO: 1125) |
| 626 | Me491/TD63 | TTGTTATTTGGTTTTAGT |
| | (SEQ ID NO: 68) | (SEQ ID NO: 1126) |
| 627 | Me491/TD63 | AGTTGGTACGGAGGTATA |
| | (SEQ ID NO: 68) | (SEQ ID NO: 1127) |
| 628 | Me491/TD63 | AGTTGGTATGGAGGTATA |
| | (SEQ ID NO: 68) | (SEQ ID NO: 1128) |
| 629 | Me491/TD63 | AGGTATAGCGTTAGAGGG |
| | (SEQ ID NO: 68) | (SEQ ID NO: 1129) |
| 630 | Me491/TD63 | AGGTATAGTGTTAGAGGG |
| | (SEQ ID NO: 68) | (SEQ ID NO: 1130) |
| 631 | Me491/TD63 | AGGGGTTACGTGGTTTTG |
| | (SEQ ID NO: 68) | (SEQ ID NO: 1131) |
| 632 | Me491/TD63 | AGGGGTTATGTGGTTTTG |
| | (SEQ ID NO: 68) | (SEQ ID NO: 1132) |
| 633 | Me491/TD63 | TGTATAGTCGGATGGGGA |
| | (SEQ ID NO: 68) | (SEQ ID NO: 1133) |
| 634 | Me491/TD63 | TGTATAGTTGGATGGGGA |
| | (SEQ ID NO: 68) | (SEQ ID NO: 1134) |
| 635 | AR | ACCTCTCTCGAAATAACA |
| | (SEQ ID NO: 69) | (SEQ ID NO: 1135) |
| 636 | AR | ACCTCTCTCAAAATAACA |
| | (SEQ ID NO: 69) | (SEQ ID NO: 1136) |
| 637 | AR | CTCTAAAACGCAACCTCT |
| | (SEQ ID NO: 69) | (SEQ ID NO: 1137) |
| 638 | AR | CTCTAAAACACAACCTCT |
| | (SEQ ID NO: 69) | (SEQ ID NO: 1138) |
| 639 | AR | AACTACTACGACAACCCC |
| | (SEQ ID NO: 69) | (SEQ ID NO: 1139) |
| 640 | AR | AACTACTACAACAACCCC |
| | (SEQ ID NO: 69) | (SEQ ID NO: 1140) |
| 641 | AR | ACTAACCTCGCTCAAAAT |
| | (SEQ ID NO: 69) | (SEQ ID NO: 1141) |
| 642 | AR | ACTAACGTCACTCAAAAT |
| | (SEQ ID NO: 69) | (SEQ ID NO: 1142) |
| 643 | AR | ACTACCTTCGAATACTAC |
| | (SEQ ID NO: 69) | (SEQ ID NO: 1257) |
| 644 | AR | ACTACCTTCAAATACTAC |
| | (SEQ ID NO: 69) | (SEQ ID NO: 1258) |
| 645 | CDK4 | TATGGGGTCGTAGGAATC |
| | (SEQ ID NO: 70) | (SEQ ID NO: 1143) |
| 646 | CDK4 | TATGGGGTTGTAGGAATC |
| | (SEQ ID NO: 70) | (SEQ ID NO: 1144) |
| 647 | CDK4 | GATTCCTACGACCCCATA |
| | (SEQ ID NO: 70) | (SEQ ID NO: 1145) |
| 648 | CDK4 | GATTCCTACAACCCCATA |
| | (SEQ ID NO: 70) | (SEQ ID NO: 1146) |
| 649 | CDK4 | GGAAGGGTCGTTTAAGGG |
| | (SEQ ID NO: 70) | (SEQ ID NO: 1147) |
| 650 | CDK4 | GGAAGGGTTGTTTAAGGG |
| | (SEQ ID NO: 70) | (SEQ ID NO: 1148) |
| 651 | CDK4 | CCCTTAAACGACCCTTCC |
| | (SEQ ID NO: 70) | (SEQ ID NO: 1149) |
| 652 | CDK4 | CCCTTAAACAACCCTTCC |
| | (SEQ ID NO: 70) | (SEQ ID NO: 1150) |
| 653 | CDK4 | TTTAAGGGCGGGAAGTGG |
| | (SEQ ID NO: 70) | (SEQ ID NO: 1247) |
| 654 | CDK4 | TTTAAGGGTGGGAAGTGG |
| | (SEQ ID NO: 70) | (SEQ ID NO: 1248) |
| 655 | CDK4 | CCACTTCCCGCCGTTAAA |
| | (SEQ ID NO: 70) | (SEQ ID NO: 1249) |
| 656 | CDK4 | CCACTTCCCACCCTTAAA |
| | (SEQ ID NO: 70) | (SEQ ID NO: 1250) |
| 657 | CDK4 | AGGATTTTCGATGTAAGG |
| | (SEQ ID NO: 70) | (SEQ ID NO: 1151) |
| 658 | CDK4 | AGGATTTTTGATGTAAGG |
| | (SEQ ID NO: 70) | (SEQ ID NO: 1152) |
| 659 | CDK4 | CCTTACATCGAAAATCCT |
| | (SEQ ID NO: 70) | (SEQ ID NO: 1153) |
| 660 | CDK4 | CCTTACATCAAAAATCCT |
| | (SEQ ID NO: 70) | (SEQ ID NO: 1154) |
| 661 | CDK4 | GGGTTTTACGTGGTTGGA |
| | (SEQ ID NO: 70) | (SEQ ID NO: 1231) |
| 662 | CDK4 | GGGTTTTATGTGGTTGGA |
| | (SEQ ID NO: 70) | (SEQ ID NO: 1232) |
| 663 | CDK4 | TCCAACCACGTAAAACCC |
| | (SEQ ID NO: 70) | (SEQ ID NO: 1233) |
| 664 | CDK4 | TCCAACCACATAAAACCC |
| | (SEQ ID NO: 70) | (SEQ ID NO: 1234) |
| 665 | Humos | CCTTACTACGTTAAACTC |
| | (SEQ ID NO: 71) | (SEQ ID NO: 1155) |
| 666 | Humos | CCTTACTACATTAAACTC |
| | (SEQ ID NO: 71) | (SEQ ID NO: 1156) |
| 667 | Humos | GTTACCACCGAACTCCAT |
| | (SEQ ID NO: 71) | (SEQ ID NO: 1245) |
| 668 | Humos | GTTACCACCAAACTCCAT |
| | (SEQ ID NO: 71) | (SEQ ID NO: 1246) |
| 669 | Humos | CTCCCCTACGTCCCCCTC |
| | (SEQ ID NO: 71) | (SEQ ID NO: 1253) |
| 670 | Humos | CTCCCCTACATCCCCCTC |
| | (SEQ ID NO: 71) | (SEQ ID NO: 1254) |
| 671 | Humos | CTCCAATACGACAATAAA |
| | (SEQ ID NO: 71) | (SEQ ID NO: 1157) |
| 672 | Humos | CTCCAATACAACAATAAA |
| | (SEQ ID NO: 71) | (SEQ ID NO: 1158) |
| 673 | Humos | AAACAAACCGTTCACAAC |
| | (SEQ ID NO: 71) | (SEQ ID NO: 1251) |
| 674 | Humos | AAACAAACCATTCACAAC |
| | (SEQ ID NO: 71) | (SEQ ID NO: 1252) |
| 675 | CDC25A | GTGTAGGTCGGTTTGGTT |
| | (SEQ ID NO: 72) | (SEQ ID NO: 1159) |
| 676 | CDC25A | GTGTAGGTTGGTTTGGTT |
| | (SEQ ID NO: 72) | (SEQ ID NO: 1160) |
| 677 | CDC25A | AACCAAACCGACCTACAC |
| | (SEQ ID NO: 72) | (SEQ ID NO: 1161) |
| 678 | CDC25A | AACCAAACCAACCTACAC |
| | (SEQ ID NO: 72) | (SEQ ID NO: 1162) |
| 679 | CDC25A | TTGTTATTCGGAGTTGGG |
| | (SEQ ID NO: 72) | (SEQ ID NO: 1163) |
| 680 | CDC25A | TTGTTATTTGGAGTTGGG |
| | (SEQ ID NO: 72) | (SEQ ID NO: 1164) |
| 681 | CDC25A | CCCAACTCCGAATAACAA |
| | (SEQ ID NO: 72) | (SEQ ID NO: 1165) |
| 682 | CDC25A | CCCAACTCCAAATAACAA |
| | (SEQ ID NO: 72) | (SEQ ID NO: 1166) |
| 683 | CDC25A | TGGGTAAGCGGGTGGGAG |
| | (SEQ ID NO: 72) | (SEQ ID NO: 1167) |
| 684 | CDC25A | TGGGTAAGTGGGTGGGAG |
| | (SEQ ID NO: 72) | (SEQ ID NO: 1168) |
| 685 | CDC25A | CTCCCACCCGCTTACCCA |
| | (SEQ ID NO: 72) | (SEQ ID NO: 1169) |
| 686 | CDC25A | CTCCCACCCACTTACCCA |
| | (SEQ ID NO: 72) | (SEQ ID NO: 1170) |
| 687 | CDC25A | GAGAATAGCGAAGATAGC |
| | (SEQ ID NO: 72) | (SEQ ID NO: 1171) |
| 688 | CDC25A | GAGAATAGTGAAGATAGC |
| | (SEQ ID NO: 72) | (SEQ ID NO: 1172) |
| 689 | CDC25A | GCTATCTTCGCTATTCTC |
| | (SEQ ID NO: 72) | (SEQ ID NO: 1173) |
| 690 | CDC25A | GCTATCTTCACTATTCTC |
| | (SEQ ID NO: 72) | (SEQ ID NO: 1174) |
| 691 | CDC25A | TATTGAGTCGTTATTATC |
| | (SEQ ID NO: 72) | (SEQ ID NO: 1175) |
| 692 | CDC25A | TATTGAGTTGTTATTATC |
| | (SEQ ID NO: 72) | (SEQ ID NO: 1176) |
| 693 | CDC25A | GATAATAACGACTCAATA |
| | (SEQ ID NO: 72) | (SEQ ID NO: 1177) |
| 694 | CDC25A | GATAATAACAACTCAATA |
| | (SEQ ID NO: 72) | (SEQ ID NO: 1178) |
| 695 | CMYCex3 | GAAGAAATCGATGTTGTT |
| | (SEQ ID NO: 73) | (SEQ ID NO: 1179) |
| 696 | CMYCex3 | GAAGAAATTGATGTTGTT |
| | (SEQ ID NO: 73) | (SEQ ID NO: 1180) |
| 697 | CMYCex3 | AGGTGTTACGTTTTTATA |
| | (SEQ ID NO: 73) | (SEQ ID NO: 1181) |
| 698 | CMYCex3 | AGGTGTTATGTTTTTATA |
| | (SEQ ID NO: 73) | (SEQ ID NO: 1182) |
| 699 | CMYCex3 | TTTTTATTCGGAAGGATT |
| | (SEQ ID NO: 73) | (SEQ ID NO: 1183) |
| 700 | CMYCex3 | TTTTTATTTGGAAGGATT |
| | (SEQ ID NO: 73) | (SEQ ID NO: 1184) |
| 701 | CMYCex3 | GTAATAATCGAAAATGTA |
| | (SEQ ID NO: 73) | (SEQ ID NO: 1185) |
| 702 | CMYCex3 | GTAATAATTGAAAATGTA |
| | (SEQ ID NO: 73) | (SEQ ID NO: 1186) |
| 703 | CMYCex3 | TTAAGAGGCGAATATATA |
| | (SEQ ID NO: 73) | (SEQ ID NO: 1187) |
| 704 | CMYCex3 | TTAAGAGGTGAATATATA |
| | (SEQ ID NO: 73) | (SEQ ID NO: 1188) |
| 705 | CMYCex3 | ATATATAACGTTTTGGAG |
| | (SEQ ID NO: 73) | (SEQ ID NO: 1189) |
| 706 | CMYCex3 | ATATATAATGTTTTGGAG |
| | (SEQ ID NO: 73) | (SEQ ID NO: 1190) |
| 707 | CMYCex3 | TTTTGGAGCGTTAGAGGA |
| | (SEQ ID NO: 73) | (SEQ ID NO: 1191) |
| 708 | CMYCex3 | TTTTGGAGTGTTAGAGGA |
| | (SEQ ID NO: 73) | (SEQ ID NO: 1192) |
| 709 | CMYCex3 | AGGAGGAACGAGTTAAAA |
| | (SEQ ID NO: 73) | (SEQ ID NO: 1193) |
| 710 | CMYCex3 | AGGAGGAATGAGTTAAAA |
| | (SEQ ID NO: 73) | (SEQ ID NO: 1194) |
| 711 | CMYCex3 | AGTTAAAACGGAGTTTTT |
| | (SEQ ID NO: 73) | (SEQ ID NO: 1195) |
| 712 | CMYCex3 | AGTTAAAATGGAGTTTTT |
| | (SEQ ID NO: 73) | (SEQ ID NO: 1196) |
| 713 | CMYCex3 | TTGTTTTGCGTGATTAGA |
| | (SEQ ID NO: 73) | (SEQ ID NO: 1197) |
| 714 | CMYCex3 | TTGTTTTGTGTGATTAGA |
| | (SEQ ID NO: 73) | (SEQ ID NO: 1198) |
| 715 | CMYCex3 | TTAGATTTCGGAGTTGGA |
| | (SEQ ID NO: 73) | (SEQ ID NO: 1199) |
| 716 | CMYCex3 | TTAGATTTTGGAGTTGGA |
| | (SEQ ID NO: 73) | (SEQ ID NO: 1200) |
| 717 | CMYCex3 | ATTTTGTTCGTTTAAGTA |
| | (SEQ ID NO: 73) | (SEQ ID NO: 1201) |
| 718 | CMYCex3 | ATTTTGTTTGTTTAAGTA |
| | (SEQ ID NO: 73) | (SEQ ID NO: 1202) |
| 719 | CMYCex3 | AATAGTTACGGAATTTTT |
| | (SEQ ID NO: 73) | (SEQ ID NO: 1203) |
| 720 | CMYCex3 | AATAGTTATGGAATTTTT |
| | (SEQ ID NO: 73) | (SEQ ID NO: 1204) |
| 721 | CMYCex3 | TTTTTGTGCGTAAGGAAA |
| | (SEQ ID NO: 73) | (SEQ ID NO: 1205) |
| 722 | CMYCex3 | TTTTTGTGTGTAAGGAAA |
| | (SEQ ID NO: 73) | (SEQ ID NO: 1206) |
| 723 | CMYCex3 | AAGGAAAACGATTTTTTT |
| | (SEQ ID NO: 73) | (SEQ ID NO: 1207) |
| 724 | CMYCex3 | AAGGAAAATGATTTTTTT |
| | (SEQ ID NO: 73) | (SEQ ID NO: 1208) |
| 725 | CMYCex3 | TGAGTAATCGTTTATGAA |
| | (SEQ ID NO: 73) | (SEQ ID NO: 1209) |
| 726 | CMYCex3 | TGAGTAATTGTTTATGAA |
| | (SEQ ID NO: 73) | (SEQ ID NO: 1210) |

TABLE 4

Hybridisation olinonucleotides used in the
differentiation between healthy and leukemia
cells (FIG. 3)

| | | |
|---|---|---|
| No: | Gene | Oligo: |

| | | |
|---|---|---|
| 1 | Humos | GTTACCACCGAACTCCAT |
| | (SEQ ID NO: 71) | (SEQ ID NO: 1245) |
| 2 | Humos | GTTACCACCAAACTCCAT |
| | (SEQ ID NO: 71) | (SEQ ID NO: 1246) |
| 3 | Humos | GTTACCACCGAACTCCAT |
| | (SEQ ID NO: 71) | (SEQ ID NO: 1245) |
| 4 | Humos | GTTACCACCAAACTCCAT |
| | (SEQ ID NO: 71) | (SEQ ID NO: 1246) |
| 5 | CDK 4 | TTTAAGGGCGGGAAGTGG |
| | (SEQ ID NO: 70) | (SEQ ID NO: 1247) |
| 6 | CDK 4 | TTTAAGGGTGGGAAGTGG |
| | (SEQ ID NO: 70) | (SEQ ID NO: 1248) |
| 7 | CDK 4 | CCACTTCCCGCCCTTAAA |
| | (SEQ ID NO: 70) | (SEQ ID NO: 1249) |
| 8 | CDK 4 | CCACTTCCCACCCTTAAA |
| | (SEQ ID NO: 70) | (SEQ ID NO: 1250) |
| 9 | CDK 4 | TTTAAGGGCGGGAAGTGG |
| | (SEQ ID NO: 70) | (SEQ ID NO: 1247) |
| 10 | CDK 4 | TTTAAGGGTGGGAAGTGG |
| | (SEQ ID NO: 70) | (SEQ ID NO: 1248) |
| 11 | CDK 4 | CCACTTCCCGCCCTTAAA |
| | (SEQ ID NO: 70) | (SEQ ID NO: 1249) |
| 12 | CDK 4 | CCACTTCCCACCCTTAAA |
| | (SEQ ID NO: 70) | (SEQ ID NO: 1250) |
| 13 | Humos | AAACAAACCGTTCACAAC |
| | (SEQ ID NO: 71) | (SEQ ID NO: 1251) |
| 14 | Humos | AAACAAACCATTCACAAC |
| | (SEQ ID NO: 71) | (SEQ ID NO: 1252) |
| 15 | Humos | AAACAAACCGTTCACAAC |
| | (SEQ ID NO: 71) | (SEQ ID NO: 1251) |
| 16 | Humos | AAACAAACCATTCACAAC |
| | (SEQ ID NO: 71) | (SEQ ID NO: 1252) |
| 17 | Humos | CTCCCCTACGTCCCCCTC |
| | (SEQ ID NO: 71) | (SEQ ID NO: 1253) |
| 18 | Humos | CTCCCCTACATCCCCCTC |
| | (SEQ ID NO: 71) | (SEQ ID NO: 1254) |
| 19 | Humos | CTCCCCTACGTCCCCCTC |
| | (SEQ ID NO: 71) | (SEQ ID NO: 1253) |
| 20 | Humos | CTCCCCTACATCCCCCTC |
| | (SEQ ID NO: 71) | (SEQ ID NO: 1254) |
| 21 | AR | GTAGTATTCGAAGGTAGT |
| | (SEQ ID NO: 69) | (SEQ ID NO: 1255) |
| 22 | AR | GTAGTATTTGAAGGTAGT |
| | (SEQ ID NO: 69) | (SEQ ID NO: 1256) |
| 23 | AR | ACTACCTTCGAATACTAC |
| | (SEQ ID NO: 69) | (SEQ ID NO: 1257) |
| 24 | AR | ACTACCTTCAAATACTAC |
| | (SEQ ID NO: 69) | (SEQ ID NO: 1258) |
| 25 | AR | GTAGTATTCGAAGGTAGT |
| | (SEQ ID NO: 69) | (SEQ ID NO: 1255) |
| 26 | AR | GTAGTTATTTGAAGGTAGT |
| | (SEQ ID NO: 69) | (SEQ ID NO: 1256) |
| 27 | AR | ACTACCTTCGAATACTAC |
| | (SEQ ID NO: 69) | (SEQ ID NO: 1257) |
| 28 | AR | ACTACCTTCAAATACTAC |
| | (SEQ ID NO: 69) | (SEQ ID NO: 1258) |

TABLE 3

Hybridisation olinonucleotides used in differen-
tiation between acute lymphocytic leukemia and
acute myelogenous leukemia (FIGS. 5 and 6).

| | | |
|---|---|---|
| No: | Gene | Oligo: |

| | | |
|---|---|---|
| 1 | N33 | TTGGTTCGGGAAAGGTAA |
| | (SEQ ID NO: 46) | (SEQ ID NO: 1211) |
| 2 | N33 | TTGGTTTGGGAAAGGTAA |
| | (SEQ ID NO: 46) | (SEQ ID NO: 1212) |
| 3 | EGR4 | GGTGGGAAGCGTATTTAT |
| | (SEQ ID NO: 26) | (SEQ ID NO: 1213) |
| 4 | EGR4 | GGTGGGAAGTGTATTTAT |
| | (SEQ ID NO: 26) | (SEQ ID NO: 1214) |
| 5 | N33 | ATTTAGTTCGGGGGAGGA |
| | (SEQ ID NO: 46) | (SEQ ID NO: 1215) |
| 6 | N33 | ATTTAGTTTGGGGGAGGA |
| | (SEQ ID NO: 46) | (SEQ ID NO: 1216) |
| 7 | N33 | TGTTATTTCGGAGGGTTT |
| | (SEQ ID NO: 46) | (SEQ ID NO: 1217) |
| 8 | N33 | TGTTATTTTGGAGGGTTT |
| | (SEQ ID NO: 46) | (SEQ ID NO: 1218) |
| 9 | N33 | GTTTAGTTAGCGGGTTTT |
| | (SEQ ID NO: 46) | (SEQ ID NO: 1219) |
| 10 | N33 | GTTTAGTTAGTGGGTTTT |
| | (SEQ ID NO: 46) | (SEQ ID NO: 1220) |
| 11 | Humos | GAGTTTAACGTAGTAAGG |
| | (SEQ ID NO: 71) | (SEQ ID NO: 1221) |
| 12 | Humos | GAGTTTAATGTAGTAAGG |
| | (SEQ ID NO: 71) | (SEQ ID NO: 1222) |
| 13 | SDC4 | TTTTTATATCGGGTGTGTT |
| | (SEQ ID NO: 54) | (SEQ ID NO: 1223) |
| 14 | SDC4 | TTTTATATTGGGTGTGTT |
| | (SEQ ID NO: 54) | (SEQ ID NO: 1224) |
| 15 | EGR4 | TTATAGTTCGAGTTTTTT |
| | (SEQ ID NO: 26) | (SEQ ID NO: 1225) |
| 16 | EGR4 | TTATAGTTTGAGTTTTTT |
| | (SEQ ID NO: 26) | (SEQ ID NO: 1226) |
| 17 | MPL | TGTAGTGAGTCGAGATTA |
| | (SEQ ID NO: 42) | (SEQ ID NO: 1227) |
| 18 | MPL | TGTAGTGAGTTGAGATTA |
| | (SEQ ID NO: 42) | (SEQ ID NO: 1228) |
| 19 | TP73 | TTTGGTGCGCGTAGAGAA |
| | (SEQ ID NO: 58) | (SEQ ID NO: 1229) |
| 20 | TP73 | TTTGGTGTGTGTAGAGAA |
| | (SEQ ID NO: 58) | (SEQ ID NO: 1230) |
| 21 | CDK 4 | GGGTTTTACGTGGTTGGA |
| | (SEQ ID NO: 70) | (SEQ ID NO: 1231) |
| 22 | CDK 4 | GGGTTTTATGTGGTTGGA |
| | (SEQ ID NO: 70) | (SEQ ID NO: 1232) |
| 23 | CDK 4 | TCCAACCACGTAAAACCC |
| | (SEQ ID NO: 70) | (SEQ ID NO: 1233) |
| 24 | CDK 4 | TCCAACCACATAAAACCC |
| | (SEQ ID NO: 70) | (SEQ ID NO: 1234) |
| 25 | CDK 4 | GGGTTTTACGTGGTTGGA |
| | (SEQ ID NO: 70) | (SEQ ID NO: 1231) |
| 26 | CDK 4 | GGGTTTTATGTGGTTGGA |
| | (SEQ ID NO: 70) | (SEQ ID NO: 1232) |
| 27 | CDK 4 | TCCAACCACGTAAAACCC |
| | (SEQ ID NO: 70) | (SEQ ID NO: 1233) |
| 28 | CDK 4 | TCCAACCACATAAAACCC |
| | (SEQ ID NO: 70) | (SEQ ID NO: 1234) |
| 29 | CSNK2B | AGGAGTTTCGGAGGAAAT |
| | (SEQ ID NO: 67) | (SEQ ID NO: 1235) |
| 30 | CSNK2B | AGGAGTTTTGGAGGAAAT |
| | (SEQ ID NO: 67) | (SEQ ID NO: 1236) |
| 31 | CSNK2B | ATTTCCTCCGAAACTCCT |
| | (SEQ ID NO: 67) | (SEQ ID NO: 1237) |
| 32 | CSNK2B | ATTTCCTCCAAAACTCCT |
| | (SEQ ID NO: 67) | (SEQ ID NO: 1238) |
| 33 | CSNK2B | AGGAGTTTCGGAGGAAAT |
| | (SEQ ID NO: 67) | (SEQ ID NO: 1235) |
| 34 | CSNK2B | AGGAGTTTTGGAGGAAAT |
| | (SEQ ID NO: 67) | (SEQ ID NO: 1236) |
| 35 | CSNK2B | ATTTCCTCCGAAACTCCT |
| | (SEQ ID NO: 67) | (SEQ ID NO: 1237) |
| 36 | CSNK2B | ATTTCCTCCAAAACTCCT |
| | (SEQ ID NO: 67) | (SEQ ID NO: 1238) |
| 37 | Tubulin | TACTTCAACGTCCTAATA |
| | (SEQ ID NO: 64) | (SEQ ID NO: 1239) |
| 38 | Tubulin | TACTTCAACATCCTAATA |
| | (SEQ ID NO: 64) | (SEQ ID NO: 1240) |
| 39 | Tubulin | TACTTCAACGTCCTAATA |
| | (SEQ ID NO: 64) | (SEQ ID NO: 1239) |
| 40 | Tubulin | TACTTCAACATCCTAATA |
| | (SEQ ID NO: 64) | (SEQ ID NO: 1240) |
| 41 | ELK1 | TTTATTTTCGTTTTTGGG |
| | (SEQ ID NO: 63) | (SEQ ID NO: 1241) |
| 42 | ELK1 | TTTATTTTTGTTTTTGGG |
| | (SEQ ID NO: 63) | (SEQ ID NO: 1242) |
| 43 | ELK1 | TTTATTTTCGTTTTTGGG |
| | (SEQ ID NO: 63) | (SEQ ID NO: 1241) |
| 44 | ELK1 | TTTATTTTTGTTTTTGGG |
| | (SEQ ID NO: 63) | (SEQ ID NO: 1242) |
| 45 | Tubulin | CAATACTCCGTCCATTAA |
| | (SEQ ID NO: 64) | (SEQ ID NO: 1243) |
| 46 | Tubulin | CAATACTCCATCCATTAA |
| | (SEQ ID NO: 64) | (SEQ ID NO: 1244) |
| 47 | Tubulin | CAATACTCCGTCCATTAA |
| | (SEQ ID NO: 64) | (SEQ ID NO: 1243) |
| 48 | Tubulin | CAATACTCCATCCATTAA |
| | (SEQ ID NO: 64) | (SEQ ID NO: 1244) |

### DESCRIPTION OF THE FIGURES

FIG. 1

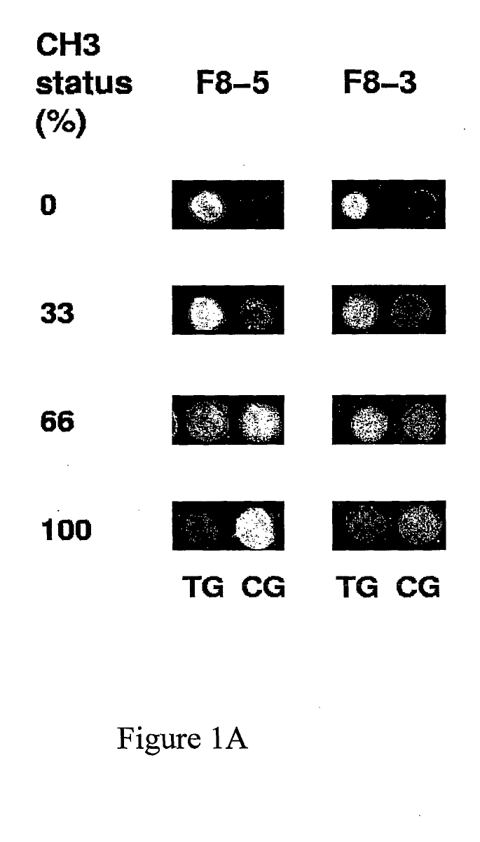

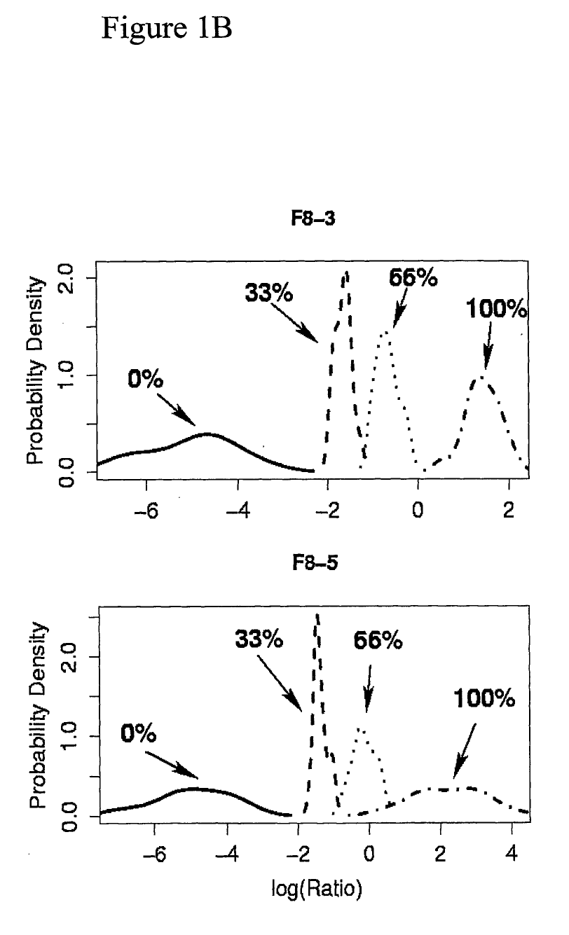

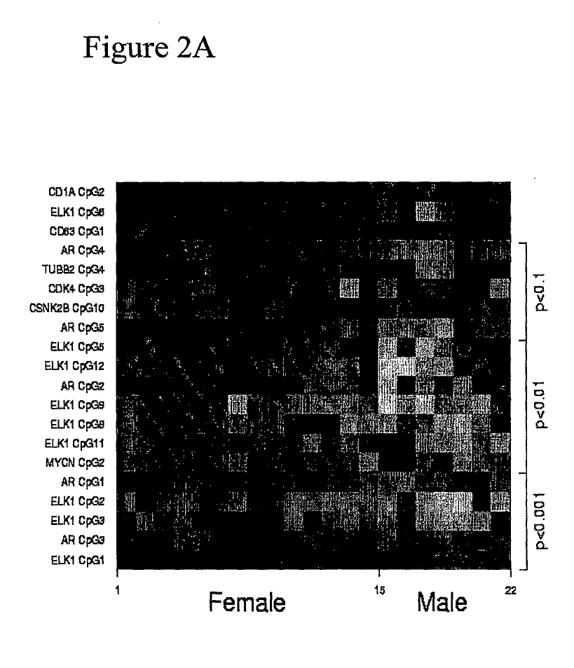

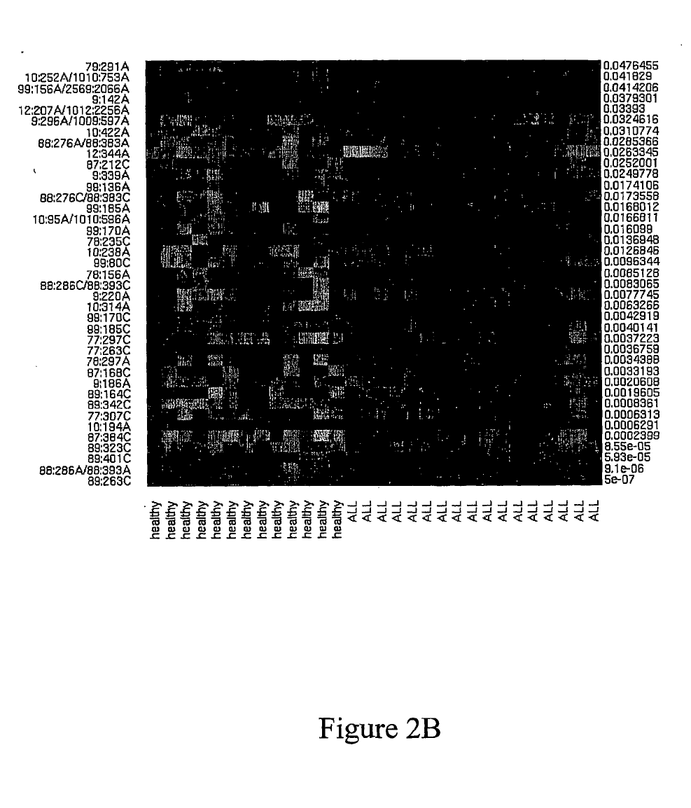

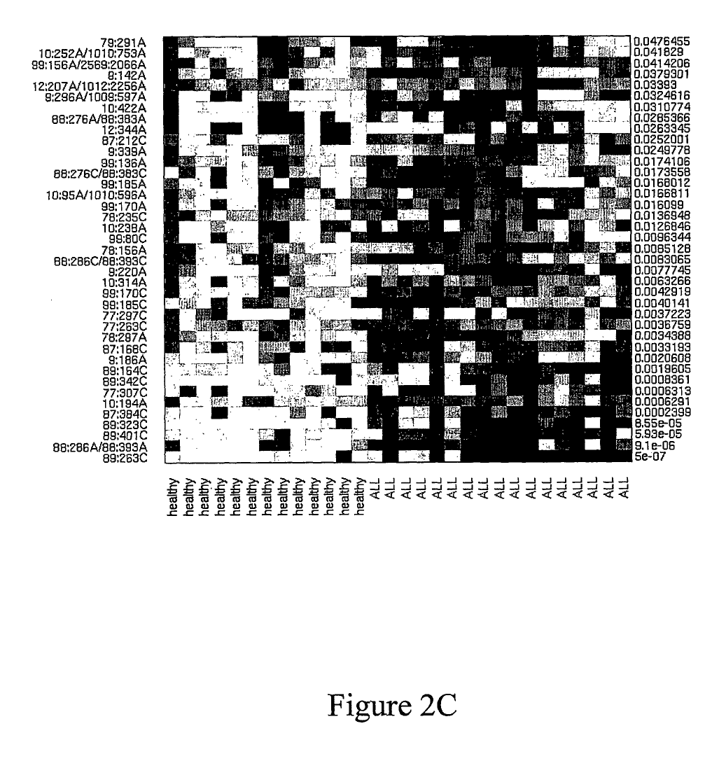

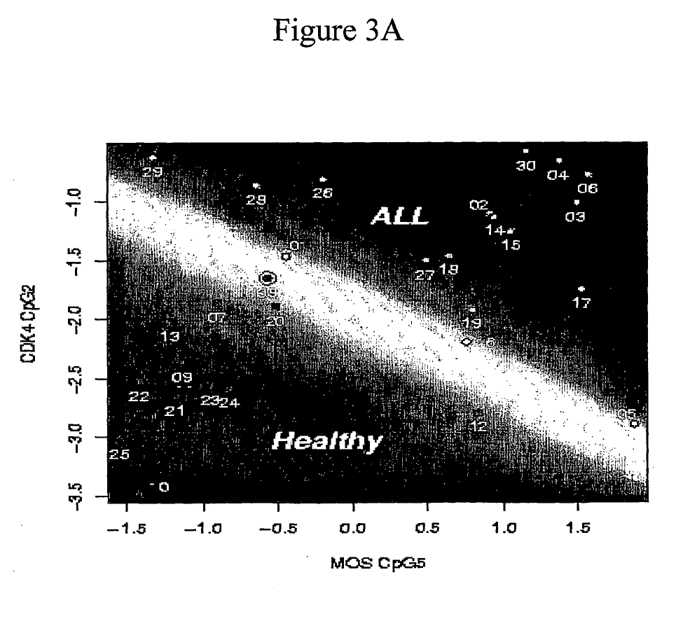

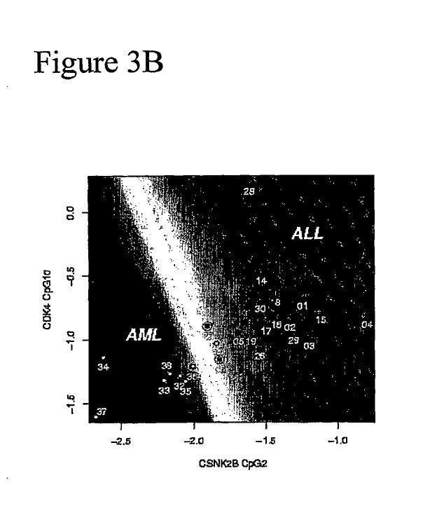

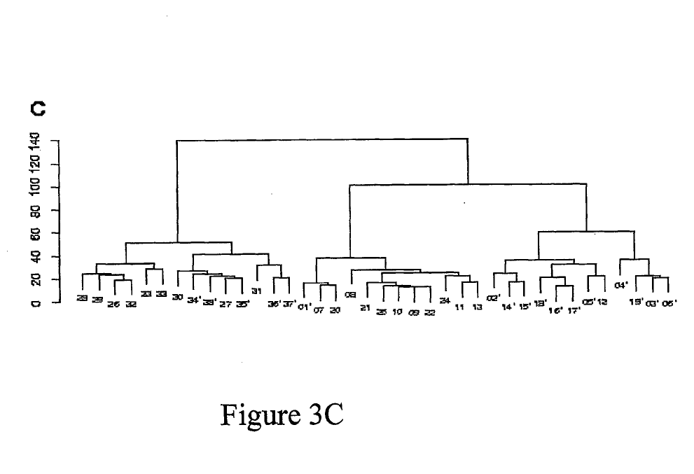

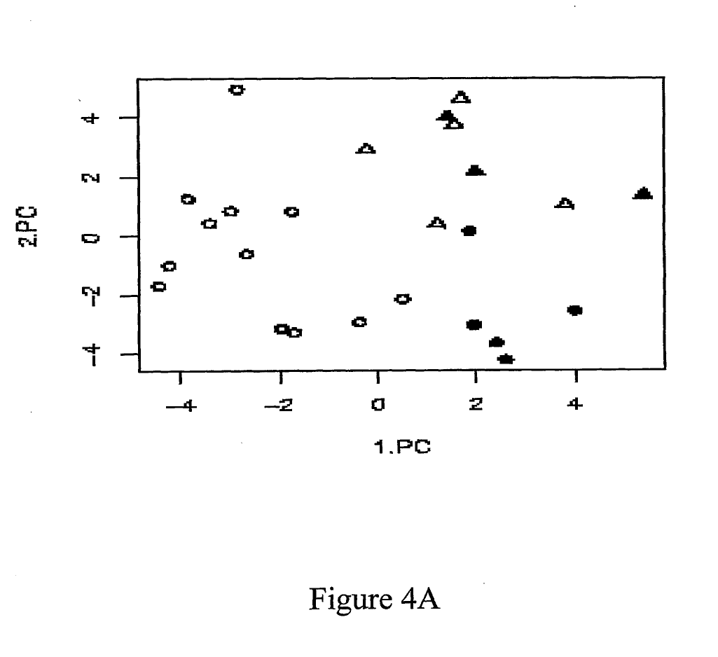

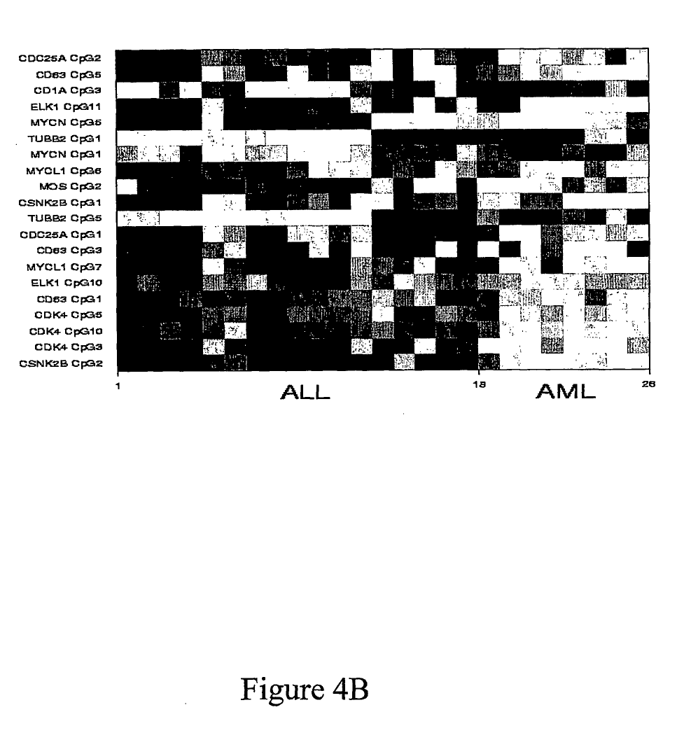

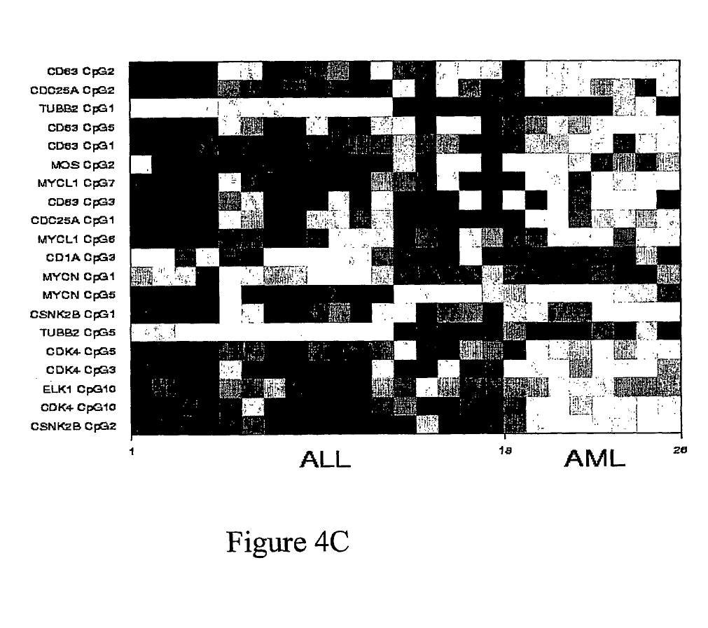

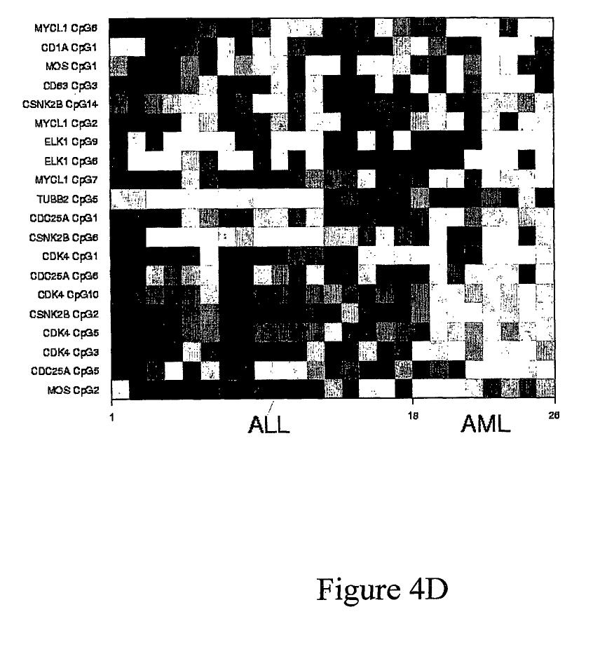

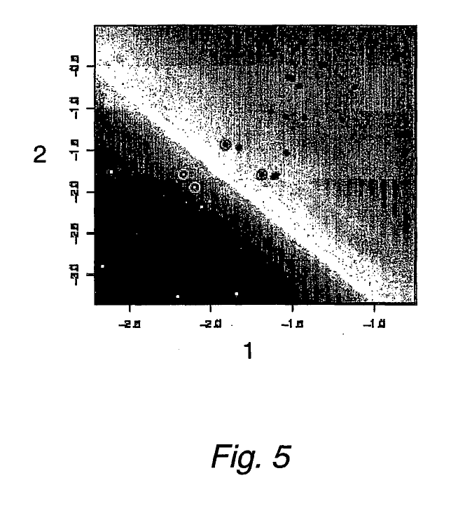

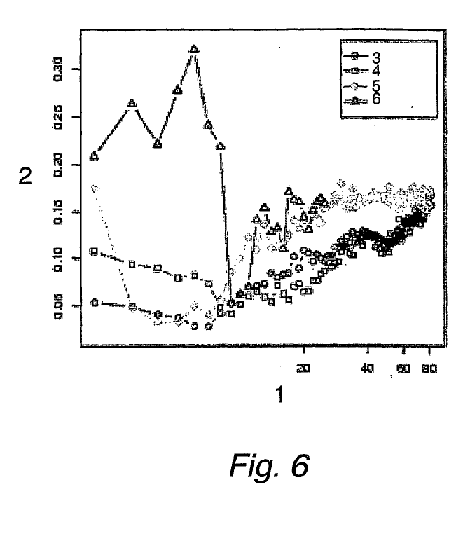

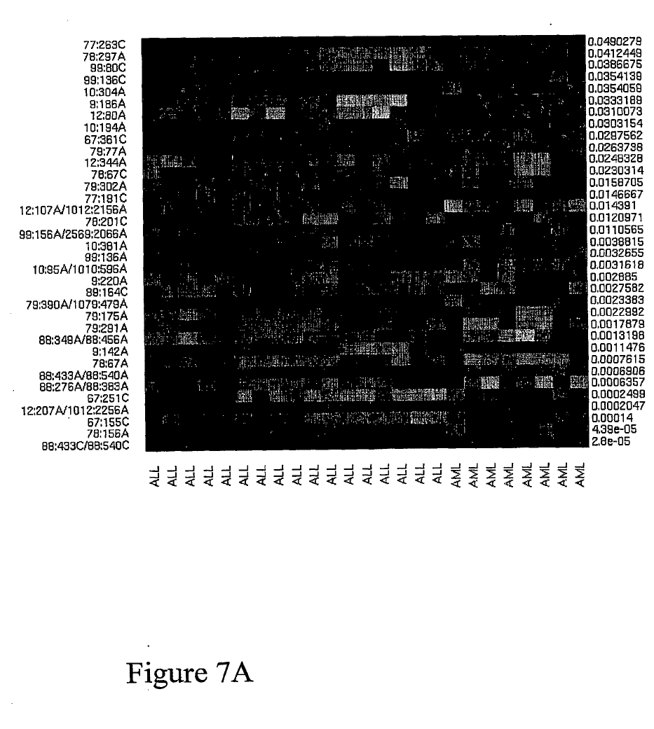

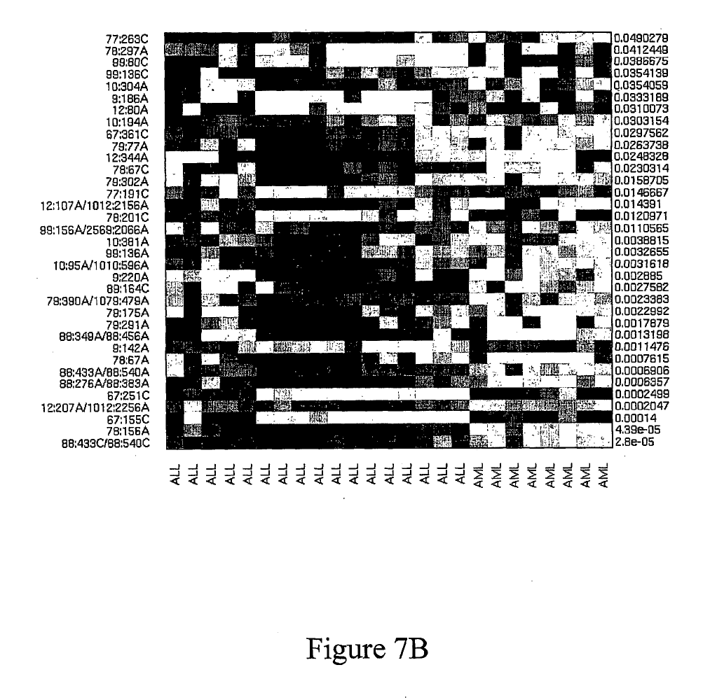

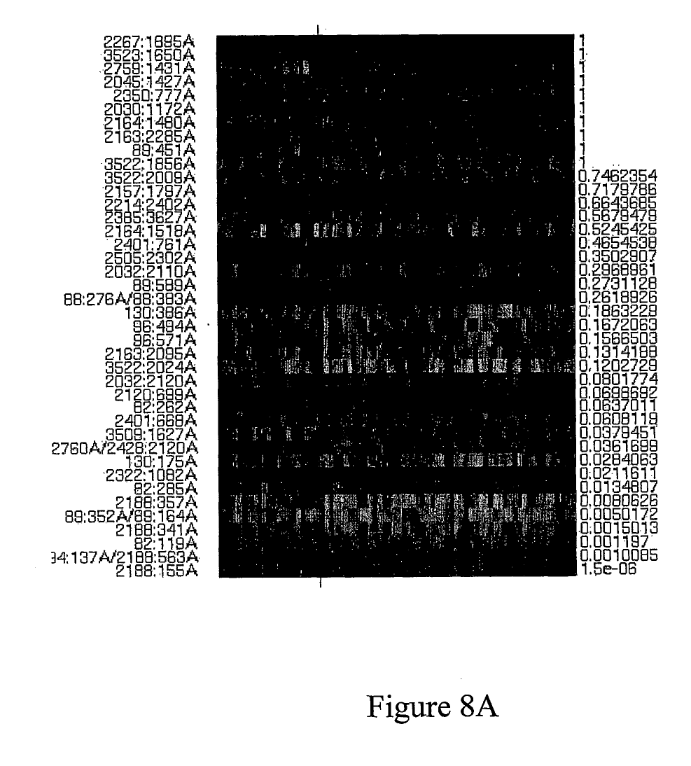

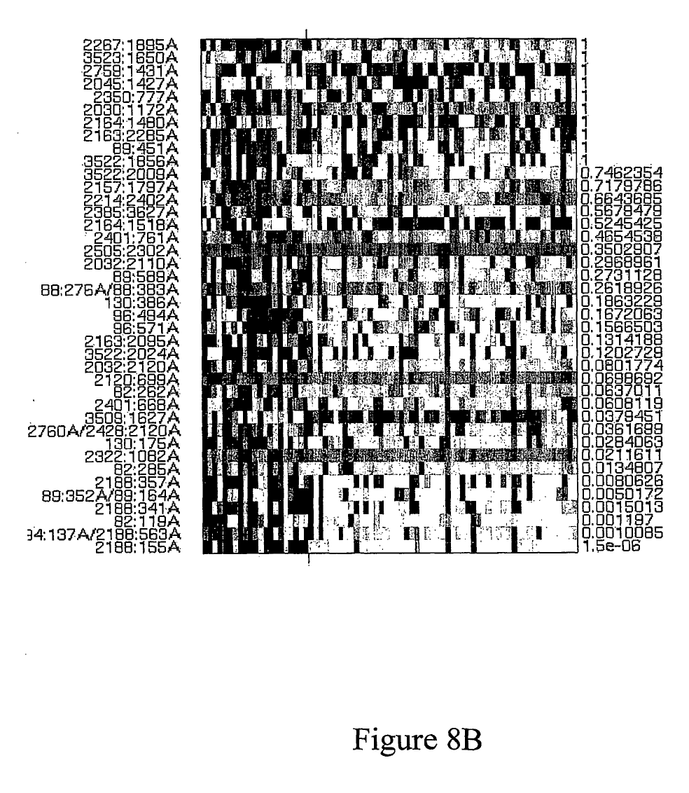

Methylation analysis and quantification of two CpG dinucleotides in exon 14 of the human factor VIII gene. For calibration purpose a series of hybridisations was performed with mixtures of artificially up- and down-methylated DNA fragments of the factor VIII exon 14 gene. Down- and up-methylated DNA fragments were mixed at ratios: 0:3, 1:2, 2:1, 3:0, representing a methylation status of 100%, 66%, 33% and 0%, respectively. A, Methylation detection by oligonucleotide microarray hybridisation. The fluorescence signals of the CG and TG version of the factor VIII exon 14 oligonucleotides F8-5 (TTATTAACGGGAAATAAT, TTATTAATGGGAAATAAT) and F8-3 (AATAAGTTCGAAATAGAA, AATAAGTTTGAAATAGAA) are shown which are generated by samples reflecting the methylation status 0%, 33%, 66% and 100%. The hybridisation signals are shown in a false colour image with the colours blue, green and yellow indicating fluorescence signal ranges at 635 nm of 200 to 800, 800 to 2000 and 2000 to 8000, respectively. B, Distribution and probability of measurement of different methylated DNA for two CpG methylation. For each CpG position two kinds of detection oligomers were used. Oligomers that hybridise if the CpG was methylated are referred to as CG oligomer and the oligomers that hybridise if the CpG was not methylated are referred to as TG oligos. For the 4 kinds of compounds 59, 36, 40, 63 identical slides were made. The log-ratio of the CG and the TG detection oligomer hybridisation intensity was calculated and then averaged for experimental subgroups each containing 3 identical experiments. The distribution function of the CG:TG ratios shows that measurement values of the different mixtures are well separated and therefore allow a high resolution detection of the methylation level of a single CpG. This is an essential prerequisite for methylation dependent class prediction or class discovery. Taking into account only the 100% and 0% methylated DNA, and averaging for the 22 CpG sites investigated in the calibration experiments, the average error for methylation detection is 4%. The log-ratios are not grouped symmetrically around zero but shifted towards negative values. We assume that the energetically different effects of G-T and A-C mismatches allow hybridisations of the methylated allele to the oligonucleotide representing the unmethylated more easily than vice versa.

FIG. 2

Gender separation and CpG site prediction of ALL/AML and healthy blood samples. High probability of methylation corresponds to red, uncertainty to black and low probability to green. The labels on the left side of the plot are gene and CpG identifiers. The labels on the right side give the significance of the difference between the means of the two groups. Each row corresponds to a single CpG and each column to the methylation levels of one sample.

FIG. 2A, Gender separation. The 20 CpG sites with the most significant difference between female and male samples are shown. Only non cell lines were used. As expected the significant CpG dinucleotides come from the two X-chromosome genes (ELK1, AR).

FIG. 2B, differentiation between healthy samples and ALL samples. The 54 CpG sites with the most significant difference between healthy and ALL samples are shown. High probability of methylation corresponds to red, uncertainty to black and low probability to green. The labels on the left side of the plot are gene and CpG identifiers. The labels on the right side give the significance of the difference between the means of the two groups. Each row corresponds to a single CpG and each column to the methylation levels of one sample.

FIG. 2C shows FIG. 2B reproduced in greyscale. High probability of methylation corresponds to black, uncertainty to grey and low probability to white. The labels on the left side of the plot are gene and CpG identifiers. The labels on the right side give the significance of the difference between the means of the two groups. Each row corresponds to a single CpG and each column to the methylation levels of one sample.

Legend FIG. 2A:

1—Female

2—Male

Legend FIG. 2B:

1—Healthy

2—ALL

FIG. 3

Class prediction and class discovery of blood samples. A, The plot shows a SVM trained on the two most significant CpG sites for the healthy-ALL discrimination using all available healthy and ALL samples as training data. The red points are healthy, the yellow ones ALL samples. Circled points are the support vectors defining the white borderline between the green area of healthy prediction and the blue area of an ALL prediction. The colour intensity corresponds to the prediction strength. B, Support Vector Machine for AML-ALL class prediction. The red points are ALL, the yellow ones AML samples. Circled points are the support vectors defining the white borderline between the green area of ALL prediction and the blue area of an AML prediction. The colour intensity corresponds to the prediction strength. C, Class discovery. The figure shows a hierarchical clustering of all available samples. Healthy individuals are coloured green, patients with ALL red and patients with AML blue. Asterisks indicate cell line samples. The feature space consisted of all CpG sites except those from the two X-chromosomal genes. The diagnosis was unknown to the algorithm.

Legend FIG. 3A: t-Test ranking (Healty vs. ALL)

1—MOS CpG5

2—CDK4 CpG2

Legend FIG. 3B: t-Test ranking (AML vs. ALL)

1—CSNK2B CpG2

2—CDK4 CpG10

FIG. 4: Feature selection methods.

FIG. 4A shows principle component analysis. The entire data set was projected onto its first 2 principle components. Circles represent cell lines, triangles primary patient tissue. Filled circles or triangles are AML, empty ones ALL samples.

FIG. 4B: Fisher criterion. The 20 highest ranking CpG sites according to the Fisher criterion are shown. The highest ranking features are on the bottom of the plot. High probability of methylation corresponds to black, uncertainty to grey and low probability to white.

FIG. 4C: Two sample t-test.

FIG. 4D: Backward elimination.

FIG. 5

Support Vector Machine on two best features of the Fisher criterion. The plot shows a SVM trained on the two highest ranking CpG sites according to the Fisher criterion with all ALL and AML samples used as training data. The black points are AML, the grey ones ALL samples. Circled points are the support vectors defining the white borderline between

the areas of AML and ALL prediction. The grey value of the background corresponds to the prediction strength.

Legend FIG. 5:

1—CSNK2B CpG2

2—CDK4 CpG3

FIG. 6

Dimension dependence of feature selection performance. The plot shows the generalisation performance of a linear SVM with four different feature selection methods against the number of selected features. The x-axis is scaled logarithmically and gives the number of input features for the SVM, starting with two. The y-axis gives the achieved generalisation performance. Note that the maximum number of principle components corresponds to the number of available samples.

Legend FIG. 6:

1—Feature Number

2—Test Error

3—Fisher Criterion

4—t-Test

5—Backward Elimination

6—Principle Component Analysis

FIG. 7A

Differentiation between healthy and acute lymphocytic leukemia samples. High probability of methylation corresponds to red, uncertainty to black and low probability to green. The labels on the left side of the plot are gene and CpG identifiers. The labels on the right side give the significance of the difference between the means of the two groups. Each row corresponds to a single CpG and each column to the methylation levels of one sample.

FIG. 7B

Differentiation between acute lymphocytic leukemia and acute myelogenous leukemia, this shows FIG. 7A reproduced in greyscale. High probability of methylation corresponds to black, uncertainty to grey and low probability to white. The labels on the left side of the plot are gene and CpG identifiers. The labels on the right side give the significance of the difference between the means of the two groups. Each row corresponds to a single CpG and each column to the methylation levels of one sample.

FIG. 8A

Differentiation between acute lymphocytic leukemia and acute myelogenous leukemia samples. High probability of methylation corresponds to red, uncertainty to black and low probability to green. The labels on the left side of the plot are gene and CpG identifiers. The labels on the right side give the significance of the difference between the means of the two groups. Each row corresponds to a single CpG and each column to the methylation levels of one sample.

FIG. 8B

Differentiation between acute lymphocytic leukemia and acute myelogenous leukemia, this shows FIG. 8A reproduced in greyscale. High probability of methylation corresponds to red, uncertainty to black and low probability to green. The labels on the left side of the plot are gene and CpG identifiers. The labels on the right side give the significance of the difference between the means of the two groups. Each row corresponds to a single CpG and each column to the methylation levels of one sample.

1. A method for detecting and differentiating between hematopoietic cell proliferative disorders associated with at least one gene and/or their regulatory regions from the group comprising ABL1, ABL1, APAF1, APC, AR, ARHI, BAK1, BAX, BCL2, CASP10, CASP8, CASP9, CCND2, CDC2, CDC25A, CDH1, CDH3, CDK4, CDKN1A, CDKN1B (p27Kip1), CDKN1C, CDKN2a, CDKN2B, CSNK2B, DAPK1, EGR4, ELK1, ESR1, FOS, GPIb beta, GRP37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3 in a subject, said method comprising contacting a target nucleic acid in a biological sample obtained from said subject with at least one reagent or a series of reagents, wherein said reagent or series of reagents, distinguishes between methylated and non methylated CpG dinucleotides within the target nucleic acid.

2. A method according to claim 1 wherein, said method differentiates between normal hematopoietic cells and proliferative disorder hematopoietic cells.

3. A method according to claim 1, wherein said method differentiates between acute lymphocytic leukemia and acute myelogenous leukemia.

4. Use of methods according to claim 1 wherein, CDKN1A, CDK 4, AR are used to differentiate between healthy hematopoietic cells and proliferative disorder hematopietic cells.

5. Use of methods according to claim 1 wherein, N33, EGR4, CDKN1A, SDC4, MPL, TP 73, BAK 1, CSNK2B, ARH1, CASP 10 are used to differentiate between acute lymphocytic leukemia and acute myelogenous leukemia.

6. A method according to claim 1 comprising the following steps:
obtaining a biological sample containing genomic DNA;
extracting the genomic DNA;
converting cytosine bases in the genomic DNA sample which are unmethylated at the 5-position, by treatment, to uracil or another base which is dissimilar to cytosine in terms of base pairing behavior;
fragments of the pretreated genomic DNA are amplified;
identification of the methylation status of one or more cytosine positions.

7. The method according to claim 6, characterized in that the reagent is a solution of bisulfite, hydrogen sulfite or disulfite.

8. The method as recited in claim 6, characterized in that the amplification is carried out by means of the polymerase chain reaction (PCR).

9. The method as recited in one of the claim 6, characterized in that the amplification is carried out by means of a heat-resistant DNA polymerase.

10. The method as recited in claim 6, characterized in that more than ten different fragments having a length of 100-2000 base pairs are amplified.

11. The method as recited in claim 6, wherein the amplification step is carried out using a set of primer oligonucleotides comprising SEQ ID NO:387 through SEQ ID NO:534.

12. The method as recited claim 6, characterized in that the amplification of several DNA segments is carried out in one reaction vessel.

13. The method as recited in claim 6, characterized in that the amplification step preferentially amplifies DNA which is of particular interest in healthy and/or diseased hematopoietic cells, based on the specific genomic methylation status of hematopoietic cells, as opposed to background DNA.

14. The method according to claim 6, characterized in that the methylation status within at least one gene and/or their regulatory regions from the group comprising ABL1, ABL1, APAF1, APC, AR, ARHI, BAK1, BAX, BCL2, CASP10, CASP8, CASP9, CCND2, CDC2, CDC25A, CDH1, CDH3, CDK4, CDKN1A, CDKN1B (p27Kip1), CDKN1C, CDKN2a, CDKN2B, CSNK2B, DAPK1, EGR4, ELK1, ESR1, FOS, GPIb beta, GRP37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3 is detected by hybridization of each amplificate to an oligonucleotide or peptide nucleic acid (PNA)-oligomer.

15. A method according to claim 14, characterized in that the oligonucleotide or peptide nucleic acid (PNA)-oligomer is taken from the group comprising SEQ ID NO:535 to SEQ ID NO:1258.

16. The method according to claim 6, characterized in that the amplificates are labelled.

17. The method as recited in claim 16, characterized in that the labels of the amplificates are fluorescence labels.

18. The method as recited in claim 16, characterized in that the labels of the amplificates are radionuclides.

19. The method as recited in claim 16, characterized in that the labels of the amplificates are detachable molecule fragments having a typical mass which are detected in a mass spectrometer.

20. The method as recited claim 6, characterized in that the amplificates or fragments of the amplificates are detected in the mass spectrometer.

21. The method as recited in claim 19, characterized in that the produced fragments have a single positive or negative net charge.

22. The method as recited in claim 19, characterized in that detection is carried out and visualized by means of matrix assisted laser desorption/ionization mass spectrometry (MALDI) or using electron spray mass spectrometry (ESI).

23. A method according to claim 1 comprising the following steps;
a) obtaining a biological sample containing genomic DNA;
b) extracting the genomic DNA;
c) digesting the genomic DNA comprising at least one or more CpGs of the genes ABL1, ABL1, APAF1, APC, AR, ARHI, BAK1, BAX, BCL2, CASP10, CASP8, CASP9, CCND2, CDC2, CDC25A, CDH1, CDH3, CDK4, CDKN1A, CDKN1B (p27Kip1), CDKN1C, CDKN2a, CDKN2B, CSNK2B, DAPK1, EGR4, ELK1, ESR1, FOS, GPIb beta, GRP37, GSK3β, GSTP1, HIC-1, HOXA5, IGF2, MDR1, MGMT, MLH1, MOS, Humos, MPL, MYC, MYCL1, MYOD1, N33, PITX2, PML, PMS2, PRAME, PTEN, RB1, RBL2, SDC4, SFN, TCL1A, TGFBR2, TP73, WT1, N-MYC, L-MYC, C-ABL, ELK1, Tubulin, CSF1, CD1R3, CSNK2B, Me491/TD63, AR, CDK 4, Humos, CDC25A, CMYCex3 with one or more methylation sensitive restriction enzymes;
d) detection of the DNA fragments generated in the digest of step c).

24. A method according to claim 23, wherein the DNA digest is amplified prior to Step d.

25. The method as recited in claim 24, characterized in that the amplification is carried out by means of the polymerase chain reaction (PCR).

26. The method as recited in one of the claims claim 24 and/or 25, characterized in that the amplification of more than one DNA fragments is carried out in one reaction vessel.

27. The method as recited in one of the claims claim 24 though 26, characterized in that the polymerase is a heat-resistant DNA polymerase.

28. An isolated nucleic acid of a pretreated genomic DNA according to one of the sequences taken from the group comprising SEQ ID NO:95 to SEQ ID NO:386 and sequences complementary thereto.

29. An oligomer, in particular an oligonucleotide or peptide nucleic acid (PNA)-oligomer, said oligomer comprising at least one base sequence of at least 10 nucleotides which hybridizes to or is identical to a pretreated genomic DNA acccording to one of the SEQ ID NO:95 to SEQ ID NO:386 according to claim 28.

30. The oligonucleotide as recited in claim 29; wherein the base sequence includes at least one CpG or TpG dinucleotide sequence.

31. The oligonucleotide as recited in claim 30; characterized in that the cytosine of the at least one CpG or TpG dinucleotide is/are located approximately in the middle third of the oligomer.

32. An oligomer, in particular an oligonucleotide or peptide nucleic acid (PNA)-oligomer, according to one of the sequences taken from the group comprising SEQ ID NO:535 to SEQ ID NO: 1258.

33. A set of oligonucleotides, comprising at least two oligonucleotides according to claim 29.

34. A set of oligonucleotides, comprising at least two oligonucleotides according to SEQ ID NO:1245, 1245, 1246, 1246, 1247, 1247, 1248, 1248, 1249, 1249, 1250, 1250, 1251, 1251, 1252, 1252, 1253, 1253, 1254, 1254, 1255, 1255, 1256, 1256, 1257, 1257, 1258, 1258.

35. One or more isolated nucleic acid(s) taken from the group according to SEQ ID NO:88-94.

36. A set of oligonucleotides, comprising at least two oligonucleotides according to SEQ ID NO:1211-1231, 1231, 1232, 1232, 1233, 1233, 1234, 1234, 1235, 1235, 1236, 1236, 1237, 1237, 1238, 1238, 1239, 1239, 1240, 1240, 1241, 1241, 1242, 1242, 1243, 1243, 1244, 1244.

37. One or more isolated nucleic acid(s) taken from the group according to SEQ ID NO:74-87.

38. A set of oligomers, peptide nucleic acid (PNA)-oligomers and/or isolated nucleic acids as recited in claims 33 through 35 and 37, comprising oligomers for detecting the methylation state of all CpG dinucleotides within one or more of the sequences according to SEQ ID NO:1 to SEQ ID NO:73 and sequences complementary thereto.

39. Use of a set of oligomers or peptide nucleic acid (PNA)-oligomers according to any of claims 29 through 34 and 36 as probes for determining the cytosine methylation state and/or single nucleotide polymorphisms (SNPs) of sequences according to SEQ ID NO:1 to SEQ ID NO:73 and sequences complementary thereto.

40. Use of a set of oligonucleotides according to claim 34 or nucleic acid(s) according to claim 35 for the differentiation between healthy hematopoietic cells and proliferative disorder hematopoietic cells.

41. Use of a set of oligonucleotides according to claim 36 or nucleic acid(s) according to claim 37 for the differentiation between acute lymphocytic leukemia and acute myelogenous leukemia.

42. A set of at least two oligonucleotides or peptide nucleic acid (PNA)-oligomers as recited in claim 29, as primer oligonucleotides for the amplification of DNA sequences of one of SEQ ID NO:95 to SEQ ID NO:386 according to claim 28 and/or sequences complementary thereto and segments thereof.

43. Use of a pretreated genomic DNA according to claim 28 for the determination of the methylation status of a corresponding genomic DNA and/or detection of single nucleotide polymorphisms (SNPs).

44. A set of olignucleotides or peptide nucleic acid (PNA)-oligomers as recited in claims 33, 34 or 36 characterized in that at least one oligonucleotide is bound to a solid phase.

45. A set of oligonucleotides or peptide nucleic acid (PNA)-oligomers as recited in claims 33, 34 or 36, characterized in that all members of the set are bound to a solid phase.

46. A method for manufacturing an arrangement of different oligomers or peptide nucleic acid (PNA)-oligomers (array) for analyzing diseases associated with the corresponding genomic methylation status of the CpG dinucleotides within one of the SEQ ID NO:1 to SEQ ID NO:73 and sequences complementary thereto, wherein at least one oligomer according to any of the claims 33, 34 or 36 is coupled to a solid phase.

47. An arrangement of different oligomers or peptide nucleic acid (PNA)-oligomers (array) obtainable according to cams claim 44 and 45.

48. An array of different oligonucleotide- and/or PNA-oligomer sequences as recited in claim 47, characterized in that these are arranged on a plane solid phase in the form of a rectangular or hexagonal lattice.

49. A nucleic acid or peptide nucleic acid array for the analysis of hematopoeitic cell proliferative disorders associated with the methylation state of genes comprising at least one nucleic acid according to claim 48.

50. The array as recited in claim 47, characterized in that the solid phase surface is composed of silicon, glass, polystyrene, aluminum, steel, iron, copper, nickel, silver, or gold.

51. A kit comprising a bisulfite (=disulfite, hydrogen sulfite) reagent as well as oligonucleotides and/or PNA-oligomers according to one of the claims 29 through 37.

52. The use of oligonucleotides or peptide nucleic acid (PNA)-oligomers according to SEQ ID NO:74 to SEQ ID NO:94 and SEQ ID NO:535 to SEQ ID NO:1258 for the detection of a predisposition to, differentiation between subclasses, diagnosis, prognosis, treatment and/or monitoring of hematopoietic cell proliferative disorders.

53. A DNA sequence according to one of the sequences taken from the group comprising SEQ ID NO:95 to SEQ ID NO:386 and sequences complementary thereto for use in the analysis of cytosine methylation within said nucleic acid for the detection of a predisposition to, differentiation between subclasses, diagnosis, prognosis, treatment and/or monitoring of hematopoietic cell proliferative disorders.
